SEQUENCE LISTING (27 sequences)

SEQ ID NO: 1 - protein, 10 aa, synthetic construct
GFTVSNSVMA

SEQ ID NO: 2 - protein, 16 aa, synthetic construct
IINSIGITNYADSVKG

SEQ ID NO: 3 - protein, 6 aa, synthetic construct
NFDRIY

SEQ ID NO: 4 - protein, 114 aa, synthetic construct
QVQLVESGGGLVQPGGSLRLSCAASGFTVSNSVMAWYRQTPGNEREFVAIINSIGITNYADSVKGRFTISRDNSKNTLYLQMNSLRAEDTAVYVCNRNFDRIYWGQGTLVTVSS

SEQ ID NO: 5 - protein, 8 aa, synthetic construct
GSIFSINA

SEQ ID NO: 6 - protein, 7 aa, synthetic construct
FTGDTNT

SEQ ID NO: 7 - protein, 14 aa, synthetic construct
AADVQLFSRDYEFY

SEQ ID NO: 8 - protein, 120 aa, synthetic construct
EVQLVESGGGEVQPGGSLRLSCAASGSIFSINAMGWYRQAPGKQRELVAGFTGDTNTIYAESVKGRFTISRDNAKNTVYLQMSSLRAEDTAVYYCAADVQLFSRDYEFYWGQGTLVTVKP

SEQ ID NO: 9 - protein, 117 aa, synthetic construct
QVQLVESGGGLVQPGGSLRLSCEASGTTFSRDVMGWYRQAPGKQRELVAIISRGGSTNYADSVKGRFTISRDNSKNTLYLQMNSLRAEDTAVYYCNANTATWGRVFWGQGTLVTVSS

SEQ ID NO: 10 - protein, 114 aa, synthetic construct; identical to SEQ ID NO: 4

SEQ ID NO: 11 - protein, 216 aa, synthetic construct
APELLGGPSVFLFPPKPKDTLMASRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 216 aa, synthetic construct
APELLGGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 15 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHATQKSLSLSPG

SEQ ID NO: 16 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 17 - protein, 216 aa, synthetic construct
APEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 216 aa, synthetic construct
APEAEGAPSVFLFPPKPKDTLMISRTPEVTCVVVDVSHEDPEVKFNWYVDGVEVHNAKTKPREEQYNSTYRVVSVLTVLAQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 216 aa, synthetic construct
APEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPG

SEQ ID NO: 20 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 8 aa, synthetic construct
GYIFNSCG

SEQ ID NO: 22 - protein, 7 aa, synthetic construct
ISGDGDT

SEQ ID NO: 23 - protein, 9 aa, synthetic construct
AVCYNLETY

SEQ ID NO: 24 - protein, 115 aa, synthetic construct
QVQLQESGGGSVQAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSS

SEQ ID NO: 25 - protein, 15 aa, synthetic construct
GGGGSGGGGSGGGGS

SEQ ID NO: 26 - protein, 12 aa, synthetic construct
GGSGGSGGSGGS

SEQ ID NO: 27 - protein, 9 aa, synthetic construct
GGSGGSGGS

(19) United States
(12) Patent Application Publication
(10) Pub. No.: US 2026/0055180 A1
(43) Pub. Date: Feb. 26, 2026

(54) KIDNEY TARGETING ANTIBODIES

(71) Applicant: ABDERA THERAPEUTICS INC., South San Francisco, CA (US)

(72) Inventors: Michael J. ABRAMS, Custer, WA (US); Emma Jane CUMMINS, Vancouver, BC (CA); Adam Daniel JUDGE, Bainbridge Island, WA (US)

(21) Appl. No.: 19/104,559

(22) PCT Filed: Aug. 21, 2023
(86) PCT No.: PCT/US2023/072583

§ 371 Date: Feb. 18, 2025

Related U.S. Application Data
(60) Provisional application No. 63/373,188, filed on Aug. 22, 2022.

(51) Int. Cl. C07K 16/28; A61K 39/00

(52) U.S. Cl. CPC C07K 16/28; A61K 2039/505; C07K 2317/24; C07K 2317/31; C07K 2317/52; C07K 2317/569; C07K 2317/92

(57) ABSTRACT

Described herein are antibodies and immunoconjugates capable of targeting antigens within the kidney. The antibodies described herein generally a polypeptide, wherein the polypeptide comprises: an antigen-binding domain (e.g., immunoglobulin single-chain domain antibody) and a Fc domain, wherein the antigen-binding domain binds an antigen present within the kidney.

### CROSS_REFERENCE

This application claims the benefit of U.S. Provisional Application Ser. No. 63/373,188 filed on Aug. 22, 2022, which is incorporated by reference herein in its entirety.

### BACKGROUND

Kidney diseases have far-reaching effects, encompassing impaired kidney function, fluid imbalances, and heightened cardiovascular risk. Complications include anemia, bone issues, and metabolic disruptions. The chronic nature of these diseases impacts both physical and psychological well-being while increasing healthcare costs. Addressing their impact necessitates comprehensive approaches to detection, management, and treatment.

Ensuring effective access and delivery of therapeutic antibodies to target kidney sites is a complex task due to the kidney's intricate structure. The glomerular filtration barrier limits the passage of larger molecules like antibodies into the kidney tissue, while the renal tubular system poses additional transport challenges. The general approach to overcoming such barriers generally focuses on exploring specialized delivery systems and/or local routes of administration or delivery.

### SUMMARY

Overcoming access and delivery hurdles presents a challenge for enhancing the efficacy of antibody-based therapies for kidney-related diseases. Provided herein are antibodies capable of targeting antigens (e.g., FOLR1) present within the kidney. Conventional antibodies (e.g., IgG having a molecular weight of 150,000 Daltons) are generally considered to be beyond glomerular filtration or significant kidney localization and are generally restricted to the apical (luminal) surface of the kidney. For example, kidney uptake of IgG antibodies and large proteins is substantively limited.

Described herein is an antibody comprising: an antigen-binding domain and a Fc domain, wherein the antigen-binding domain binds an antigen present within the kidney, wherein the antibody, wherein the antibody possesses a molecular weight of less than 110,000 Daltons. In certain embodiments, the antigen-binding domain comprises an immunoglobulin single-chain variable domain polypeptide. In certain embodiments, the immunoglobulin single-chain variable domain polypeptide comprises a VHH. In certain embodiments, the Fc domain comprises a CH2 domain and CH3 domain. In certain embodiments, the Fc domain comprises a CH3 domain. In certain embodiments, the Fc domain comprises a CH2 domain. In certain embodiments, the Fc domain comprises an alteration to one or more amino acid residues that reduces an effector function of the Fc domain. In certain embodiments, the alteration to one or more amino acid residues that reduces the effector function of the immunoglobulin heavy chain constant region is an alteration that reduces complement dependent cytotoxicity (CDC), antibody-dependent cell-cytotoxicity (ADCC), antibody-dependent cell-phagocytosis ADCP, or a combination thereof. In certain embodiments, the alteration to one or more amino acid residues that reduces the effector function of the immunoglobulin heavy chain constant region is selected from the list consisting of: (a) 297A, 297Q, 297G, or 297D, (b) 279F, 279K, or 279L, (c) 228P, (d) 235A, 235E, 235G, 235Q, 235R, or 2355, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) K322A, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) E233P, (ooo) L234A, L235E, G237A, A330S, and P331S or (ppp) any combination of (a)-(ppp), per EU numbering. In certain embodiments, the alteration to one or more amino acid residues that reduces the effector function of the immunoglobulin heavy chain constant region comprises L234A, L235E, G237A, A330S, and P331S per EU numbering. In certain embodiments, the Fc domain comprises an alteration to one or more amino acid residues that alters binding of the antibody to the neonatal Fc receptor (FcRn). In certain embodiments, the alteration to one or more amino acid residues that alters binding of the antibody to the neonatal Fc receptor (FcRn) is to an amino acid residue selected from the list consisting of: I253A, I253D, I253P, S254A, H310A, H310D, H310E, H310Q, H435A, H435Q, Y436A, and combinations thereof per EU numbering. In certain embodiments, the alteration to one or more amino acid residues that alters binding of the antibody to the neonatal Fc receptor (FcRn) is to an amino acid residue selected from the list consisting of: I253A, S254A, H310A, H435Q, Y436A and combinations thereof per EU numbering. In certain embodiments, the alteration to one or more amino acid residues that alters binding of the antibody to the neonatal Fc receptor (FcRn) is to an amino acid residue selected from the list consisting of: I253A, H310A, H435Q, and combinations thereof per EU numbering. In certain embodiments, the alteration to one or more amino acid residues that alters binding of the antibody to the neonatal Fc receptor (FcRn) comprises I253A per EU numbering. In certain embodiments, the alteration to one or more amino acid residues that alters binding of the antibody to the neonatal Fc receptor (FcRn) comprises H310A per EU numbering. In certain embodiments, the alteration to one or more amino acid residues that alters binding of the antibody to the neonatal Fc receptor (FcRn) comprises H435Q per EU numbering. In certain embodiments, the antibody comprises a second antigen-binding domain and the antibody is a multivalent antibody In certain embodiments, the antibody comprises a molecular weight less than about 100,000 Daltons. In certain embodiments, the antibody comprises a molecular weight less than about 90,000 Daltons. In certain embodiments, the antibody comprises a molecular weight less than about 80,000 Daltons. In certain embodiments, the antibody comprises a molecular weight between about 60,000 Daltons and 110,000 Daltons. In certain embodiments, the antigen comprises FOLR1. In certain embodiments, the antigen-binding domain binds FOLR1. In certain embodiments, the antigen-binding domain comprises: a complementarity determining region (CDR) 1 comprising the amino acid sequence as set forth in SEQ ID NO: 1; a complementarity determining region (CDR) 2 comprising the amino acid sequence as set forth in SEQ ID NO: 2; and a complementarity determining region (CDR) 3 comprising the amino acid sequence as set forth in SEQ ID NO: 3. In certain embodiments, the first antigen-binding domain or the second antigen-binding domain comprise an amino acid sequence at least about 85%, 90%, 95%, 97%, 98%, 99%, or 100% identical to that set forth in SEQ ID NO: 4.

Also described herein is a method of delivering an antibody to a kidney of an individual, the method comprising: administering the antibody described herein to the individual.

Also described herein is a method of imaging an antigen within a kidney of an individual, the method comprising: administering an antibody of this disclosure to the individual.

A method of treating a kidney disease or disorder, the method comprising: administering an antibody of this disclosure to the individual. In certain embodiments, cells within the kidney express FOLR1. In certain embodiments, the individual is a human individual. In certain embodiments, the kidney disease or disorder is BK virus nephritis or nephropathy.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A and 1B show binding of anti-HER2 and anti-DLL3 VHH-Fc constructs.

FIGS. 2A, 2B, and 2C show binding of anti-HER2 and anti-DLL3 VHH-Fc constructs to cells expressing HER2 and/or DLL3.

FIGS. 3A and 3B show internalization of anti-HER2 and anti-DLL3 VHH-Fc constructs in cells expressing HER2 and DLL3.

FIG. 4 shows self-interaction data for anti-HER2 and anti-DLL3 VHH-Fc constructs.

FIG. 5 shows a diagram for chemical synthesis of linker molecules.

FIG. 6 shows a diagram for chemical synthesis of linker molecules.

FIGS. 7A, 7B, and 7C shows the immunoreactive fraction of different VHH-Fc constructs.

FIG. 8 shows a comparison of imaging with 111In labeled VHH-Fc compared to biodistribution of 225Ac labeled VHH-Fc.

FIGS. 9A, 9B, 9C, and 9D show biodistribution over time for labeled anti-HER2 VHH-Fc constructs.

FIGS. 10A, 10B and 10C show tumor:non-tumor tissue ratios for labeled anti-HER2 VHH-Fc constructs.

FIG. 11 shows biodistribution for labeled anti-HER2 VHH-Fc constructs.

FIG. 12 shows whole body clearance of VHH-Fc (H101) and VHH-Fc variants (H105, H107, and H108) labeled with 111In.

FIG. 13 shows biodistribution over time for labeled anti-DLL3 VHH-Fc constructs.

FIG. 14 shows biodistribution for labeled anti-DLL3 VHH-Fc constructs.

FIGS. 15A and 15B show biodistribution for 225Ac labeled anti-HER2 (15A) and anti-DLL3 (15B) VHH-Fc constructs.

FIGS. 16A, 16B, and 16C show the results of a toxicity study carried out with 225Ac labeled anti-HER2 VHH-Fc constructs.

FIG. 17 shows the immunoreactive fraction of different anti-DDL3 VHH-Fc constructs loaded with 177Lu.

FIG. 18 shows the chemical Structures of certain linker chelators described herein.

FIGS. 19A and 19B show a schematic of multivalent antibody formats.

FIG. 20 shows FOLR1 and DLL3 monospecific and bispecific multivalent formats.

FIGS. 21A to 21D show the tumor localization in mice 72-hours post administration

FIGS. 22A and 22B show biodistribution of human (VHH17) and mouse cross-reactive VHH-Fc (VHH19).

FIG. 23 shows binding of antibody constructs to cells expressing FOLR1.

FIG. 24 shows kidney uptake and retention of antibody constructs.

FIGS. 25A and 25B show increased kidney biodistribution of antibody constructs compared to other tissues.

### DETAILED DESCRIPTION

Provided herein are antibodies that are useful for targeting antigens within the kidney. In certain instances, the antibodies described herein (e.g., less than 150,000 Daltons) are advantageous in that they are capable of localizing to the kidney and binding targets within the by the kidney. Furthermore, the antibodies described herein do not undergo rapid renal excretion. In other instances, the antibodies described herein (e.g., less than 150,000 Daltons) are advantageous in that they are capable of localizing to the salivary glands and binding targets within the by the salivary glands. Although reducing reduce serum half-life or effector cell function is generally not associated with improved antibody efficacy, the antibodies described herein can achieve improved safety while being able effectively target the kidney and engage target antigens within the kidney.

### Antibodies

Provided herein are antibodies and immunoconjugates useful for targeting antigens present within the kidney, wherein the antibodies bind one or more antigens present within the kidney. In some embodiments, provided herein are antibodies comprising a polypeptide comprising an antigen-binding domain (e.g., an immunoglobulin single-chain domain), wherein the antigen-binding domain binds an antigen present within the kidney (e.g., FOLR1). In certain embodiments, the polypeptide further comprises an Fc domain.

In some embodiments, provided are antibody comprising a polypeptide having the structure:

A-C
wherein: A comprises an antigen-binding domain that binds to an antigen present within a kidney or salivary glands; and C comprises an Fc domain. In certain embodiments, the antibody comprises a homodimer of the polypeptide.

In certain embodiments, the antibody binds FOLR1. In certain embodiments, the antigen is FOLR1. In certain embodiments, the antibody binds BK virus. In certain embodiments, the antigen is a BK virus antigen.

In certain embodiments, the first antigen-binding domain and second antigen-binding domain each comprise a single-chain variable domain polypeptide selected from the group consisting of a scFv, VH, VL, VHH, and VNAR. In certain embodiments, the first antigen-binding domain and second antigen-binding domain each comprise an immunoglobulin single-chain variable domain polypeptide. In certain embodiments, the immunoglobulin single-chain variable domain polypeptide is selected from the group consisting of a VH, VL, and VHH. In certain embodiments, the first antigen-binding domain and second antigen-binding domain each comprise a VHH (e.g., a first VHH domain and a second VHH domain).

In certain embodiments, the Fc domain comprises an alteration to one or more amino acid residues that modulates (e.g., reduces, inhibits, decreases, prevents, etc.) an effector function of the Fc domain. In certain embodiments, the Fc domain comprises an alteration to one or more amino acid residues that alters (e.g., reduces, inhibits, decreases, prevents, etc.) serum half-life of the antibody. In certain embodiments, the Fc domain comprises an alteration to one or more amino acid residues that alters (e.g., reduces, inhibits, decreases, prevents, etc.) binding of the antibody to the neonatal Fc receptor (FcRn). In certain embodiments, the Fc domain comprises an alteration to one or more amino acid residues that modulates (e.g., reduces, inhibits, decreases, prevents, etc.) both (i) an effector function of the Fc domain (e.g., ADCC and/or CDC) and (ii) binding of the antibody to the neonatal Fc receptor (FcRn).

In certain embodiments, the molecule weight of the antibody is less than 150,000 Daltons. In certain embodiments, the molecule weight of the antibody is less than 120,000 Daltons. In certain embodiments, the molecule weight of the antibody is less than 110,000 Daltons. In certain embodiments, the molecule weight of the antibody is less than 100,000 Daltons. In certain embodiments, the molecule weight of the antibody is less than 90,000 Daltons. In certain embodiments, the molecule weight of the antibody is less than 80,000 Daltons. In certain embodiments, the molecule weight of the antibody is less than 75,000 Daltons. In certain embodiments, the molecular weight of the antibody is about 65,000 Daltons to about 130,000 Daltons. In certain embodiments, the molecular weight of the antibody is at least about 65,000 Daltons. In certain embodiments, the molecular weight of the antibody is at most about 130,000 Daltons. In certain embodiments, the molecular weight of the antibody is about 65,000 Daltons to about 70,000 Daltons, about 65,000 Daltons to about 75,000 Daltons, about 65,000 Daltons to about 80,000 Daltons, about 65,000 Daltons to about 90,000 Daltons, about 65,000 Daltons to about 100,000 Daltons, about 65,000 Daltons to about 110,000 Daltons, about 65,000 Daltons to about 120,000 Daltons, about 65,000 Daltons to about 130,000 Daltons, about 70,000 Daltons to about 75,000 Daltons, about 70,000 Daltons to about 80,000 Daltons, about 70,000 Daltons to about 90,000 Daltons, about 70,000 Daltons to about 100,000 Daltons, about 70,000 Daltons to about 110,000 Daltons, about 70,000 Daltons to about 120,000 Daltons, about 70,000 Daltons to about 130,000 Daltons, about 75,000 Daltons to about 80,000 Daltons, about 75,000 Daltons to about 90,000 Daltons, about 75,000 Daltons to about 100,000 Daltons, about 75,000 Daltons to about 110,000 Daltons, about 75,000 Daltons to about 120,000 Daltons, about 75,000 Daltons to about 130,000 Daltons, about 80,000 Daltons to about 90,000 Daltons, about 80,000 Daltons to about 100,000 Daltons, about 80,000 Daltons to about 110,000 Daltons, about 80,000 Daltons to about 120,000 Daltons, about 80,000 Daltons to about 130,000 Daltons, about 90,000 Daltons to about 100,000 Daltons, about 90,000 Daltons to about 110,000 Daltons, about 90,000 Daltons to about 120,000 Daltons, about 90,000 Daltons to about 130,000 Daltons, about 100,000 Daltons to about 110,000 Daltons, about 100,000 Daltons to about 120,000 Daltons, about 100,000 Daltons to about 130,000 Daltons, about 110,000 Daltons to about 120,000 Daltons, about 110,000 Daltons to about 130,000 Daltons, or about 120,000 Daltons to about 130,000 Daltons. In certain embodiments, the molecular weight of the antibody is about 65,000 Daltons, about 70,000 Daltons, about 75,000 Daltons, about 80,000 Daltons, about 90,000 Daltons, about 100,000 Daltons, about 110,000 Daltons, about 120,000 Daltons, or about 130,000 Daltons. In some embodiments, the molecular weight of the antibody is greater than about 60 Daltons.

### Multivalent Antibodies

Also provided herein are antibodies comprising a multivalent (e.g., tetravalent or greater) polypeptide (e.g., a multivalent antibody or antibody-derived polypeptide). In some embodiments, the multivalent antibodies are tetravalent. In certain embodiments, the multivalent antibodies are monospecific (e.g., binding only FOLR1 or DLL3). In certain embodiments, the multivalent antibodies are bispecific (e.g., binding both FOLR1 and DLL3). In some embodiments, the multivalent antibody comprises a molecular weigh less than 150,000 Daltons. In certain embodiments, the multivalent antibody of the antibody comprises a molecular weight less than 110,000 Daltons.

In some embodiments, provided are antibodies comprising multivalent binding domains, wherein the multivalent antibody comprises a polypeptide comprising: a first antigen-binding domain; and a second antigen-binding domain. In certain embodiments, the polypeptide further comprises an Fc domain.

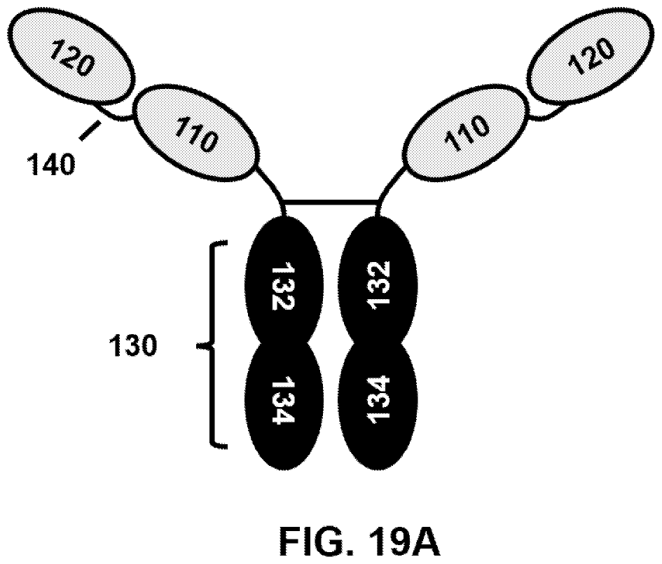

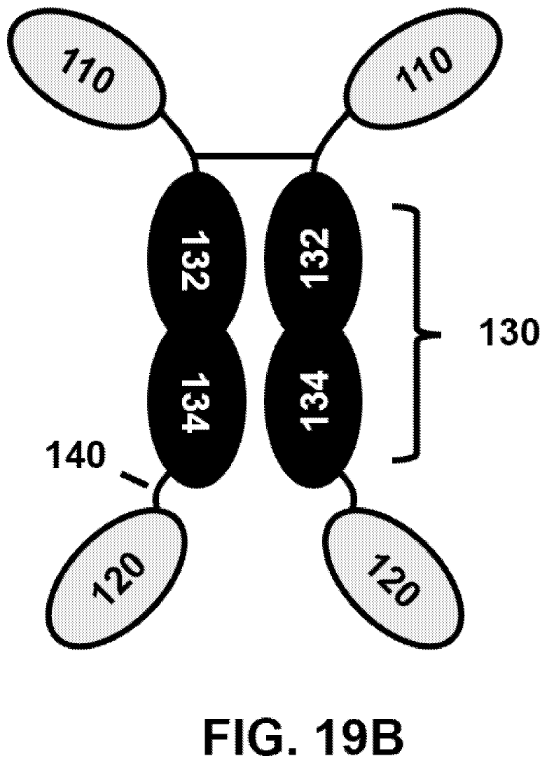

FIGS. 19A-B show exemplary multivalent antibody formats described herein. 110 depicts a first antigen-binding domain (e.g., an immunoglobulin single-chain domain). 120 depicts a second antigen-binding domain (e.g., an immunoglobulin single-chain domain). 130 depicts an Fc domain comprising a CH2-CH3 (132 and 134, respectively). 140 depicts an optional linker polypeptide.

In some embodiments, provided are comprising a multivalent antibody, wherein the multivalent antibody comprises a polypeptide having the structure of Formula I:

A-B-C
wherein: A comprises a first antigen-binding domain; B comprises a second antigen-binding domain; and C comprises an Fc domain.

In some embodiments, provided are a multivalent antibody, wherein the multivalent antibody comprises a polypeptide having the structure of Formula II:

A-C-B
wherein: A comprises a first antigen-binding domain; B comprises a second antigen-binding domain; and C comprises an Fc domain.

In some embodiments, provided is a multivalent antibody, wherein the multivalent antibody comprises a polypeptide having the structure of Formula III:

A-L1-B-L2-C
wherein: A comprises a first antigen-binding domain; B comprises a second antigen-binding domain; C comprises an Fc domain; L1 is a polypeptide linker (e.g., a poly GS linker); and L2 is a polypeptide linker (e.g., identical to or different from L1).

In some embodiments, provided is a multivalent antibody wherein the multivalent antibody comprises a polypeptide having the structure of Formula IV:

A-L1-C-L2-B
wherein: A comprises a first antigen-binding domain; B comprises a second antigen-binding domain; C comprises an Fc domain; L1 is a polypeptide linker (e.g., a poly GS linker); and L2 is a polypeptide linker (e.g., identical to or different from L1).

In certain embodiments, the multivalent antibody comprises a homodimer of the polypeptide (e.g., mediated through Fc domain dimerization). In certain embodiments, the multivalent antibody comprises a molecular weight less than 150,000 Daltons. In certain embodiments, the multivalent antibody comprises a molecular weight less than 140,000 Daltons. In certain embodiments, the multivalent antibody comprises a molecular weight less than 130,000 Daltons. In certain embodiments, the multivalent antibody comprises a molecular weight less than 120,000 Daltons. In certain embodiments, the multivalent antibody comprises a molecular weight less than 110,000 Daltons.

In certain embodiments, the first antigen-binding domain and second antigen-binding domain each comprise a single-chain variable domain polypeptide selected from the group consisting of a scFv, VH, VL, VHH, and VNAR. In certain embodiments, the first antigen-binding domain and second antigen-binding domain each comprise an immunoglobulin single-chain variable domain polypeptide. In certain embodiments, the immunoglobulin single-chain variable domain polypeptide is selected from the group consisting of a VH, VL, and VHH. In certain embodiments, the first antigen-binding domain and second antigen-binding domain each comprise a VHH (e.g., a first VHH domain and a second VHH domain).

In certain embodiments, the Fc domain comprises an alteration to one or more amino acid residues that modulates (e.g., reduces, inhibits, decreases, prevents, etc.) an effector function of the Fc domain. In certain embodiments, the Fc domain comprises an alteration to one or more amino acid residues that alters (e.g., reduces, inhibits, decreases, prevents, etc.) serum half-life of the antibody. In certain embodiments, the Fc domain comprises an alteration to one or more amino acid residues that alters (e.g., reduces, inhibits, decreases, prevents, etc.) binding of the antibody to the neonatal Fc receptor (FcRn). In certain embodiments, the Fc domain comprises an alteration to one or more amino acid residues that modulates (e.g., reduces, inhibits, decreases, prevents, etc.) both (i) an effector function of the Fc domain (e.g., ADCC and/or CDC) and (ii) binding of the antibody to the neonatal Fc receptor (FcRn).

In certain embodiments, the multivalent antibody is a monospecific (e.g., binding only FOLR1) for a kidney antigen. In certain embodiments, the first antigen-binding domain and the second antigen-binding domain bind FOLR1.

In certain embodiments, the multivalent antibody is a bispecific (e.g., binding FOLR1 and another antigen). In certain embodiments, the first antigen-binding domain binds FOLR1.

### Antigen-Binding Domains

A “variable region” or “variable domain” refers to and encompasses the domain of an antibody heavy or light chain or immunoglobulin single-chain variable domain (e.g., VHH) antibody that is involved in binding the antibody to an antigen. The variable domains of the heavy chain, light chain (VH and VL, respectively), or VHH of a native antibody generally have similar structures, with each domain comprising four conserved framework regions (FRs) and three hypervariable regions (HVRs). (See, e.g., Kindt et al. Kuby Immunology, 6th ed., W.H. Freeman and Co. (2007). A single VH or VL domain may be sufficient to confer antigen-binding specificity. A single VHH is sufficient to confer antigen-binding specificity.

A “immunoglobulin single-chain variable domain” or “immunoglobulin single-chain variable domain antibody” or “immunoglobulin single variable domain” or “single chain antibpody” or “VHH”, interchangeably used herein, refers to and encompasses immunoglobulin molecules wherein the antigen binding site is present on, and formed by, a single immunoglobulin domain (e.g., variable domain).

Typically, in conventional immunoglobulins, a heavy chain variable domain (VH) and a light chain variable domain (VL) interact to form an antigen binding site, i.e., providing a total of 6 CDRs for antigen binding site formation. In this case, the complementarity determining regions (CDRs) of both VH and VL can contribute to the antigen. The antigen-binding domain of a conventional antibody (such as an IgG, IgM, IgA, IgD or IgE molecule having a cognate VL and VH), of a Fab fragment, a F(ab′)2 fragment, an Fv fragment such as a disulphide linked Fv, a scFv fragment, or a diabody derived from such conventional 4-chain antibody, is distinct from a single chain variable domain antibody.

A VH, VL, or VHH region can be subdivided into regions of hypervariability, termed “complementarity determining regions” (CDR), interspersed with regions that are more conserved, termed “framework regions” (FR or FW).

The extent of the framework region and CDRs can defined by a number of methods (see, Kabat, E. A., et al. (1991) Sequences of Proteins of Immunological Interest, Fifth Edition, U.S. Department of Health and Human Services, NIH Publication No.91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme). Methods encompassed and included herein Chothia, AbM, Kabat, Contact, and/or IMGT.

A “complementarity determining region” or “CDR” refers to and encompasses the sequences of amino acids within antibody variable regions which confer antigen specificity and binding affinity. In general, there are three CDRs in each heavy chain variable region (HCDR1, HCDR2, and HCDR3) and three CDRs in each light chain variable region (LCDR1, LCDR2, and LCDR3). An immunoglobulin single-chain variable domain antibody comprises 3 CDRs (CDR1, CDR2, and CDR3).

The structure of an immunoglobulin single variable domain sequence can be considered to be comprised of four framework regions (“FRs”), which are referred to in the art and herein as “Framework region 1” (“FR1”); as “Framework region 2” (“FR2”); as “Framework region 3” (“FR3”); and as “Framework region 4” (“FR4”), respectively; which framework regions are interrupted by three complementary determining regions (“CDRs”), which are referred to in the art and herein as “Complementarity Determining Region 1” (“CDR1”); as “Complementarity Determining Region 2” (“CDR2”); and as “Complementarity Determining Region 3” (“CDR3”), respectively. As such, the single variable domain may be a light chain variable domain sequence (e.g., a VL-sequence) or a suitable fragment thereof; or a heavy chain variable domain sequence (e.g., a VH-sequence or VHH sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit). An immunoglobulin single-chain variable domain can for example be a heavy chain ISVD, such as a VH, VHH, including a camelized VH or humanized VHH. Preferably, it is a VHH, including a camelized VH or humanized VHH.

“VHH domains”, also known as VHHs, VHH antibody fragments, and VHH antibodies, have originally been described as the antigen binding immunoglobulin variable domain of “heavy chain antibodies” (i.e., of “antibodies devoid of light chains”; Hamers-Casterman et al. Nature 363: 446-448, 1993). The term “VHH domain” and “immunoglobulin single-chain variable domain” is used to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHH's, reference is made to the review article by Muyldermans (Reviews in Molecular Biotechnology 74: 277-302, 2001).

A “humanized VHH” comprises an amino acid sequence that corresponds to the amino acid sequence of a naturally occurring VHH domain, but that has been “humanized”, i.e. by replacing one or more amino acid residues in the amino acid sequence of said naturally occurring VHH sequence (and in particular in the framework sequences) by one or more of the amino acid residues that occur at the corresponding position(s) in a VH domain from a conventional 4-chain antibody from a human being (e.g. indicated above). This can be performed in a manner known per se, which will be clear to the skilled person, for example on the basis of the further description herein and the prior art (e.g. WO 2008/020079). Again, it should be noted that such humanized VHHS can be obtained in any suitable manner known per se and thus are not strictly limited to polypeptides that have been obtained using a polypeptide that comprises a naturally occurring VHH domain as a starting material.

Affinity encompasses and/or refers to the strength of the sum total of noncovalent interactions between a single binding site of a molecule (e.g., an antibody) and its binding partner (e.g., an antigen). Unless indicated otherwise, as used herein, binding affinity encompasses and refers to intrinsic binding affinity which reflects a 1:1 interaction between members of a binding pair (e.g., antibody and antigen). The affinity of a molecule X for its partner Y can be represented by the dissociation constant (KD). Affinity can be measured by common methods known in the art, including those described herein. Specific illustrative and exemplary embodiments for measuring binding affinity are described throughout.

An affinity matured antibody encompasses and/or refers to an antibody with one or more alterations in one or more hypervariable regions (HVRs), compared to a parent antibody which does not possess such alterations, such alterations resulting in an improvement in the affinity of the antibody for antigen.

Binding and a determination of binding can be readily determined by methods known within the art (e.g., ELISA, surface plasmon resonance, bio-layer interferometry, isothermal calorimetry, etc.). In some embodiments, binding is determined by ELISA. In some embodiments, binding comprising a KD less than, e.g., 10\hat{ }−5 M (10 uM) as measured by surface plasmon resonance, bio-layer interferometry, or isothermal calorimetry. In some embodiments, binding comprising a KD less than, e.g., 10\hat{ }−6 M (1 uM) surface plasmon resonance, bio-layer interferometry, or isothermal calorimetry. In some embodiments, binding comprising a KD less than, e.g., 10\hat{ }−7 M (100 nM) surface plasmon resonance, bio-layer interferometry, or isothermal calorimetry.

In certain embodiments, the antibody comprises one or more naturally occurring amino acids. In certain embodiments, the antibody consists of naturally occurring amino acids. As used herein, naturally occurring amino acids include and/or refer to amino acids which are found in nature and are not manipulated by man. In certain instances, naturally occurring includes and/or further refers to the 20 conventional amino acids: alanine (A or Ala), cysteine (C or Cys), aspartic acid (D or Asp), glutamic acid (E or Glu), phenylalanine (F or Phe), glycine (G or Gly), histidine (H or His), isoleucine (I or Ile), lysine (K or Lys), leucine (L or Leu), methionine (M or Met), asparagine (N or Asn), proline (P or Pro), glutamine (Q or Gln), arginine (R or Arg), serine (S or Ser), threonine (T or Thr), valine (V or Val), tryptophan (W or Trp), and tyrosine (Y or Tyr).

In some embodiments, the antibody comprises a variant sequence of the antibody. In certain instances, amino acid substitutions can be made in the sequence of any of the antibodies described herein, without necessarily decreasing or ablating its activity (as measured by, e.g., the binding or functional assays described herein). Accordingly, in some embodiments, the variant sequence comprises one or more amino acid substitutions (e.g., within the variable region or within one or more CDRs). In some embodiments, the variant sequence comprises one or more substitutions in one or more CDRs. In certain embodiments, the variant sequence comprises one amino acid substitution. In certain embodiments, the variant sequence comprises two amino acid substitutions. In certain embodiments, the variant sequence comprises three amino acid substitutions. In certain instances, substitutions include conservative substitutions (e.g., substitutions with amino acids of comparable chemical characteristics). In certain instances, a non-polar amino acid can be substituted and replaced with another non-polar amino acid, wherein non-polar amino acids include alanine, leucine, isoleucine, valine, glycine, proline, phenylalanine, tryptophan and methionine. In certain instances, a neutrally charged polar amino acids can be substituted and replaced with another neutrally charged polar amino acid, wherein neutrally charged polar amino acids include serine, threonine, cysteine, tyrosine, asparagine, and glutamine. In certain instances, a positively charged amino acid can be substituted and replaced with another positively charged amino acid, wherein positively charged amino acids include arginine, lysine and histidine. In certain instances, a negatively charged amino acid can be substituted and replaced with another negatively charged amino acid, wherein negatively charged amino acids include aspartic acid and glutamic acid. Examples of amino acid substitutions also include substituting an L-amino acid for its corresponding D-amino acid, substituting cysteine for homocysteine or other non-natural amino acids.

In certain embodiments, the antibody comprises one or more non-natural amino acids. In certain embodiments, the antibody consists of non-natural amino acids. As used herein, non-natural amino acids and/or unnatural amino acids include and/or refer to amino acid structures that cannot be generated biosynthetically in any organism using unmodified or modified genes from any organism. For example, these include, but are not limited to, modified amino acids and/or amino acid analogues that are not one of the 20 naturally occurring amino acids (e.g., non-natural side chain variant sequence amino acids), D-amino acids, homo amino acids, beta-homo amino acids, N-methyl amino acids, alpha-methyl amino acids, or. By way of further example, non-natural amino acids also include 4-Benzoylphenylalanine (Bpa), Aminobenzoic Acid (Abz), Aminobutyric Acid (Abu), Aminohexanoic Acid (Ahx), Aminoisobutyric Acid (Aib), Citrulline (Cit), Diaminobutyric Acid (Dab), Diaminopropanoic Acid (Dap), Diaminopropionic Acid (Dap), Gamma-Carboxyglutamic Acid (Gla), Homoalanine (Hala), Homoarginine (Harg), Homoasparagine (Hasn), Homoaspartic Acid (Hasp), Homocysteine (Hcys), Homoglutamic Acid (Hglu), Homoglutamine (Hgln), Homoisoleucine (Hile), Homoleucine (Hleu), Homomethionine (Hmet), Homophenylalanine (Hphe), Homoserine (Hser), Homotyrosine (Htyr), Homovaline (Hval), Hydroxyproline (Hyp), Isonipecotic Acid (Inp), N aphthylalanine (Nal), Nipecotic Acid (Nip), Norleucine (Nle), Norvaline (Nva), Octahydroindole-2-carboxylic Acid (Oic), Penicillamine (Pen), Phenylglycine (Phg), Pyroglutamic Acid (Pyr), Sarcosine (Sar), tButylglycine (Tle), and Tetrahydro-isoquinoline-3-carboxylic Acid (Tic). Such non-natural amino acid residues can be introduced by substitution of naturally occurring amino acids, and/or by insertion of non-natural amino acids into the naturally occurring antibody sequence. A non-natural amino acid residue also can be incorporated such that a desired functionality is imparted to the apelin molecule, for example, the ability to link a functional moiety (e.g., PEG).

A stable formulation refers to and/or encompasses a formulation wherein the protein (e.g., antibody) therein essentially retains its physical stability and/or chemical stability and/or biological activity upon storage at an intended storage temperature, e.g., 2-8° C. In some embodiments, the formulation essentially retains its physical and chemical stability, as well as its biological activity upon storage. A storage period can be selected based on the intended shelf-life of the formulation. Furthermore, the formulation is stable following freezing (to, e.g., −20° C.) and thawing of the formulation, for example following 1 or more cycles of freezing and thawing. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993), for example. Stability can be measured at a selected temperature for a selected time period. Stability can be evaluated qualitatively and/or quantitatively in a variety of different ways, including evaluation of aggregate formation (for example using size exclusion chromatography, by measuring turbidity, and/or by visual inspection); by assessing charge heterogeneity using cation exchange chromatography or capillary zone electrophoresis; SDS-PAGE analysis to compare reduced and intact antibody; evaluating biological activity or antigen binding function of the antibody; and the methods described herein. Instability can involve any one or more of: aggregation, deamidation (e.g., Asn deamidation), oxidation (e.g. Met oxidation), isomerization (e.g. Asp isomeriation), clipping/hydrolysis/fragmentation (e.g. hinge region fragmentation), succinimide formation, unpaired cysteine(s), etc.

A pharmaceutically acceptable carrier encompasses and/or refers to an ingredient in a pharmaceutical formulation, other than an active ingredient, which is nontoxic to a subject. A pharmaceutically acceptable carrier encompasses, but is not limited to, a buffer, excipient, stabilizer, or preservative.

A polypeptide or protein are used interchangeably, and encompass and/or refer to a polymer of amino acid residues and are not limited to a minimum length. Polypeptides, including the provided antibodies and antibody chains and other peptides, e.g., linkers and binding peptides, can include amino acid residues including natural and/or non-natural amino acid residues. The terms also include post-expression modifications of the polypeptide, for example, glycosylation, sialylation, acetylation, phosphorylation, and the like. In some embodiments, the polypeptides can contain modifications with respect to a native or natural sequence, as long as the protein maintains the desired activity. These modifications can be deliberate, as through site-directed mutagenesis, or can be accidental, such as through mutations of hosts which produce the proteins or errors due to PCR amplification.

The determination of percent identity or percent similarity between two sequences can be accomplished using a mathematical algorithm. A non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul et al., 1990, J. Mol. Biol. 215:403-410. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. Additional algorithms for sequence analysis are known in the art and include ADVANCE and ADAM as described in Torellis and Robotti, 1994, Comput. Appl. Biosci. 10:3-5; and FASTA described in Pearson and Lipman, 1988, Proc. Natl. Acad. Sci. USA 85:2444-8. Alternatively, sequence alignment may be carried out using the CLUSTAL algorithm (e.g., as provided in the program Clustal-omega), as described by Higgins et al., 1996, Methods Enzymol. 266:383-402.

As used herein the term individual, patient, or subject includes and/or refers to individuals diagnosed with, suspected of being afflicted with, or at-risk of developing at least one disease, condition, or status for which the described compositions and method are useful for treating. In certain embodiments, the individual is a mammal. In certain embodiments, the mammal is a mouse, rat, rabbit, dog, cat, horse, cow, sheep, pig, goat, llama, alpaca, or yak. In certain embodiments, the individual is a human.

The antibodies described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a genomic region. In certain embodiments, the expression vector is a plasmid. In certain embodiments, the expression vector is a lentivirus, adenovirus, or adeno-associated virus. In certain embodiments, the expression vector is an adenovirus. In certain embodiments, the expression vector is an adeno-associated virus. In certain embodiments, the expression vector is a lentivirus.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application.

The nucleic acids encoding the antibodies described herein can be used to infect, transfect, transform, or otherwise render a suitable cell transgenic for the nucleic acid, thus enabling the production of antibodies for commercial or therapeutic uses. Standard cell lines and methods for the production of antibodies from a large-scale cell culture are known in the art. See e.g., Li et al., “Cell culture processes for monoclonal antibody production.” Mabs. 2010 September-October; 2(5): 466-477. In certain embodiments, the cell is a Eukaryotic cell. In certain embodiments, the Eukaryotic cell is a mammalian cell. In certain embodiments, the mammalian cell is a cell line useful for producing antibodies is a Chines Hamster Ovary cell (CHO) cell, an NSO murine myeloma cell, or a PER.C6® cell. In certain embodiments, the nucleic acid encoding the antibody is integrated into a genomic locus of a cell useful for producing antibodies. In certain embodiments, described herein is a method of making an antibody comprising culturing a cell comprising a nucleic acid encoding an antibody under conditions in vitro sufficient to allow production and secretion of said antibody.

In certain embodiments, described herein, is a master cell bank comprising: (a) a mammalian cell line comprising a nucleic acid encoding an antibody described herein integrated at a genomic location; and (b) a cryoprotectant. In certain embodiments, the cryoprotectant comprises glycerol or DMSO. In certain embodiments, the master cell bank comprises: (a) a CHO cell line comprising a nucleic acid encoding an antibody of the disclosure; and (b) a cryoprotectant. In certain embodiments, the cryoprotectant comprises glycerol or DMSO. In certain embodiments, the master cell bank is contained in a suitable vial or container able to withstand freezing by liquid nitrogen.

Also described herein are methods of making an antibody described herein. Such methods comprise incubating a cell or cell-line comprising a nucleic acid encoding the antibody in a cell culture medium under conditions sufficient to allow for expression and secretion of the antibody, and further harvesting the antibody from the cell culture medium. The harvesting can further comprise one or more purification steps to remove live cells, cellular debris, non-antibody proteins or polypeptides, undesired salts, buffers, and medium components. In certain embodiments, the additional purification step(s) include centrifugation, ultracentrifugation, protein A, protein G, protein A/G, or protein L purification, and/or ion exchange chromatography.

“Treat,” “treatment,” or “treating,” as used herein refers to, e.g., a deliberate intervention to a physiological disease state resulting in the reduction in severity of a disease or condition; the reduction in the duration of a condition course; the amelioration or elimination of one or more symptoms associated with a disease or condition; or the provision of beneficial effects to a subject with a disease or condition. Treatment does not require curing the underlying disease or condition.

A “therapeutically effective amount,” “effective dose,” “effective amount,” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, “pharmaceutically acceptable” with reference to a carrier” “excipient” or “diluent” includes any and all solvents, dispersion media, coatings, antibacterial and antifungal agents, isotonic and absorption delaying agents, and the like that are physiologically compatible. In some embodiments, the carrier is suitable for intravenous, intramuscular, subcutaneous, parenteral, spinal or epidermal administration (e.g., by injection or infusion). Depending on the route of administration, the active compound, i.e., antibody, can be coated in a material to protect the compound from the action of acids and other natural conditions that can inactivate the compound.

The pharmaceutical compounds described herein can include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66: 1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

As used herein, treatment or treating include and/or refer to a pharmaceutical or other intervention regimen used for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made. Skilled artisans will recognize that given a population of potential individuals for treatment not all will respond or respond equally to the treatment. Such individuals are considered treated.

Typically, the generation of immunoglobulins involves the immunization of experimental animals, fusion of immunoglobulin producing cells to create hybridomas and screening for the desired specificities. Alternatively, immunoglobulins can be generated by screening of naive or synthetic libraries e.g. by phage display. The generation of immunoglobulin sequences, such as VHHs and immunoglobulin single-chain variable domain, has been described in various publications, among which WO 94/04678, Hamers-Casterman et al. 1993 and Muyldermans et al. 2001 (Reviews in Molecular Biotechnology 74: 277-302, 2001) can be exemplified. In these methods, camelids are immunized with the target antigen in order to induce an immune response against said target antigen. The repertoire of Nanobodies obtained from said immunization is further screened for Nanobodies that bind the target antigen. In these instances, the generation of antibodies requires purified antigen for immunization and/or screening. Antigens can be purified from natural sources, or during recombinant production.

The multivalent antibodies described herein comprise an antigen-binding domain. In certain instances, independent any antigen-binding domain having a molecular weigh less than about 25,000 Daltons (e.g., a VHH having a molecular weight of about 15,000 Daltons) can be used herein, wherein the multivalent antibody totals a molecular weight of less than 150,000 Daltons. In certain embodiments, an antigen-binding domain comprises molecular weight of less than about 25,000 Daltons. In certain embodiments, an antigen-binding domain comprises molecular weight of less than about 20,000 Daltons. In certain embodiments, an antigen-binding domain comprises molecular weight of less than about 19,000 Daltons. In certain embodiments, an antigen-binding domain comprises molecular weight of less than about 18,000 Daltons. In certain embodiments, an antigen-binding domain comprises molecular weight of less than about 17,000 Daltons. In certain embodiments, an antigen-binding domain comprises molecular weight of less than about 16,000 Daltons. In certain embodiments, an antigen-binding domain comprises molecular weight of less than about 15,000 Daltons.

In certain embodiments, an antigen-binding domain comprises molecular weight of about 12,000 Daltons to about 25,000 Daltons. In certain embodiments, an antigen-binding domain comprises molecular weight of at most about 25,000 Daltons. In certain embodiments, an antigen-binding domain comprises molecular weight of about 12,000 Daltons to about 13,000 Daltons, about 12,000 Daltons to about 14,000 Daltons, about 12,000 Daltons to about 15,000 Daltons, about 12,000 Daltons to about 16,000 Daltons, about 12,000 Daltons to about 17,000 Daltons, about 12,000 Daltons to about 18,000 Daltons, about 12,000 Daltons to about 19,000 Daltons, about 12,000 Daltons to about 20,000 Daltons, about 12,000 Daltons to about 25,000 Daltons, about 13,000 Daltons to about 14,000 Daltons, about 13,000 Daltons to about 15,000 Daltons, about 13,000 Daltons to about 16,000 Daltons, about 13,000 Daltons to about 17,000 Daltons, about 13,000 Daltons to about 18,000 Daltons, about 13,000 Daltons to about 19,000 Daltons, about 13,000 Daltons to about 20,000 Daltons, about 13,000 Daltons to about 25,000 Daltons, about 14,000 Daltons to about 15,000 Daltons, about 14,000 Daltons to about 16,000 Daltons, about 14,000 Daltons to about 17,000 Daltons, about 14,000 Daltons to about 18,000 Daltons, about 14,000 Daltons to about 19,000 Daltons, about 14,000 Daltons to about 20,000 Daltons, about 14,000 Daltons to about 25,000 Daltons, about 15,000 Daltons to about 16,000 Daltons, about 15,000 Daltons to about 17,000 Daltons, about 15,000 Daltons to about 18,000 Daltons, about 15,000 Daltons to about 19,000 Daltons, about 15,000 Daltons to about 20,000 Daltons, about 15,000 Daltons to about 25,000 Daltons, about 16,000 Daltons to about 17,000 Daltons, about 16,000 Daltons to about 18,000 Daltons, about 16,000 Daltons to about 19,000 Daltons, about 16,000 Daltons to about 20,000 Daltons, about 16,000 Daltons to about 25,000 Daltons, about 17,000 Daltons to about 18,000 Daltons, about 17,000 Daltons to about 19,000 Daltons, about 17,000 Daltons to about 20,000 Daltons, about 17,000 Daltons to about 25,000 Daltons, about 18,000 Daltons to about 19,000 Daltons, about 18,000 Daltons to about 20,000 Daltons, about 18,000 Daltons to about 25,000 Daltons, about 19,000 Daltons to about 20,000 Daltons, about 19,000 Daltons to about 25,000 Daltons, or about 20,000 Daltons to about 25,000 Daltons. In certain embodiments, an antigen-binding domain comprises molecular weight of about 12,000 Daltons, about 13,000 Daltons, about 14,000 Daltons, about 15,000 Daltons, about 16,000 Daltons, about 17,000 Daltons, about 18,000 Daltons, about 19,000 Daltons, about 20,000 Daltons, or about 25,000 Daltons.

In certain embodiments, the antigen-binding domain comprises a single-chain variable domain polypeptide selected from the group consisting of a scFv, VH, VL, VHH, and VNAR. In certain embodiments, the antigen-binding domain comprises an immunoglobulin single-chain variable domain polypeptide. In certain embodiments, the immunoglobulin single-chain variable domain polypeptide is selected from the group consisting of a VH, VL, and VHH. In certain embodiments, In certain embodiments, the antigen-binding domain comprises a VHH (e.g., a first VHH domain and a second VHH domain).

For example, in certain embodiments, the first antigen-binding domain and second antigen-binding domain each comprise a single-chain variable domain polypeptide selected from the group consisting of a scFv, VH, VL, VHH, and VNAR. In certain embodiments, the first antigen-binding domain and second antigen-binding domain each comprise an immunoglobulin single-chain variable domain polypeptide. In certain embodiments, the immunoglobulin single-chain variable domain polypeptide is selected from the group consisting of a VH, VL, and VHH. In certain embodiments, the first antigen-binding domain and second antigen-binding domain each comprise a VHH (e.g., a first VHH domain and a second VHH domain).

Provided herein are antigen-binding domains that bind FOLR1. “FOLR1” or “Folate receptor alpha” or MOv18” or Folate Receptor 1” refers to and encompasses the protein encoded by the FOLR1 gene (see NC_000011.10 (72189709 . . . 72196323); NCBI Gene 2348, or UniProt ID P15328).

In some embodiments, the antigen-binding domain is an immunoglobulin single-chain domain comprising: a complementarity determining region (CDR) 1, a complementarity determining region (CDR) 2, and a complementarity determining region (CDR) 3 of SEQ ID NO: 4, wherein the CDR1-3 are defined using the Kabat definition. In some embodiments, the antigen-binding domain is an immunoglobulin single-chain domain comprising: a complementarity determining region (CDR) 1, a complementarity determining region (CDR) 2, and a complementarity determining region (CDR) 3 of SEQ ID NO: 4, wherein the CDR1-3 are defined using the Chothia definition. In some embodiments, the antigen-binding domain is an immunoglobulin single-chain domain comprising: a complementarity determining region (CDR) 1, a complementarity determining region (CDR) 2, and a complementarity determining region (CDR) 3 of SEQ ID NO: 4, wherein the CDR1-3 are defined using the AbM definition. In some embodiments, the antigen-binding domain is an immunoglobulin single-chain domain comprising: a complementarity determining region (CDR) 1, a complementarity determining region (CDR) 2, and a complementarity determining region (CDR) 3 of SEQ ID NO: 4, wherein the CDR1-3 are defined using the Contact definition. In some embodiments, the antigen-binding domain is an immunoglobulin single-chain domain comprising: a complementarity determining region (CDR) 1, a complementarity determining region (CDR) 2, and a complementarity determining region (CDR) 3 of SEQ ID NO: 4, wherein the CDR1-3 are defined using the IMGT definition.

In some embodiments, the antigen-binding domain is an immunoglobulin single-chain domain comprising: a complementarity determining region (CDR) 1 comprising the amino acid sequence as set forth in SEQ ID NO: 1; a complementarity determining region (CDR) 2 comprising the amino acid sequence as set forth in SEQ ID NO: 2; and a complementarity determining region (CDR) 3 comprising the amino acid sequence as set forth in SEQ ID NO: 3, wherein the immunoglobulin single-chain domain binds FOLR1.

In certain embodiments, the antigen-binding domain is an immunoglobulin single-chain domain comprising an amino acid sequence having at least 70% sequence identity to SEQ ID NO: 4. In certain embodiments, the antigen-binding domain is an immunoglobulin single-chain domain comprising an amino acid sequence having at least 80% sequence identity to SEQ ID NO: 4. In certain embodiments, the antigen-binding domain is an immunoglobulin single-chain domain comprising an amino acid sequence having at least 85% sequence identity to SEQ ID NO: 4. In certain embodiments, the antigen-binding domain is an immunoglobulin single-chain domain comprising an amino acid sequence having at least 90% sequence identity to SEQ ID NO: 4. In certain embodiments, the antigen-binding domain is an immunoglobulin single-chain domain comprising an amino acid sequence having at least 95% sequence identity to SEQ ID NO: 4. In certain embodiments, the antigen-binding domain is an immunoglobulin single-chain domain comprising an amino acid sequence having at least 97% sequence identity to SEQ ID NO: 4. In certain embodiments, the antigen-binding domain is an immunoglobulin single-chain domain comprising an amino acid sequence having at least 98% sequence identity to SEQ ID NO: 4. In certain embodiments, the antigen-binding domain is an immunoglobulin single-chain domain comprising an amino acid sequence having at least 99% sequence identity to SEQ ID NO: 4. In certain embodiments, the antigen-binding domain is an immunoglobulin single-chain domain comprising an amino acid sequence as set forth in SEQ ID NO: 4.

### Fc Domains

A “Fc region” (fragment crystallizable region) or “Fc domain” or “Fc” refers to and encompasses the C-terminal, non-antigen-binding region of the heavy chain of an antibody that mediates the binding of the immunoglobulin to host tissues or factors, including binding to Fc receptors located on various cells of the immune system (e.g., effector cells) or to the first component (Clq) of the classical complement system. An antibody constant region generally comprises the CL region (e.g., for light chains) or CH1-CH2-CH3 regions. Generally, an Fc domain generally refers to and encompasses the CH2-CH3 regions of a heavy chain constant region. For IgG, the Fc domain comprises immunoglobulin domains CH2 and CH3 (Cy2 and Cy3), and optionally all or a portion of the hinge region between CHI (Cy1) and CH2 (Cy2). In some embodiments, the Fc domain includes, from N- to C-terminus, CH2-CH3 and hinge-CH2-CH3. In some embodiments, the Fc domain is derived from IgG1, IgG2, IgG3 or IgG4, comprising the hinge-CH2-CH3 domains/regions. Additionally, in certain embodiments, wherein the Fc domain is a human IgG1 Fc domain, the hinge includes a C220S ammo acid substitution. Furthermore, in some embodiments where the Fc domain is a human IgG4 Fc domain, the hinge includes a S228P amino acid substitution. Although the boundaries of the Fc region may vary, the human IgG heavy chain Fc region is usually defined to include residues E216, C226, or A231 to its carboxyl-terminus, wherein the numbering is according to the EU. In some embodiments, as is more fully described below, amino acid modifications are made to the Fc region, for example to alter binding to one or more FcyR or to the FcRn. An Fc domain can be a native sequence Fc, including any allotypic variant, or a variant Fc (comprising one or more mutations that reduce effector cell function and/or FcRN).

A “hinge” or “hinge region” or “antibody hinge region” or “immunoglobulin hinge region” refers to and encompasses the flexible polypeptide comprising the amino acids between the first (CHi) and second (CH2) heavy chain constant domains of an antibody. Structurally, the IgG CHi domain ends at EU position 215, and the IgG CH2 domain begins at residue EU position 231. For IgG, the antibody hinge includes positions 216 (E216 in IgGl) to 230 (P230 in IgGl), wherein the numbering is according to the EU index as in Kabat. In some embodiments, for example in the context of an Fc region, the hinge (full length or a fragment of the hinge) is included, generally referring to positions 216-230.

An “isotype” refers to and encompasses the antibody class (e.g., IgG1, IgG2, IgG3, IgG4, IgM, IgAQ1, IgA2, IgD, and IgE antibody) that is encoded by the heavy chain constant domain genes. The full-length amino acid sequence of each wild-type human IgG constant region (including all domains, i.e., CHI domain, hinge, CH2 domain, and CH3 domain) is cataloged in the UniProt database available on-line, e.g., as P01857 (IgG1), P01859 (IgG2), P01860 (IgG3), and P01861 (IgG4), or different allotypes thereof. A domain of a heavy chain constant region is of an “IgG1 isotype,” “IgG2 isotype,” “IgG3 isotype,” or “IgG4 isotype,” if the domain comprises the amino acid sequence of the corresponding domain of the respective isotype, or a variant thereof (that has a higher homology to the corresponding domain of the respective isotype than it does to that of the other isotypes). An “allotype” refers to naturally occurring variants within a specific isotype group, which variants differ in a few amino acids (see, e.g., Jefferies et al. (2009) mAbs 1:1). In certain embodiments, the immunoglobulin heavy chain constant region is a human immunoglobulin heavy chain constant region. In certain embodiments, the immunoglobulin heavy chain constant region is an IgA, IgG1, IgG2, IgG3, or IgG4 isotype. In certain embodiments, the immunoglobulin heavy chain constant region is an IgG1 isotype. In certain embodiments, the immunoglobulin heavy chain constant region is an IgG4 isotype.

In some embodiments, the Fc domain comprises a variant Fc domain comprising one or mutations that modulate (e.g., reduce, inhibit, decrease, prevent, etc.) effector function associated with a heavy chain constant region, FcRn binding, or both.

The immunoglobulin heavy chain constant region can be a variant constant region that comprises one or more alterations to an amino acid residues that confers additional utility and advantageous properties to the antibodies described herein. In certain embodiments, the immunoglobulin heavy chain constant region comprises an alteration to one or more amino acid residues that reduces an effector function of the immunoglobulin heavy chain constant region or alters binding of the antibody to the neonatal Fc receptor (FcRn). In certain embodiments, the immunoglobulin heavy chain constant region comprises an alteration to one or more amino acid residues that reduces an effector function of the immunoglobulin heavy chain constant region or reduces binding of the antibody to the neonatal Fc receptor (FcRn). In certain embodiments, the immunoglobulin heavy chain constant region comprises an alteration to one or more amino acid residues that reduces an effector function of the immunoglobulin heavy chain constant region and reduces binding of the antibody to the neonatal Fc receptor (FcRn). In certain embodiments, the immunoglobulin heavy chain constant region comprises an alteration to one or more amino acid residues that reduces an effector function of the immunoglobulin heavy chain constant region. In certain embodiments, the immunoglobulin heavy chain constant region comprises an alteration to one or more amino acid residues that reduces binding of the antibody to the neonatal Fc receptor (FcRn).

In some embodiments, the Fc domain comprises alterations to a heavy chain constant region that reduces effector function associated with a heavy chain constant region, such as, the ability to fix complement, promote phagocytosis, or recruit other immune effector cells (e.g., NK cells) to the heavy chain constant region. In certain embodiments, the alteration to one or more amino acid residues that reduces the effector function of the immunoglobulin heavy chain constant region is an alteration that reduces complement dependent cytotoxicity (CDC), antibody-dependent cell-cytotoxicity (ADCC), antibody-dependent cell-phagocytosis ADCP, or a combination thereof. In certain embodiments, the alteration to one or more amino acid residues that reduces the effector function of the immunoglobulin heavy chain constant region is selected from the list consisting of: (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) K322A, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) E233P, (ooo) L234A, L235E, G237A, A330S, and P331S or (ppp) any combination of (a)-(ooo), per EU numbering. In certain embodiments, the alteration to one or more amino acid residues that reduces the effector function of the immunoglobulin heavy chain constant region comprises L234A, L235E, G237A, A330S, and P331S per EU numbering.

In some embodiments, the Fc domain comprises alterations to a heavy chain constant region that reduces the serum half-life of the antibody. In certain embodiments, the amino acid alteration that alters or reduces binding of the antibody to the neonatal Fc receptor (FcRn) reduces the serum half-life of the antibody. In certain embodiments, the alteration that alters or reduces binding of the antibody to the neonatal Fc receptor (FcRn) is to an amino acid residue selected from the list consisting of: 251, 252, 253, 254, 255, 288, 309, 310, 312, 385, 386, 388, 400, 415, 433, 435, 436, 439, 447, and combinations thereof per EU numbering. In certain embodiments, the alteration that alters or reduces binding of the antibody to the neonatal Fc receptor (FcRn) is to an amino acid residue selected from the list consisting of: 253, 254, 310, 435, 436 and combinations thereof per EU numbering. In certain embodiments, the alteration that alters or reduces binding of the antibody to the neonatal Fc receptor (FcRn) is to an amino acid residue selected from the list consisting of: I253A, I253D, I253P, S254A, H310A, H310D, H310E, H310Q, H435A, H435Q, Y436A, and combinations thereof per EU numbering. In certain embodiments, the alteration that alters or reduces binding of the antibody to the neonatal Fc receptor (FcRn) is to an amino acid residue selected from the list consisting of: I253A, S254A, H310A, H435Q, Y436A and combinations thereof per EU numbering. In certain embodiments, the alteration that alters or reduces binding of the antibody to the neonatal Fc receptor (FcRn) is to an amino acid residue selected from the list consisting of: I253A, H310A, H435Q, and combinations thereof per EU numbering. In certain embodiments, the alteration that alters or reduces binding of the antibody to the neonatal Fc receptor (FcRn) is to an amino acid residue selected from the list consisting of: H310A, H435Q, and combinations thereof per EU numbering.

In certain embodiments, the alteration to one or more amino acid residues that reduces the effector function of the immunoglobulin heavy chain constant region is selected from the list consisting of: (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 31 IS, (ee) 316F, (ff) 328V, (gg) 330R, (hh) 339E or 339L, (ii) 343I or 343V, (jj) 373A, 373G, or 3735, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) K322A, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) E233P, (ooo) L234A, L235E, G237A, A330S, and P331S or (ppp) any combination of (a)-(ppp), per EU numbering.

In certain embodiments, the alteration to one or more amino acid residues that reduces the effector function of the immunoglobulin heavy chain constant region comprises L234A, L235E, G237A, A330S, and P331S per EU numbering. In certain embodiments, the Fc domain comprises an alteration to one or more amino acid residues that alters binding of the antibody to the neonatal Fc receptor (FcRn). In certain embodiments, the alteration to one or more amino acid residues that alters binding of the antibody to the neonatal Fc receptor (FcRn) is to an amino acid residue selected from the list consisting of: I253A, I253D, I253P, S254A, H310A, H310D, H310E, H310Q, H435A, H435Q, Y436A, and combinations thereof per EU numbering. In certain embodiments, the alteration to one or more amino acid residues that alters binding of the antibody to the neonatal Fc receptor (FcRn) is to an amino acid residue selected from the list consisting of: I253A, S254A, H310A, H435Q, Y436A and combinations thereof per EU numbering. In certain embodiments, the alteration to one or more amino acid residues that alters binding of the antibody to the neonatal Fc receptor (FcRn) is to an amino acid residue selected from the list consisting of: I253A, H310A, H435Q, and combinations thereof per EU numbering. In certain embodiments, the alteration to one or more amino acid residues that alters binding of the antibody to the neonatal Fc receptor (FcRn) comprises 1253A per EU numbering. In certain embodiments, the alteration to one or more amino acid residues that alters binding of the antibody to the neonatal Fc receptor (FcRn) comprises H310A per EU numbering. In certain embodiments, the alteration to one or more amino acid residues that alters binding of the antibody to the neonatal Fc receptor (FcRn) comprises H435Q per EU numbering.

In certain embodiments, a heavy chain constant region of the antibody comprises a sequence at least 90%, 95%, 97%, 98%, or 99% identical to the sequence set forth in SEQ ID NO: 11. In certain embodiments, a heavy chain constant region of the antibody comprises a sequence identical to SEQ ID NO: 11. In certain embodiments, a heavy chain constant region of the antibody comprises a sequence at least 90%, 95%, 97%, 98%, or 99% identical to the sequence set forth in SEQ ID NO: 11, wherein the heavy chain constant region comprises an I253A substitution per EU numbering.

In certain embodiments, a heavy chain constant region of the antibody comprises a sequence at least 90%, 95%, 97%, 98%, or 99% identical to the sequence set forth in SEQ ID NO: 12. In certain embodiments, a heavy chain constant region of the antibody comprises a sequence identical to SEQ ID NO: 12. In certain embodiments, a heavy chain constant region of the antibody comprises a sequence at least 90%, 95%, 97%, 98%, or 99% identical to the sequence set forth in SEQ ID NO: 12, wherein the heavy chain constant region comprises an S254A substitution per EU numbering.

In certain embodiments, a heavy chain constant region of the antibody comprises a sequence at least 90%, 95%, 97%, 98%, or 99% identical to the sequence set forth in SEQ ID NO: 13. In certain embodiments, a heavy chain constant region of the antibody comprises a sequence identical to SEQ ID NO: 13. In certain embodiments, a heavy chain constant region of the antibody comprises a sequence at least 90%, 95%, 97%, 98%, or 99% identical to the sequence set forth in SEQ ID NO: 13, wherein the heavy chain constant region comprises an H310A substitution per EU numbering.

In certain embodiments, a heavy chain constant region of the antibody comprises a sequence at least 90%, 95%, 97%, 98%, or 99% identical to the sequence set forth in SEQ ID NO: 14. In certain embodiments, a heavy chain constant region of the antibody comprises a sequence identical to SEQ ID NO: 14. In certain embodiments, a heavy chain constant region of the antibody comprises a sequence at least 90%, 95%, 97%, 98%, or 99% identical to the sequence set forth in SEQ ID NO: 14, wherein the heavy chain constant region comprises an H435Q substitution per EU numbering.

In certain embodiments, a heavy chain constant region of the antibody comprises a sequence at least 90%, 95%, 97%, 98%, or 99% identical to the sequence set forth in SEQ ID NO: 15. In certain embodiments, a heavy chain constant region of the antibody comprises a sequence identical to SEQ ID NO: 15. In certain embodiments, a heavy chain constant region of the antibody comprises a sequence at least 90%, 95%, 97%, 98%, or 99% identical to the sequence set forth in SEQ ID NO: 15, wherein the heavy chain constant region comprises an Y436A substitution per EU numbering.

In certain embodiments, a heavy chain constant region of the antibody comprises a sequence at least 90%, 95%, 97%, 98%, or 99% identical to the sequence set forth in SEQ ID NO: 16. In certain embodiments, a heavy chain constant region of the antibody comprises a sequence identical to SEQ ID NO: 16. In certain embodiments, a heavy chain constant region of the antibody comprises a sequence at least 90%, 95%, 97%, 98%, or 99% identical to the sequence set forth in SEQ ID NO: 16, wherein the heavy chain constant region comprises an H310A/H435Q substitution per EU numbering.

In certain embodiments, a heavy chain constant region of the antibody comprises a sequence at least 90%, 95%, 97%, 98%, or 99% identical to the sequence set forth in SEQ ID NO: 17. In certain embodiments, a heavy chain constant region of the antibody comprises a sequence identical to SEQ ID NO: 17. In certain embodiments, a heavy chain constant region of the antibody comprises a sequence at least 90%, 95%, 97%, 98%, or 99% identical to the sequence set forth in SEQ ID NO: 17, wherein the heavy chain constant region comprises a L234A, L235E, G237A, A330S, and P331S substitution per EU numbering.

In certain embodiments, a heavy chain constant region of the antibody comprises a sequence at least 90%, 95%, 97%, 98%, or 99% identical to the sequence set forth in SEQ ID NO: 18. In certain embodiments, a heavy chain constant region of the antibody comprises a sequence identical to SEQ ID NO: 18, wherein the heavy chain constant region comprises a L234A, L235E, G237A, H310A, A330S, and P331S substitution per EU numbering.

In certain embodiments, a heavy chain constant region of the antibody comprises a sequence at least 90%, 95%, 97%, 98%, or 99% identical to the sequence set forth in SEQ ID NO: 19. In certain embodiments, a heavy chain constant region of the antibody comprises a sequence identical to SEQ ID NO: 19. In certain embodiments, a heavy chain constant region of the antibody comprises a sequence identical to SEQ ID NO: 19, wherein the heavy chain constant region comprises a L234A, L235E, G237A, H435Q, A330S, and P331S substitution per EU numbering.

In certain embodiments, a heavy chain constant region of the antibody comprises a sequence at least 90%, 95%, 97%, 98%, or 99% identical to the sequence set forth in SEQ ID NO: 20. In certain embodiments, a heavy chain constant region of the antibody comprises a sequence identical to SEQ ID NO: 20 per EU numbering.

Alterations that effect FcRn binding can reduce the serum half-life of the antibody, thus allowing the skilled artisan to choose a half-life that is suitable for a particular imaging or therapeutic goal. In certain embodiments, the antibody has a serum half-life of about 12 hours to about 120 hours. In certain embodiments, the antibody has a serum half-life of about 12 hours to about 24 hours, about 12 hours to about 36 hours, about 12 hours to about 48 hours, about 12 hours to about 60 hours, about 12 hours to about 72 hours, about 12 hours to about 84 hours, about 12 hours to about 96 hours, about 12 hours to about 108 hours, about 12 hours to about 120 hours, about 24 hours to about 36 hours, about 24 hours to about 48 hours, about 24 hours to about 60 hours, about 24 hours to about 72 hours, about 24 hours to about 84 hours, about 24 hours to about 96 hours, about 24 hours to about 108 hours, about 24 hours to about 120 hours, about 36 hours to about 48 hours, about 36 hours to about 60 hours, about 36 hours to about 72 hours, about 36 hours to about 84 hours, about 36 hours to about 96 hours, about 36 hours to about 108 hours, about 36 hours to about 120 hours, about 48 hours to about 60 hours, about 48 hours to about 72 hours, about 48 hours to about 84 hours, about 48 hours to about 96 hours, about 48 hours to about 108 hours, about 48 hours to about 120 hours, about 60 hours to about 72 hours, about 60 hours to about 84 hours, about 60 hours to about 96 hours, about 60 hours to about 108 hours, about 60 hours to about 120 hours, about 72 hours to about 84 hours, about 72 hours to about 96 hours, about 72 hours to about 108 hours, about 72 hours to about 120 hours, about 84 hours to about 96 hours, about 84 hours to about 108 hours, about 84 hours to about 120 hours, about 96 hours to about 108 hours, about 96 hours to about 120 hours, or about 108 hours to about 120 hours. In certain embodiments, the antibody has a serum half-life of about 12 hours, about 24 hours, about 36 hours, about 48 hours, about 60 hours, about 72 hours, about 84 hours, about 96 hours, about 108 hours, or about 120 hours. In certain embodiments, the antibody has a serum half-life of at least about 12 hours, about 24 hours, about 36 hours, about 48 hours, about 60 hours, about 72 hours, about 84 hours, about 96 hours, or about 108 hours. In certain embodiments, the antibody has a serum half-life of at most about 24 hours, about 36 hours, about 48 hours, about 60 hours, about 72 hours, about 84 hours, about 96 hours, about 108 hours, or about 120 hours.

In certain embodiments, the antibody has a serum half-life of about 1 day to about 10 days. In certain embodiments, the antibody has a serum half-life of about 1 day to about 2 days, about 1 day to about 3 days, about 1 day to about 4 days, about 1 day to about 5 days, about 1 day to about 6 days, about 1 day to about 7 days, about 1 day to about 8 days, about 1 day to about 9 days, about 1 day to about 10 days, about 2 days to about 3 days, about 2 days to about 4 days, about 2 days to about 5 days, about 2 days to about 6 days, about 2 days to about 7 days, about 2 days to about 8 days, about 2 days to about 9 days, about 2 days to about 10 days, about 3 days to about 4 days, about 3 days to about 5 days, about 3 days to about 6 days, about 3 days to about 7 days, about 3 days to about 8 days, about 3 days to about 9 days, about 3 days to about 10 days, about 4 days to about 5 days, about 4 days to about 6 days, about 4 days to about 7 days, about 4 days to about 8 days, about 4 days to about 9 days, about 4 days to about 10 days, about 5 days to about 6 days, about 5 days to about 7 days, about 5 days to about 8 days, about 5 days to about 9 days, about 5 days to about 10 days, about 6 days to about 7 days, about 6 days to about 8 days, about 6 days to about 9 days, about 6 days to about 10 days, about 7 days to about 8 days, about 7 days to about 9 days, about 7 days to about 10 days, about 8 days to about 9 days, about 8 days to about 10 days, or about 9 days to about 10 days. In certain embodiments, the antibody has a serum half-life of about 1 day, about 2 days, about 3 days, about 4 days, about 5 days, about 6 days, about 7 days, about 8 days, about 9 days, or about 10 days. In certain embodiments, the antibody has a serum half-life of at least about 1 day, about 2 days, about 3 days, about 4 days, about 5 days, about 6 days, about 7 days, about 8 days, or about 9 days. In certain embodiments, the antibody has a serum half-life of at most about 2 days, about 3 days, about 4 days, about 5 days, about 6 days, about 7 days, about 8 days, about 9 days, or about 10 days.

In certain embodiments, the heavy chain constant region has a molecular weight of about 10 kDa to about 25 kDa. In certain embodiments, the heavy chain constant region has a molecular weight of about 10 kDa to about 15 kDa, about 10 kDa to about 20 kDa, about 10 kDa to about 25 kDa, about 15 kDa to about 20 kDa, about 15 kDa to about 25 kDa, or about 20 kDa to about 25 kDa. In certain embodiments, the heavy chain constant region has a molecular weight of about 10 kDa, about 15 kDa, about 20 kDa, or about 25 kDa. In certain embodiments, the heavy chain constant region has a molecular weight of at least about 10 kDa, about 15 kDa, or about 20 kDa. In certain embodiments, the heavy chain constant region has a molecular weight of at most about 15 kDa, about 20 kDa, or about 25 kDa.

### Purification of Antibodies

Forms of antibody of the invention may be recovered from culture medium or from host cell lysates. If membrane-bound, it can be released from the membrane using a suitable detergent solution (e.g., Triton-X 100) or by enzymatic cleavage. Cells employed in expression of antibody of the invention can be disrupted by various physical or chemical means, such as freeze-thaw cycling, sonication, mechanical disruption, or cell lysing agents.

It may be desired to purify antibody of the invention from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the antibody of the invention. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular antibody of the invention produced.

When using recombinant techniques, the antibody can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Carter et al., Bio/Technology 10: 163-7 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being a preferred purification technique. The suitability of protein A as an affinity ligand depends on the species and isotype of any immunoglobulin Fc domain that is present in the antibody. Protein A can be used to purify antibodies that are based on human γ1, γ2 or γ4 heavy chains (Lindmark et al., J. Immunol. Meth. 62: 1-13 (1983)). Protein G is recommended for all mouse isotypes and for human 73 (Guss et al., EMBO J. 5: 15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™resin (J. T. Baker, Phillipsburg, NJ) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, and generally at low salt concentrations (e.g., from about 0-0.25M salt).

### Antibodies (Including Antibody Drug Conjugates (ADCs))

In a further embodiment of the invention, an antibody of the invention according to any of the above embodiments or described herein is conjugated to a heterologous moiety or agent, such as, e.g., as described below and including any additional exogenous material as described herein.

Conjugates of an antibody construct may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate H), active esters (such as disuccinimidyl suberate), aldehydes (such as glutaraldehyde), bis-azido compounds (such as bis (p-azidobenzoyl) hexanediamine), bis-diazonium derivatives (such as bis-(pdiazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an illustrative chelating agent for conjugation of radionucleotide to the antibody (see e.g., WO 1994/11026). The linker may be a “cleavable linker” facilitating release of a cytotoxic drug in the cell. For example, an acid-labile linker, peptidase-sensitive linker, photolabile linker, dimethyl linker or disulfide-containing linker may be used (see e.g., Chari et al., Cancer Res. 52:127-131 (1992); U.S. Pat. No. 5,208,020).

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., obtainable from Pierce Biotechnology, Inc., Rockford, IL., U.S.).

### Pharmaceutical Compositions

Provided herein are compositions comprising an antibody described herein. The invention further provides pharmaceutical compositions and formulations comprising at least one antibody of the present invention and at least one pharmaceutically acceptable excipient or carrier. In some embodiments, a pharmaceutical formulation comprises (1) an antibody of the invention, and (2) a pharmaceutically acceptable carrier.

An antibody is formulated in any suitable form for delivery to a target cell/tissue. Pharmaceutical formulations of an antibody of the present invention are prepared by mixing such antibody having the desired degree of purity with one or more optional pharmaceutically acceptable carriers, diluents, and/or excipients (Remington's Pharmaceutical Sciences 16th edition, Osol, A. Ed. (1980)), in the form of lyophilized formulations or aqueous solutions. Pharmaceutically acceptable carriers, diluents, and excipients are generally nontoxic to recipients at the dosages and concentrations employed, and include, but are not limited to: sterile water, buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Pharmaceutical formulations to be used for in vivo administration are generally sterile. This is readily accomplished by filtration through sterile filtration membranes.

Examples of lyophilized antibody formulations are described in U.S. Pat. No. 6,267,958. Aqueous antibody formulations include those described in U.S. Pat. No. 6,171,586 and WO 2006/044908, the latter formulations including a histidine-acetate buffer.

Pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). In some embodiments, a sHASEGP is combined with one or more additional glycosaminoglycanases such as chondroitinases.

The formulation herein may also contain more than one active ingredient as necessary for the particular indication being treated, preferably those with complementary activities that do not adversely affect each other. Such active ingredients are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. Ed. (1980).

In some embodiments, antibodies may be formulated as immunoliposomes. A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes. Liposomes containing the antibody are prepared by methods known in the art, such as described in Epstein et al., Proc Natl Acad Sci USA 82: 3688 (1985); Hwang et al., Proc Natl Acad Sci USA 77: 4030 (1980); U.S. Pat. Nos. 4,485,045 and 4,544,545; and WO1997/38731 published Oct. 23, 1997. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. A chemotherapeutic agent is optionally contained within the liposome (see Gabizon et al., J. National Cancer Inst. 81: 1484 (1989)). Liposomes with enhanced circulation time are disclosed in U.S. Pat. No. 5,013,556.

Sustained-release preparations may be prepared. Suitable examples of sustained-release preparations include semipermeable matrices of solid hydrophobic polymers containing the antibody, which matrices are in the form of shaped articles, e.g., films, or microcapsules.

### Methods of Use

The antibodies described herein are useful for targeting an antigen present within a kidney. Furthermore, the antibodies and immunoconjugates described herein are useful treating a disease, disorder, or condition (e.g., a tumor or cancer) in the kidney of a patient in need thereof, wherein the method comprises administering an antibody or compositions described herein.

Provided herein are methods of delivering an antibody to a kidney of an individual. In some embodiments, the method comprises administering the antibodies described herein (e.g., an immunoglobulin single-chain domain antibody comprising an Fc domain) to the individual. In some embodiments, the method comprises administering the antibodies described herein comprising the antibody (e.g., an immunoglobulin single-chain domain antibody comprising an Fc domain) to the individual. In some embodiments, the method comprises administering the antibodies described herein comprising the antibody (e.g., an immunoglobulin single-chain domain antibody comprising an Fc domain) to the individual.

Also provided herein are methods of imaging a kidney. In some embodiments, the method comprises administering the antibodies described herein (e.g., an immunoglobulin single-chain domain antibody comprising an Fc domain) to the individual. In some embodiments, the method comprises administering the antibodies described herein comprising the antibody (e.g., an immunoglobulin single-chain domain antibody comprising an Fc domain) to the individual. In some embodiments, the method comprises administering the antibodies described herein comprising the antibody (e.g., an immunoglobulin single-chain domain antibody comprising an Fc domain) to the individual.

Further provided are methods of imaging an antigen within a kidney of an individual. In some embodiments, the method comprises administering the antibodies described herein (e.g., an immunoglobulin single-chain domain antibody comprising an Fc domain) to the individual. In some embodiments, the method comprises administering the antibody (e.g., an immunoglobulin single-chain domain antibody comprising an Fc domain) to the individual. In some embodiments, the method comprises administering the antibodies described herein (e.g., an immunoglobulin single-chain domain antibody comprising an Fc domain) to the individual. In some embodiments, the method comprises administering the antibody (e.g., an immunoglobulin single-chain domain antibody comprising an Fc domain) to the individual. In some embodiments, the method comprises administering an antibody herein comprising the antibody (e.g., an immunoglobulin single-chain domain antibody comprising an Fc domain) to the individual.

Provided are also method of treating a kidney disease or disorder (e.g., characterized by the expression of an antigen within the kidney). In some embodiments, the kidney disease or disorder is BK virus nephritis or nephropathy.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

In some embodiments, an antibody of the invention can be used in a method for binding target antigen in an individual suffering from a disorder associated with increased target antigen expression and/or activity, the method comprising administering to the individual the antibody such that target antigen in the individual is bound. In some embodiments, the target antigen is human target antigen, and the individual is a human individual. An antibody of the invention can be administered to a human for therapeutic purposes. Moreover, an antibody of the invention can be administered to a non-human mammal expressing target antigen with which the antibody cross-reacts (e.g., a primate, pig, rat, or mouse) for veterinary purposes or as an animal model of human disease.

An antibody composition of the invention (and any additional therapeutic agent or adjuvant) can be administered by any suitable means, including parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. In addition, the antibody (e.g., multivalent antibody) is suitably administered by pulse infusion, particularly with declining doses of the antibody (e.g., multivalent antibody). Dosing can be by any suitable route, e.g., by injections, such as intravenous or subcutaneous injections, depending in part on whether the administration is brief or chronic.

Antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibodies of the invention are administered to a human patient, in accordance with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. For some embodiments, intravenous or subcutaneous administration of the antibody of the invention is preferred.

For the prevention or treatment of disease, the dosage and mode of administration will be chosen by the physician according to known criteria. The appropriate dosage of antibody of the invention will depend on the type of disease to be treated, as defined above, the severity and course of the disease, whether the antibody of the invention is administered for preventive or therapeutic purposes, previous therapy, the patient's clinical history and response to the antibody, and the discretion of the attending physician. Preferably, the antibody or composition is administered by intravenous infusion or by subcutaneous injections. Depending on the type and severity of the disease, about 1 g/kg to about 50 mg/kg body weight (e.g., about 0.1-15 mg/kg/dose) of antibody be an initial candidate dosage for administration to the patient, whether, for example, by one or more separate administrations, or by continuous infusion. A dosing regimen can comprise administering an initial loading dose of about 4 mg/kg, followed by a weekly maintenance dose of about 2 mg/kg of the antibody of the invention. However, other dosage regimens may be useful. A typical daily dosage might range from about 1 g/kg to 100 mg/kg or more, depending on the factors mentioned above. For repeated administrations over several days or longer, depending on the condition, the treatment is sustained until a desired suppression of disease symptoms occurs. The progress of this therapy can be readily monitored by conventional methods and assays and based on criteria known to the physician or other persons of skill in the art.

The dose and administration schedule may be selected and adjusted based on the level of disease, or tolerability in the subject, which may be monitored during the course of treatment. The conjugates of the present invention may administered once per day, once per week, multiple times per week, but less than once per day, multiple times per month but less than once per day, multiple times per month but less than once per week, once per month, once per five weeks, once per six weeks, once per seven weeks, once per eight weeks, once per nine weeks, once per ten weeks, or intermittently to relieve or alleviate symptoms of the disease. Administration may continue at any of the disclosed intervals until remission of the tumor or symptoms of the disease being treated. Administration may continue after remission or relief of symptoms is achieved where such remission or relief is prolonged by such continued administration.

For some embodiments, the effective amount of the antibody may be provided as a single dose.

The antibodies of the present invention maybe used in combination with conventional and/or novel methods of treatment or therapy or separately as a monotherapy. In some embodiments, the antibodies of the present invention maybe used with one or more radiation sensitizer agents. Such agents include any agent that can increase the sensitivity of diseases tissue or cells to radiation therapy. In other embodiments, antibodies of the present invention may be used in combination with novel and/or conventional agents that can augment the biological effects of radiotherapy. Irradiation of a tumor can cause a variety of biological consequences which can be exploited by combining antibodies of the present invention with agents that target relevant pathways. In some embodiments, such agents may reduce tumor angiogenesis, or inhibit local invasion and metastasis, or prevent repopulation, or augment the immune response, or deregulate cellular energetics, or reduce population, or alter tumor metabolism, or increase tumor damage, or reduce DNA repair. In certain embodiments, agents for use in combination with antibodies of the present invention may comprise DDR inhibitors, e.g., PARP, ATR, Chk1, or DNA-PK; or survival signaling inhibitors, e.g., mTOR, PI3k, NF-kB; or antihypoxia agents, e.g, HIF-1-alpha, CAP, or UPR; or metabolic inhibitors, e.g., MCT1, MCT4 inhibitors; or immunotherapeutics, e.g., anti-CTLA4, anti-PD-1; or inhibitors of growth factor signal transduction, e.g., EGFR or MAPK inhibitors; or anti-invasives, e.g., kinase inhibitors, chemokine inhibitors, or integrin inhibitors; or anti-angiogenic agents, e.g., VEGF-inhibitors.

Antibodies of the present invention may (i) inhibit the growth or proliferation of a cell to which they bind; (ii) induce the death of a cell to which they bind; (iii) inhibit the delamination of a cell to which they bind; (iv) inhibit the metastasis of a cell to which they bind; or (v) inhibit the vascularization of a tumor comprising a cell to which they bind. In this context, “inhibiting cell growth or proliferation” means decreasing a cell's growth or proliferation by at least 10%, 20%, 30%, 40%, 50%, 60%, 70%, 80%, 90%, 95%, or 100%, and includes inducing cell death.

In certain embodiments, the antibody may be administered from about 0.5 mg/kg to about 30 mg/kg. In certain embodiments, the antibody may be administered from about 0.5 mg/kg to about 1 mg/kg, about 0.5 mg/kg to about 2 mg/kg, about 0.5 mg/kg to about 5 mg/kg, about 0.5 mg/kg to about 10 mg/kg, about 0.5 mg/kg to about 3 mg/kg, about 0.5 mg/kg to about 4 mg/kg, about 0.5 mg/kg to about 5 mg/kg, about 0.5 mg/kg to about 10 mg/kg, about 0.5 mg/kg to about 20 mg/kg, about 0.5 mg/kg to about 30 mg/kg, about 1 mg/kg to about 2 mg/kg, about 1 mg/kg to about 5 mg/kg, about 1 mg/kg to about 10 mg/kg, about 1 mg/kg to about 3 mg/kg, about 1 mg/kg to about 4 mg/kg, about 1 mg/kg to about 5 mg/kg, about 1 mg/kg to about 10 mg/kg, about 1 mg/kg to about 20 mg/kg, about 1 mg/kg to about 30 mg/kg, about 2 mg/kg to about 5 mg/kg, about 2 mg/kg to about 10 mg/kg, about 2 mg/kg to about 3 mg/kg, about 2 mg/kg to about 4 mg/kg, about 2 mg/kg to about 5 mg/kg, about 2 mg/kg to about 10 mg/kg, about 2 mg/kg to about 20 mg/kg, about 2 mg/kg to about 30 mg/kg, about 5 mg/kg to about 10 mg/kg, about 5 mg/kg to about 3 mg/kg, about 5 mg/kg to about 4 mg/kg, about 5 mg/kg to about 5 mg/kg, about 5 mg/kg to about 10 mg/kg, about 5 mg/kg to about 20 mg/kg, about 5 mg/kg to about 30 mg/kg, about 10 mg/kg to about 3 mg/kg, about 10 mg/kg to about 4 mg/kg, about 10 mg/kg to about 5 mg/kg, about 10 mg/kg to about 10 mg/kg, about 10 mg/kg to about 20 mg/kg, about 10 mg/kg to about 30 mg/kg, about 3 mg/kg to about 4 mg/kg, about 3 mg/kg to about 5 mg/kg, about 3 mg/kg to about 10 mg/kg, about 3 mg/kg to about 20 mg/kg, about 3 mg/kg to about 30 mg/kg, about 4 mg/kg to about 5 mg/kg, about 4 mg/kg to about 10 mg/kg, about 4 mg/kg to about 20 mg/kg, about 4 mg/kg to about 30 mg/kg, about 5 mg/kg to about 10 mg/kg, about 5 mg/kg to about 20 mg/kg, about 5 mg/kg to about 30 mg/kg, about 10 mg/kg to about 20 mg/kg, about 10 mg/kg to about 30 mg/kg, or about 20 mg/kg to about 30 mg/kg. In certain embodiments, the antibody may be administered at about 0.5 mg/kg, about 1 mg/kg, about 2 mg/kg, about 5 mg/kg, about 10 mg/kg, about 3 mg/kg, about 4 mg/kg, about 5 mg/kg, about 10 mg/kg, about 20 mg/kg, or about 30 mg/kg. In certain embodiments, the antibody may be administered at least about 0.5 mg/kg, about 1 mg/kg, about 2 mg/kg, about 5 mg/kg, about 10 mg/kg, about 3 mg/kg, about 4 mg/kg, about 5 mg/kg, about 10 mg/kg, or about 20 mg/kg. In certain embodiments, the antibody may be administered at most about 1 mg/kg, about 2 mg/kg, about 5 mg/kg, about 10 mg/kg, about 3 mg/kg, about 4 mg/kg, about 5 mg/kg, about 10 mg/kg, about 20 mg/kg, or about 30 mg/kg.

### EXAMPLES

The following examples are included for illustrative purposes only and are not intended to limit the scope of the present disclosure.

### Example 1. VHH-Fc Preparation

VHH-Fc plasmids were generated by cloning the VHH sequence, with a hinge and Fc portion(human IgG1 CH2-CH3) into a mammalian expression vector. In some instances, mutations were introduced into the Fc portion. To produce recombinant VHH-Fc and variants thereof, plasmid was transfected into HEK293.SUS cells (ATUM, or similar). After 3-5 days of secretion, the antibody-containing supernatant was cleared of cells by centrifugation and sterile filtration. Antibodies were purified using Mab Select SuRe PCC column (GE, Cat #: 11003495) and buffer exchange into PBS, pH 7.0. Proteins were quantified using A280 or BCA. The purity of the antibodies were tested by SDS-PAGE, capillary electrophoresis, HPLC-SEC and LC-MS using standard protocols. Regarding VHH polypeptides, see, for example, McMahon et al., Nature Structural & Molecular Biology|VOL 25|MARCH 2018|289-296 Yeast surface display platform for rapid discovery of conformationally selective nanobodies; Moutel et al., eLife 2016; 5:e16228 NaLi—H1: A universal synthetic library of humanized nanobodies providing highly functional antibodies and intrabodies. De Genst E, Saerens D, Muyldermans S, Conrath K. Antibody repertoire development in camelids. Dev Comp Immunol. 2006; 30(1-2):187-98. doi: 10.1016/j.dci.2005.06.010. PMID: 16051357. Vincke C, Gutierrez C, Wernery U, Devoogdt N, Hassanzadeh-Ghassabeh G, Muyldermans S. Generation of single domain antibody fragments derived from camelids and generation of manifold constructs. Methods Mol Biol. 2012; 907:145-76. doi: 10.1007/978-1-61779-974-7_8. PMID: 22907350. Arbabi Ghahroudi M, Desmyter A, Wyns L, Hamers R, Muyldermans S. Selection and identification of single domain antibody fragments from camel heavy-chain antibodies. FEBS Lett. 1997 Sep. 15; 414(3):521-6. doi: 10.1016/s0014-5793(97)01062-4. PMID: 9323027.

For VHH humanization, see, for example, Vincke C, Loris R, Saerens D, Martinez-Rodriguez S, Muyldermans S, Conrath K. General strategy to humanize a camelid single-domain antibody and identification of a universal humanized nanobody scaffold. J Biol Chem. 2009 Jan. 30; 284(5):3273-84. doi: 10.1074/jbc.M806889200. Epub 2008 Nov. 14. PMID: 19010777.

For VHH stability, see, for example, Kunz P, Flock T, Soler N, Zaiss M, Vincke C, Sterckx Y, Kastelic D, Muyldermans S, Hoheisel J D. Exploiting sequence and stability information for directing nanobody stability engineering. Biochim Biophys Acta Gen Subj. 2017 September; 1861(9):2196-2205. doi: 10.1016/j.bbagen.2017.06.014. Epub 2017 Jun. 20. PMID: 28642127; PMCID: PMC5548252; Kunz P, Zinner K, Mucke N, Bartoschik T, Muyldermans S, Hoheisel J D. The structural basis of nanobody unfolding reversibility and thermoresistance. Sci Rep. 2018 May 21; 8(1):7934. doi: 10.1038/s41598-018-26338-z. PMID: 29784954; PMCID: PMC5962586.

A number of VHH-Fc prototypes and variants were engineered using VHH sequences such as the anti-HER2 clone 2RS15d VHH (See. e.g., WO2016/016021) (SEQ ID NO: 20), and the anti-DLL3 clone hz10D9v7.251 VHH sequences (See e.g., W02020/07967) (SEQ ID NO: 30), unless otherwise stated herein the data collected and shown was obtained using VHH antigen binding regions of these clones.

TABLE 1
Constructs

| | | Fc | |
|---|---|---|---|
| VHH Fc | | Effector | |
| name | FcRn Mutant | Mutant | Target |
| H101 | wt | wt | HER2 |
| D102 | wt | wt | DLL3 |
| H105 | I253A | wt | HER2 |
| H106 | S254A | wt | HER2 |
| H107 | H310A | wt | HER2 |
| H108 | H435Q | wt | HER2 |
| H109 | Y463A | wt | HER2 |
| D111 | I253A | wt | DLL3 |
| D112 | S254A | wt | DLL3 |
| D113 | H310A | wt | DLL3 |
| D114 | H435Q | wt | DLL3 |
| D115 | Y463A | wt | DLL3 |
| H133 | wt | AEASS | HER2 |
| D134 | wt | AEASS | DLL3 |
| H135 | H310A | AEASS | HER2 |
| D136 | H310A | AEASS | DLL3 |
| H137 | H435Q | AEASS | HER2 |
| D138 | H435Q | AEASS | DLL3 |

Variants per EU numbering; AEASS = L234A, L235E, G237A, A330S, and P331S

### Example 2. Antibody Binding Properties: Assays for Target Protein and Target Cells

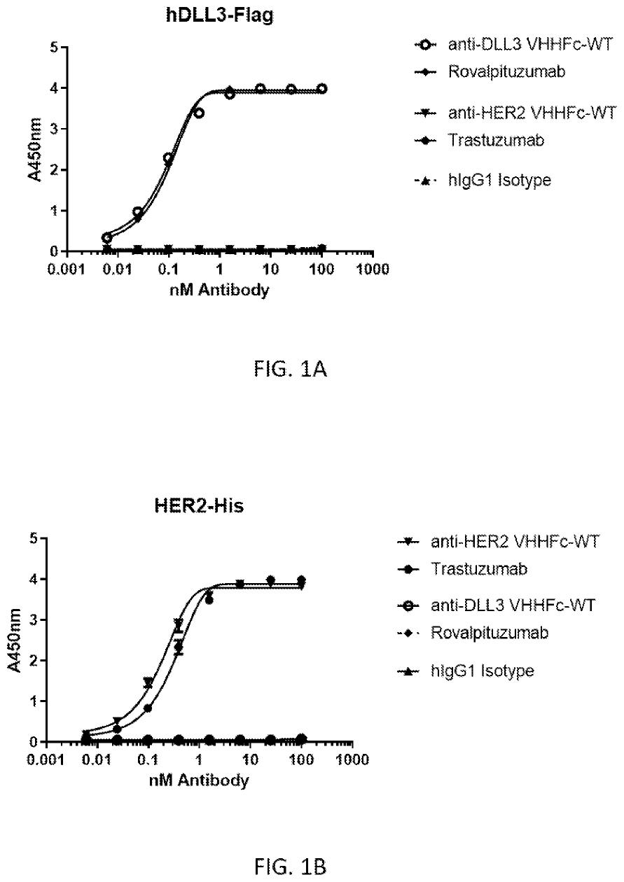

The VHH-Fcs were assessed by ELISA for binding to Target soluble protein—human, murine and cynomolgous orthologs as appropriate, according to standard protocols. Antigens were sourced commercially or produced by cloning known antigen sequences (Uniprot) into mammalian expression vectors with a HIS, FLAG or equivalent tag for purification and detection purposes. A commercially available control anti-target IgG was included. Plates (96-well maxisorp, Corning 3368) were coated with 50 to 100 L of each Target protein of interest at a concentration optimized for coating. Purified VHH-Fc andhIgG1 isotype control (Sigma, Cat #I5154) were prepared at starting concentrations of 200 to400 nM and titrated 1:4 down. Following primary antibody incubation for 1 hour at room temperature (RT), and washing, 0.2 ug/ml of secondary HRP-labelled antibody was added and incubated for 1 h at RT (goat anti human-IgG-Fc-HRP Jackson, Cat #109-035-098). Reaction was detected using 50 L/well of TMB (Neogen, Cat #308177). The color development was stopped with 1 M HCl (50 l). Optical density (OD) was measured at 450 nm using Spectromax plate reader and data were processed using SoftMaxPro. Data shows anti-Target VHH-Fcs bind to human, murine and cynomolgous target protein. Recombinant DLL3 protein used was human DLL3.FLAG(Adipogen #AG-40B-0151, amino acid 27-466), or human DLL3.HIS (abcam #ab255797, amino acid 27-492), or murine DLL3.HIS (IPA custom, amino acid 25-477) or cynomolgous DLL3.HIS (Acrobiosystems #, amino acid 27-490). Control antibodies for DLL3 binding was Rovalpituzumab (Creative Biolabs #TAB-216CL) Recombinant HER2 protein used was human Her2.HIS (Sinobiologics, #10004-H08H) and murine HER2.HIS (Sinobiologics #50714-M08H). Control antibody for HER2 binding was Trastuzumab (DIN: 02240692, ROCHE).). FIGS. 1A and 1B show Anti-Her2 and anti-DLL3 VHH-Fcs binding specifically to soluble target antigen in an ELISA, additional VHH-Fcs comprising mutations in the Fc region to decrease effector function and/or FcRn binding were tested but did not significantly affect binding to target antigen.

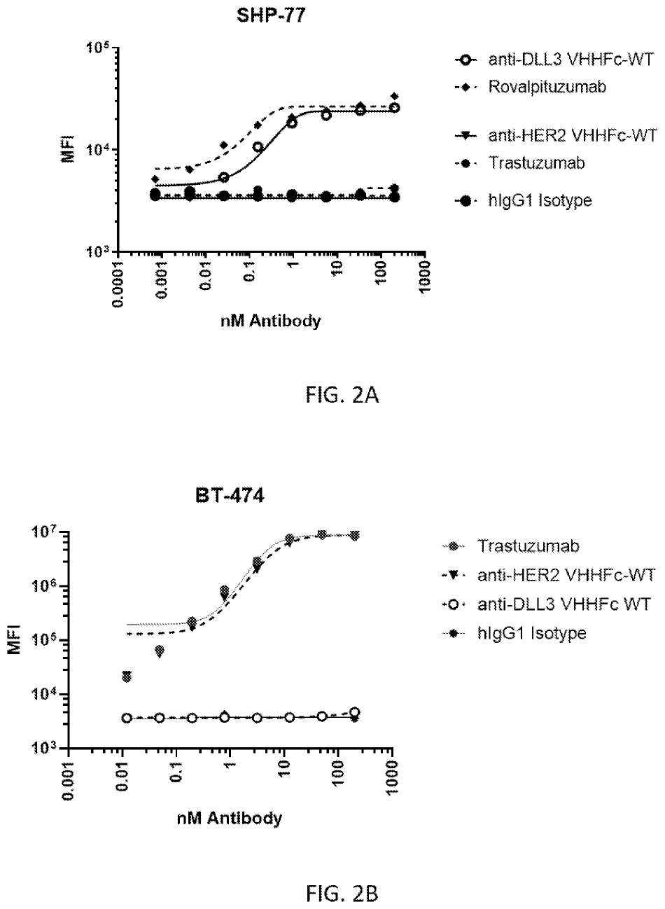

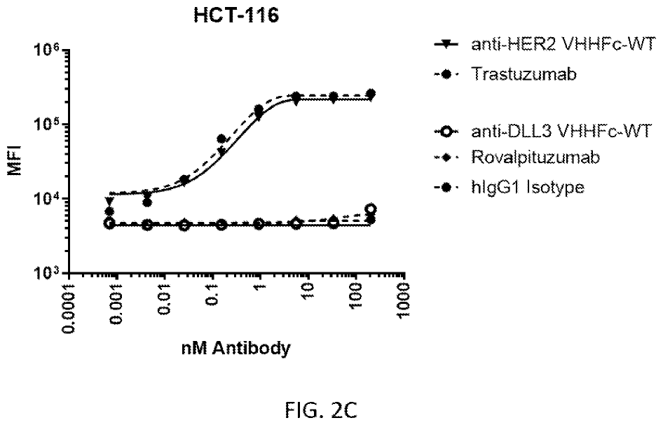

VHH-Fcs were screened for binding to a range of target-positive cancer cell lines by flow cytometry. All cell lines were sourced from ATCC unless otherwise noted, and cultured according to manufacturer's instructions and recommended media. HER2-positive cell lines used were SKBR3(ATCC #HTB-30) and BT474(ATCC #HTB-20) and HEK293-6E(NRC) cells. DLL3-positive cell lines tested include SHP-77(ATCC CR1-2195), NCI-H82(ATCC HTB-175), NCI-H69(ATCC HTB-119), HEK-DLL3 (Creative Biogene #CSC-R00531). HER2-negative cell lines tested included SHP-77. DLL3-negative cell lines tested included HCT-116 (CCL-247), BT-474 and SKBR3. Primary antibodies diluted in same manner as for ELISA were added to cells and incubated for 1 hour on ice. Cells were washed twice with 1% FBS in PBS, centrifuged at 450G for 4 minutes and incubated with 2 μg/mL AlexaFluor 647 conjugated anti-human IgG (Jackson, Cat #109-605-098) or AlexaFluor 647 conjugated anti-mouse IgG (Jackson, Cat #115-605-164) with 1:1000 DAPI (Biolegend, Cat #422801) for 30 minutes on ice. Following two further washes, cells were resuspended, and analyzed by flow cytometry on the iQue screener platform (Intellicyt), and data was processed with Forecyt, according to standard protocols. FIGS. 2A, 2B and 2C show binding to target-positive cell lines and shows that binding was specific to Target-positive cells (i.e., through binding comparison to negative controls cells). Further experiments indicated that Fc mutations to reduce effector function and/or FcRn binding did not impact binding to cancer cells as compared to wildtype Fcs.

### Example 3. Internalization Assays

VHH-Fcs were tested for internalization by target-expressing cells using a secondary antibody conjugated to a pH sensitive dye. Goat anti-hu IgG-Fc secondary antibody was amine-conjugated to a pH sensitive pHAb dye (Promega Cat #G9845) according to the manufacturer's instructions. The pHAb dye has low or no fluorescence at pH >7 but fluoresces in acidic environment upon antibody internalization. Target-positive cells and target-negative cells were plated at 1.0×106/mL in a 96-well V bottom plate.

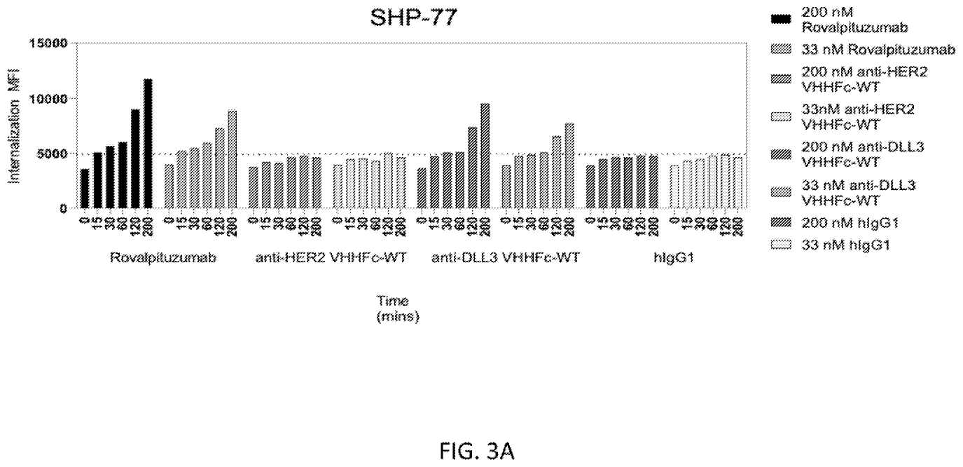

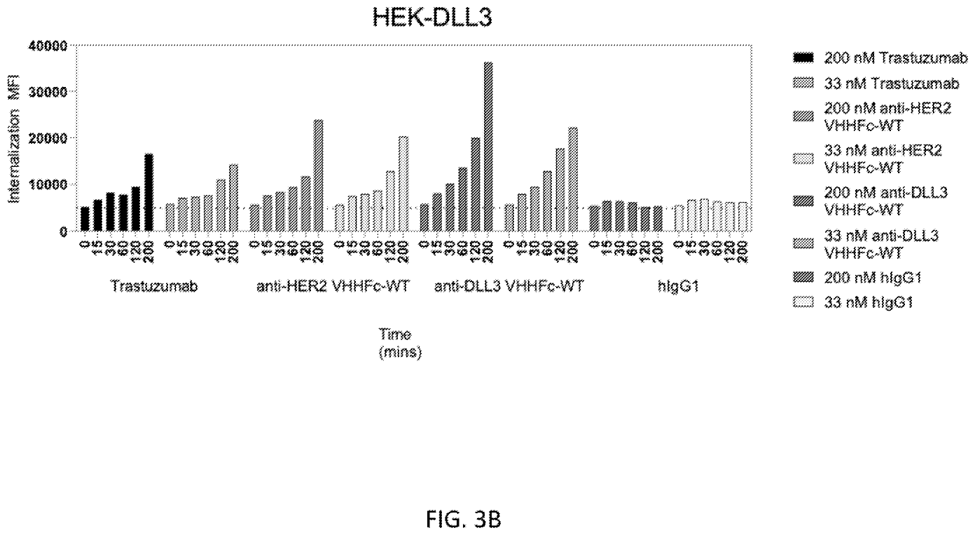

VHH-Fcs and hIgG1 isotype control were diluted in media to 75 nM. Cells were spun to remove supernatant, resuspended with the prepared primary antibodies and incubated on ice for 1 hour. Excess primary antibody was washed off from cells and then incubated with pHAb labelled secondary antibody on ice for 30 minutes. Excess secondary was then washed off and cells were resuspended in media. One set of samples was placed in an incubator at 37° C. to allow internalization, and another set was left on ice (0° C.) as a binding only control. Cells were sampled at different time points ranging from 0 to 24 hours. Cells were stained with DAPI and read by flow cytometry on 572/28 channel with iQue screener platform. The VHH-Fcs show higher fluorescence than the negative controls (isotype, buffer) on target-positive cells. FIGS. 3A and 3B show that H101 and were D102 internalized by SHP-77 and HEK-DLL3 cells.

### Example 4. Antibody Thermal Stability Determination

Denaturing temperatures (Tm) of VHH-Fcs were determined from differential scanning fluorimetry (DSF) using Protein Thermo Shift Dye Kit™ (ThermoFisher, Cat #: 4461146). Briefly, A total of 1 g of antibody was used in each reaction. Melting curves of the antibodies were generated using an Applied Biosystems QuantStudio 7 Flex Real-Time PCR System with the recommended settings stated in the kit manual. The Tm's of the antibodies in Table 1 were then determined by using the ThermoFisher Protein Thermal Shift software (v.1.3). Tml of the VHH-Fcs was determined by DSF. Both H101 and D102 showed good thermostability of 67.5±0.1 Celsius. Additional, VHH-Fcs comprising mutations in the Fc region to decrease effector function and/or FcRn binding were tested for thermostability and resulted in slightly lower thermostability (1 to 2 degrees Celsius), but were still within acceptable ranges.

### Example 5. Receptor Density Determination

In order to test efficacy of the immunoconjugate binding with respect to target density receptor density was measured on target positive cell lines. Target density was measured using the ABC (Antibody Binding Capacity) assay. Cancer cells expressing the target of interest, as well as a negative control cell line, were harvested with cell dissociation buffer, seeded at about 5×104 cells per well into 96-well V bottom plate (Sarstedt 82.1583.001). Cells were tested for receptor expression using QuantiBRITE PE beads (BD Cat #340495) and a PE-conjugated anti-hu IgG (Biolegend clone HP6017) following the manufacturers' instructions. In brief, VHH-Fc and isotype control antibodies were prepared at suitable saturating concentrations based on previous experiments. Antibody sample dilutions were incubated with the panel of cell lines on ice for 1 hour. Cells were washed twice with 1% FBS in 1×PBS (FACS buffer), centrifuged at 400 G for 4 min. Cells were then incubated with 4 μg/mL mouse PE-conjugated anti-hu and DAPI (1:1000) for 30 minutes on ice. Cells were washed twice with FACS buffer, centrifuged at 400 G for 4 minutes and resuspended in FACS buffer. Fluorescence intensity on the PE channel was measured on the iQue Screener platform, and data were processed with ForeCyt software. The amount of PE signal generated from the different primary antibody was then fit to a standard curve based off of known PE molecules/Quantibrite bead samples to determine the number of antibody-binding sites per cell. Relative antibody binding sites correlate to the number of antigens or receptors on cell surface. Table 2 shows receptor density numbers for anti-DLL3 and anti-HER2 VHH-Fcs binding to a panel of cancer cell lines and were similar ranges to those reported in literature.

TABLE 2
Estimated number of epitopes/cell for each binder and cell line

| | | SHP- | HEK- | | | HEK293- | HCT- |
|---|---|---|---|---|---|---|---|
| | | 77 | DLL3 | BT474 | H82 | 6E | 116 |
| Anti-DLL3 | Rova | 969 | 1679 | — | 936 | — | — |
| | D102 | 807 | 1734 | — | 794 | — | — |
| Anti-HER2 | Tmab | 625 | 1575 | 356690 | — | 1969 | 2790 |
| | H101 | 572 | 1490 | 401604 | — | 1935 | 2604 |

### Example 6. Affinity of Antibodies to Target Protein

Antibody affinity was assessed using Octet Red96e (ForteBio). The association rate constant (ka), dissociation rate constant (kd) and affinity constant (KD) were measured by biolayer interferometry with anti-hIgG Fc (AHC) capture biosensors (Fortebio cat #18-5063). Each cycle was performed with orbital shake speed of 1,000 rpm. Antigen was titrated 1:2 from a suitable starting concentration in kinetics buffer (Fortebio, Cat #18-1105). A set of AHC biosensors was dipped in kinetics buffer for baseline step of 60 s. Anti-Target VHH-Fc (5 μg/mL, in kinetics buffer) was loaded onto the biosensors for 240 s followed by a second baseline step of 30 s. The IgG captured sensors were dipped into buffer for single reference subtraction to compensate natural dissociation of capture IgG. Each biosensor was then dipped into corresponding concentration of target protein (human, murine or cynomolgus monomeric protein) for 600 s, followed by 1800 s of dissociation time in kinetics buffer, or conditions as optimized. A new set of AHC biosensors was used for every VHH-Fc. The data was analysed by global fit 1:1 model for the association and dissociation step, (Octet software version v11.0). Table 3 shows binding affinity data.

TABLE 3
Affinity of H101 and D102 to target proteins

| VHH-Fc | Analyte | KD (nM) |
|---|---|---|
| D102 | Human DLL3-Flag | 0.472 |
| D102 | Mouse DLL3-His | 8.75 |
| H101 | Human HER2-His | 3.79 |

### Example 7. FcRn and Fc Effector Mutation Affinity Determination

FcRn affinity of VHH-Fc can generally be used to predict the half-life of antibody serum clearance. (See, e.g., Datta-Mannan A et al. “FcRn affinity-pharmacokinetic relationship of five human IgG4 antibodies engineered for improved in vitro FcRn binding properties in cynomolgus monkeys.” Drug Metab Dispos. 2012 August; 40(8):1545-55). Briefly, 10 nM of biotinylated hFcRn (Sino Biological, Cat #: CT071-H27H-B) was captured with the SA biosensor using Octet RED96e (Fortebio). The hFcRN coated biosensor was dipped into the sample solutions in sodium phosphate buffer (100 mM Na2HPO4,150 mM NaCl w/0.05% Tween-20, pH 6.0) with serial concentrations of tested antibodies and the association measured. The dissociation was measured by dipping the biosensors into sodium phosphate buffer without antibody. The KD values were determined using Octet Data Analysis HT 11.0 software. 2:1 (Heterogeneous Ligand) binding model was used in analysis. Table 4 shows FCRN affinity for wildtype VHH-Fcs, and the impact of specific mutations in the Fc on affinity for the mutants. Changes in FcRn affinity were consistent across targets. Constructs with Fc Effector mutation only have no impact on FcRn affinity. Addition of Fc Effector mutations to FcRn mutation constructs does not affect FcRn affinity. Table 4a shows affinities of VHH-Fcs and Fc variants to FcRn.

TABLE 4a
Affinity of FcRn VHH-Fcs and Fc variants to FcRn

| VHH.Fc | FcRn Mutant | KD (nM) |
|---|---|---|
| H101 | wt | 3.7 |
| D102 | wt | 3.8 |
| H105 | I253A | Weak |
| H106 | S254A | 13 |
| H107 | H310A | No binding |
| H108 | H435Q | Weak |
| H109 | Y463A | 13 |
| H110 | H310A/H435Q | No binding |
| D111 | I253A | Weak |
| D112 | S254A | 19 |
| D113 | H310A | No binding |
| D114 | H435Q | Weak |
| D115 | Y463A | 20 |
| D116 | H310A/H435Q | No binding |
| H133 | wt | 2.1 |
| D134 | wt | 1.9 |
| H135 | H310A | No binding |
| D136 | H310A | No binding |
| H137 | H435Q | Weak |
| D138 | H435Q | Weak |

VHH-Fcs were also tested for affinity to FcγRs by biolayer interferometry using the Octet Red96e platform. Each cycle is performed with orbital shake speed of 1,000 rpm. Streptavidin (SA) biosensors (Sartorius 18-5019) were rehydrated for 10 mins using kinetics buffer (PBS+0.1% BSA+0.02% Tween-20). Biotinylated-FcγRs (Acro Biosystems) were then loaded for 40-100 s onto SA biosensors at concentrations ranging between 1-5 μg/mL diluted in PBS. VHH-Fcs were serially diluted 1:2 in sample buffer (PBS+0.02% Tween-20) with starting concentrations ranging between 5000 nM to 37.5 nM. Loaded biosensors were then associated with VHH-Fcs for 60-120 s. VHH-Fc dissociation was measured for 30-900 s in sample buffer. Bound VHH-Fcs were then removed using 3 cycles of 5 s regeneration buffer (150 mM NaCl, 300 mM Sodium Citrate) and 5 s sample buffer. The data was analyzed either using a globally-fitted 1:1 Langmuir binding model (FcγRI) or steady state analysis (Octet software version HT v11.1).

Analysis shows reduction in binding (represented by a higher KD) to FcγRs for constructs with those mutations incorporated as shown in Table 4b.

TABLE 4b
Affinity of FcRn VHH-Fcs and Fc variants to Fc receptors

| | | | FcγRIIa | FcγRIIa | | FcγRIIIa | FcγRIIIa |
|---|---|---|---|---|---|---|---|
| | Fc | FcγRI | (H167) | (R167) | FcγRIIb/c | (F176) | (V176) |
| | mutation | nM KD | nM KD | nM KD | nM KD | nM KD | nM KD |
| Trastuzumab | wt | 0.92 | 270 | 520 | 3700 | 630 | 110 |
| H101 | wt | 1.01 | 340 | 160 | 450 | 1600 | 480 |
| H133 | AEASS | — | — | 2300 | weak | — | — |
| H135 | AEASS + | — | — | 1200 | weak | — | — |
| | H310A | | | | | | |
| H137 | AEASS + | — | — | 1200 | weak | — | — |
| | H435Q | | | | | | |
| D102 | wt | 1.27 | 390 | 530 | 430 | 1200 | 730 |
| D134 | AEASS | — | weak | 460 | 1100 | — | — |
| D136 | AEASS + | — | weak | 570 | 2200 | — | — |
| | H310A | | | | | | |
| D138 | AEASS + | | weak | 520 | 770 | — | — |
| | H435Q | | | | | | |

(—) indicates no binding detected

### Example 8. Self-Association Studies Using AC-SINS

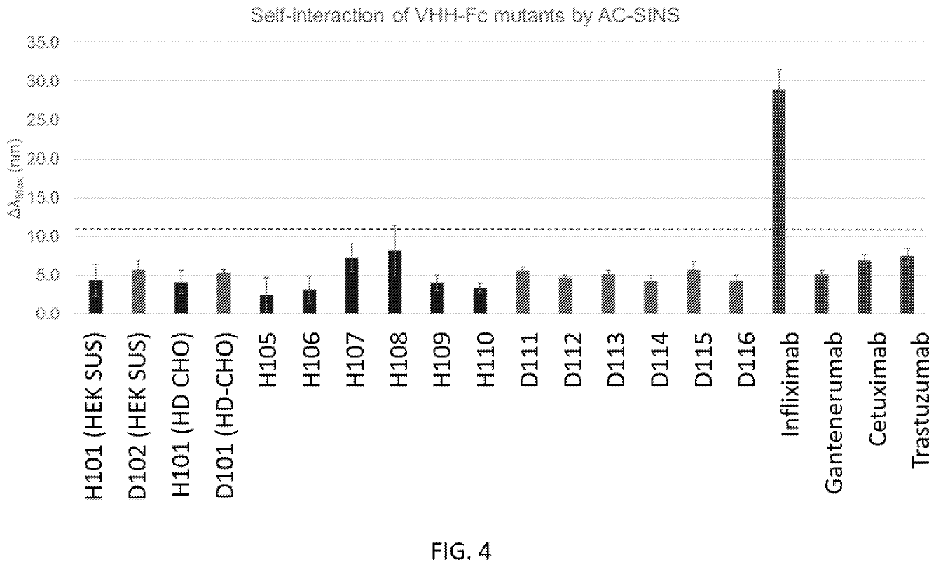

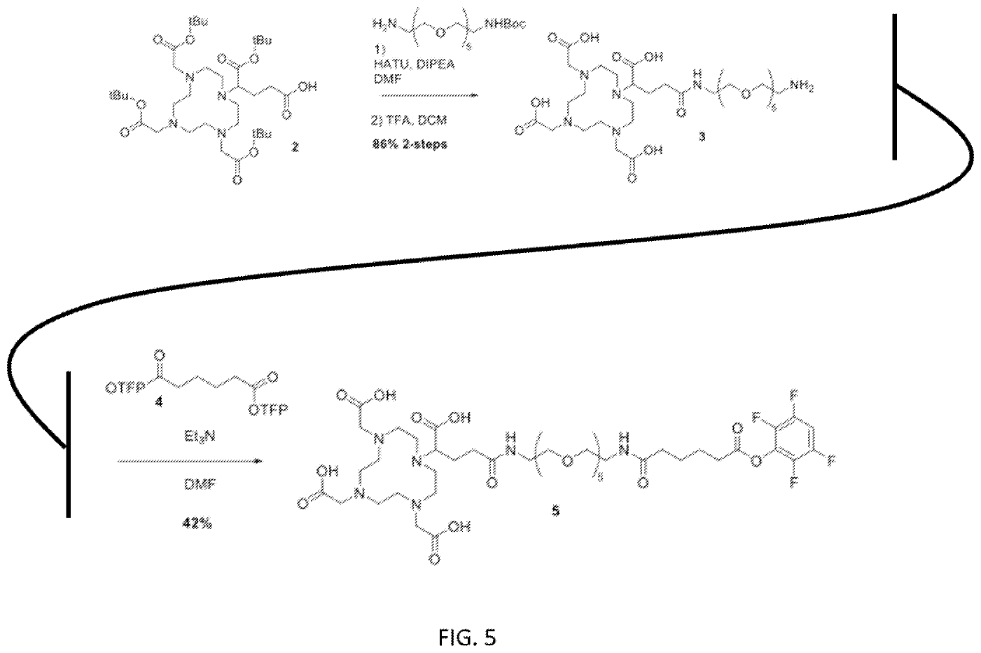

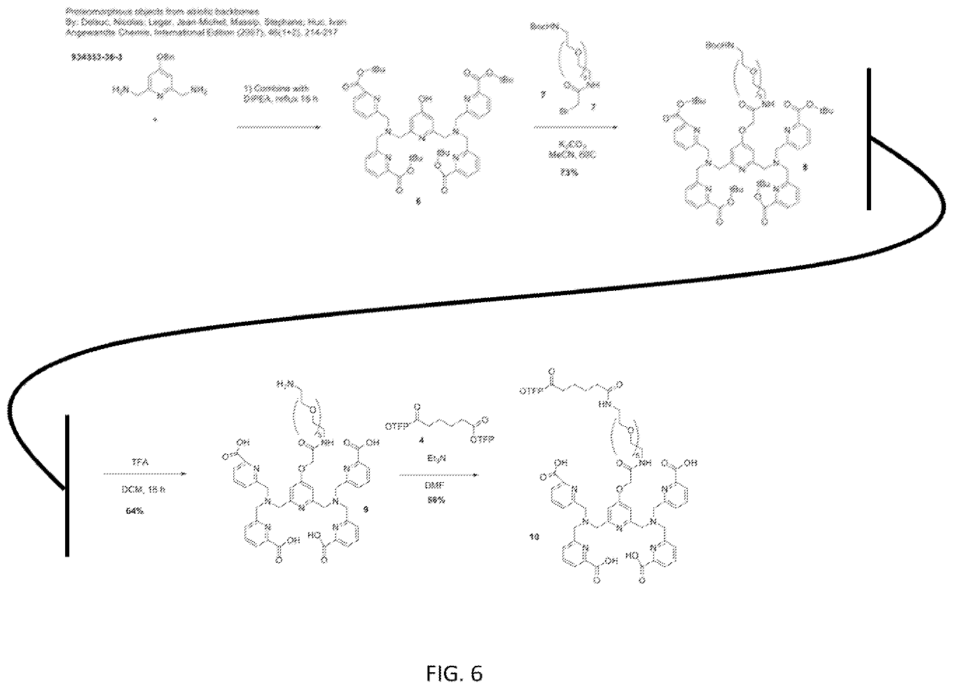

Propensities of self-association of VHH-Fcs was determined from affinity-capture self-interaction nanoparticle spectroscopy (AC-SINS) using gold nanoparticles (Au-NP) (Ted Pella, Cat #: 15705). (PMID: 24492294, 30395473) Briefly, goat IgG and goat anti-human Fc IgG (1:4 mole ratio) were used to coat the Au-NP. Conjugated Au-NP was mixed with 5 μg of each VHH-Fc, in quadruplicates, in a 96-well plate. The wavelength scan was measured with Synergy Neo2 plate reader. The difference of maximum absorbance (Δλmax) was calculated by subtracting λmax of each reaction with that of PBS buffer. The data was analyzed with Linest function in Excel using second-order polynomial fitting. Control antibodies with known high ACSINS score (above the literature established cut-off of 11 for IgGs) were included in the assay. FIG. 4 shows ACSINS scores for test articles and controls.

### Example 9. Polyreactivity Studies

Polyreactivity of VHH-Fcs against negatively charged biomolecules was determined by ELISA (As in Avery et al., “Establishing in vitro in vivo correlations to screen monoclonal antibodies for physicochemical properties related to favorable human pharmacokinetics.” MAbs. 2018 February/March; 10(2):244-255). Briefly, ELISA plate was coated with 5 μg/mL of human insulin (SigmaAlrich, Cat #: 19278) and 10 μg/mL of double stranded DNA (SigmaAlrich, Cat #: D1626-250MG) overnight. The plate was blocked with ELISA buffer (PBS, 1 mM EDTA, 0.05% Tween-20, pH 7.4). 10 μg/mL of test VHH-Fcs was loaded onto the plates in quadruplicates and incubated for 2 hours. Goat anti-human Fc(0.01 ug/ml) conjugated with HRP was then added and the plate incubated for 1 hour. The signal was developed with TMB and A450 absorbance was measured with Synergy Neo2 plate reader. The signal was normalized with the signal of non-coated well for each antibody tested. Table 5 shows the polyreactivity score, in comparison to control antibodies.

TABLE 5
Polyreactivity Assay Scores

| VHH.Fc | Insulin | dsDNA |
|---|---|---|
| H101 | 1.176 | 1.406 |
| D102 | 2.311 | 2.248 |
| H105 | 1.207 | |
| H106 | 1.321 | 1.446 |
| H107 | 1.306 | 1.678 |
| H108 | 1.420 | 1.663 |
| H109 | 1.244 | 1.579 |
| H110 | 1.181 | 1.317 |
| D111 | 2.202 | |
| D112 | 3.461 | 2.970 |
| D113 | 2.829 | 2.594 |
| D114 | 3.161 | 3.015 |
| D115 | 2.503 | 2.252 |
| D116 | 2.446 | 2.302 |
| Gantenerumab | >10 | >10 |

### Example 10. Fc Variants Effectively Reduce VHH-Fc Half-Life

In certain instances, reducing the drug half-life of alpha emitters is important for safety and to avoid unwanted toxicity associated with treatment. However, antibodies generally have a half-life upwards of 14 days or greater. Therefore, the half-life of the VHH-Fc variants was tested in order to observe and measure any reductions in half-life.

Twenty eight (28) 8 week old male B6.Cg-Fcgrttm1Dcr Tg(FCGRT)32Dcr/DcrJ (Tg32 hom, JAX stock #014565) mice were distributed into 7 groups with 4 mice per group as outlined in the table. Tg32 mice comprise a humanized FcRn and are generally viewed as a surrogate for human pharmacokinetics of antibodies when compared to non-human primates. (See, e.g., Avery L B et al. “Utility of a human FcRn transgenic mouse model in drug discovery for early assessment and prediction of human pharmacokinetics of monoclonal antibodies.” MAbs. 2016 August-September; 8(6):1064-78). On Day 0, body weights were measured and test articles were IV administered to all mice at 3 mg/kg and 5 ml/kg. 25 μL blood samples were collected from each mouse at time intervals. The blood samples were collected into 1 μL K3EDTA, processed to plasma, diluted 1/10 in 50% glycerol in PBS, transferred into specialized 96 well storage plates, and stored at −20° C. All plasma samples were assessed via a hIgG ELISA chosen for its high sensitivity for all seven test articles.

TABLE 6
Pharmacokinetic parameter summary for HER2 VHH-Fc

| | Terminal | Clear- | | AUC | Volume of |
|---|---|---|---|---|---|
| | Half-Life | ance | Cmax | μg-days/ | Distribution |
| | days | mL/days | μg/mL | mL | mL |
| H105 | 1.12 | 152.1 | 63.9 | 841 | 137 |
| sem | 0.03 | 3.4 | 3.6 | 29 | 1 |
| H106 | 7.10 | 19.8 | 53.5 | 2193 | 177 |
| sem | 0.31 | 0.8 | 0.4 | 29 | 3 |
| H107 | 0.41 | 304.4 | 62.4 | 516 | 82 |
| sem | 0.01 | 15.0 | 4.3 | 22 | 3 |
| H108 | 1.57 | 117.5 | 46.6 | 903 | 174 |
| sem | 0.10 | 6.6 | 0.7 | 40 | 6 |
| H109 | 6.92 | 18.2 | 52.2 | 2519 | 152 |
| sem | 0.34 | 0.6 | 0.8 | 28 | 4 |
| H101 | 6.91 | 28.7 | 57.0 | 1946 | 218 |
| sem | 0.77 | 5.2 | 1.6 | 231 | 35 |
| trastuzumab | 14.54 | 5.9 | 59.0 | 4108 | 108 |
| sem | 1.12 | 0.5 | 2.3 | 109 | 2 |

As observed in Table 6, the introduction of mutations within the FcRn was generally able to reduce the half-life of the anti-HER2 VHH-Fc. Interestingly, contrary to published results in the field, not all Fc variants when included in the immunoconjugates tested showed a reduction in half-life consistent with previously published results found in the literature. (See, e.g., Burvenich I J et al., “Cross-species analysis of Fc engineered anti-Lewis-Y human IgG1 variants in human neonatal receptor transgenic mice reveal importance of S254 and Y436 in binding human neonatal Fc receptor.” MAbs. 2016 May-June; 8(4):775-86).

TABLE 7
Pharmacokinetic summary for DLL3 VHH-Fc

| | Terminal Half-Life | Clearance |
|---|---|---|
| | days | mL/days |
| D111 | 10.2 | 10.8 |
| SEM | 4.4 | 10.5 |
| D112 | 14.2 | 7.8 |
| SEM | 3.2 | 1.0 |
| D113 | 1.1 | 254.8 |
| SEM | 0.2 | 27.0 |
| D114 | 2.5 | 46.9 |
| SEM | 0.1 | 3.6 |
| D115 | 11.0 | 6.9 |
| SEM | 13.2 | 1.1 |
| D102 | 13.3 | 10.3 |
| SEM | 3.3 | 3.8 |
| trastuzumab | 18.4 | 3.7 |
| SEM | 5.9 | 0.9 |

As observed in Table 7, the introduction of mutations within the FcRn was generally able to reduce the half-life of the anti-DLL3 VHH-Fc. Similarly to HER2 binding immunoconjugates and contrary to published results, not all Fc variants showed a reduction in half-life consistent with previously published results found in the literature.

### Example 11. VHH-Fc Intact Mass Analysis

Conjugates were deglycosylated prior to analysis with in-house Endo-S enzyme (final concentration of 10 μg/mL) at 37° C. for 1 hour.

For analysis of the intact mass, 8 μL samples were injected on a Waters Acquity UPLC-Q-TOF with a UPLC BEH200 SEC 1.7 μM 4.6×150 mm column. These samples were eluted with a mobile phase of water/ACN (70/30, v/v) with 0.1% TFA and 0.1% FA (formic acid) for 11 min with a flow rate of 0.25 mL/min.

### Example 12. Conjugation of VHH-Fc Proteins with Chelator-Linkers

Conjugations can be carried out using many of the methods available for preparation of IgG radioconjugates and IgG antibody-drug conjugates. For information on the range of applicable methodologies, see PW Howard Antibody-Drug Conjugates (ADCs), Protein Therapeutics, First Edition, chapter 9, pp. 278-279 (2017).

For a typical lysine-based conjugation, a VHH-Fc was buffer-exchanged into 0.1 M NaHCO¬3, pH 8.5-9.5 by either Microsep Advance Centrifugal Device (Pall 10K MWCO, Cat #: MCP010C41) or by Zeba column (ThermoFisher, Cat #: 87768), followed by sterilization with a Costar Spin-X Centrifuge Tube, 0.22 μm (Corning, Cat #: 8160). The buffer-exchanged antibody was quantified by BCA assay. An appropriate molar excess (5-20 eq) of chelator-linker (50 mM in DMSO) was added to the VHH-Fc (2 mg/mL final concentration) and the reaction was incubated at 25° C. either for 2 h or overnight in the Thermomixer. After the reaction was complete, the sample was passed through a Zeba column (ThermoFisher, Cat #: 87770) according to the manufacturer's protocol to remove unused chelator-linker and buffer-exchange into PBS (pH 7.4) (LifeTechnologies, Cat #: 10010-023). This VHH-Fc-chelator conjugate (VFCC) was stored at 4° C. until analysis and purification.

### Example 13. VHH-Fc-Chelator Conjugate (VFCC) Purification with SEC

To remove high molecular weight species (HMWS) and low molecular weight species (LMWS), VHH-Fcs were purified by SEC using an AKTA Pure FPLC system with a Cytiva HiLoad 16/600 Superdex 200 pg column. TBS buffer (50 mM Tris, 150 mM NaCl, OmniTrace Ultra water [VWR, Cat #: CAWX0003-2]), pH 7.6 was used for the SEC buffer. The fractions containing intact VHH-Fcs were pooled together and concentrated using Microsep Advance Centrifugal Device (Pall 10k MWCO, Cat #: MCP010C41). The concentrated sample was transferred to an Ultrafree-MC GV Centrifugal Filter, 0.22 μm 0.5 mL (Millipore, Cat #: UFC30GVOS) and spun at 3,000×g for 3 minutes.

### Example 14. Protein Quantification

VHH-Fc protein content was quantified with a Pierce BCA Protein Assay Kit (Thermo, Cat #: 23225) standardized by Cetuximab (LIST/E: 094822, DIN 02271249, 2 mg/mL).

### Example 15. Chelator to VHH-Fc Ratio (CAR) Analysis

The chelator loading ratio, herein described as CAR, can be analyzed through methods applicable to practitioners of the art of antibody conjugates. For a review of these methods in the context of ADCs, see A Wakankar et al., mAbs 3:161 (2011). The CAR of each conjugate was analyzed by DG-SEC-MS.

Conjugates were analyzed through the deglycosylation and UPLC-Q-TOF procedure described in Example 11. In this case, a distribution of masses is obtained after spectrum deconvolution that allows calculation of the average CAR of the preparation.

Conjugates were analyzed through the deglycosylation and UPLC-Q-TOF procedure described in Example 11. In this case, a distribution of masses is obtained after spectrum deconvolution that allows calculation of the average CAR of the preparation.

### Example 16. Binding of VHH-Fc Conjugates to Cells Expressing Target Protein

In some instances, conjugation can negatively impact binding of the VHH-Fc to the target protein. Binding of VHH-Fc conjugates was therefore tested, similar to as described above. Table 8 shows cell binding data of VHH-Fc chelator conjugates.

TABLE 8
Cell binding data of VHH-Fc chelator conjugates
EC50 (nM)

| | | | HCT- | HEK- | HEK- |
|---|---|---|---|---|---|
| | Antibody | SHP-77 | 116 | DLL3 | 293 |
| Controls | Rovalpituzumab | 0.11 | — | 0.06 | — |
| | Trastuzumab | — | 1.16 | 1.06 | 0.69 |
| | hIgG1 | — | — | — | — |
| Short Linker | H101 (CAR 0) | — | 2.21 | 1.62 | 1.14 |
| DOTA | H101 (CAR 0.6) | — | 1.96 | 1.73 | 1.16 |
| p-SCN-Bn- | H101 (CAR 1.1) | — | 2.46 | 1.39 | 1.42 |
| DOTA | H101 (CAR 2.3) | — | 3.34 | 2.05 | 1.68 |
| | H101 (CAR 2.7) | — | 2.96 | 1.88 | 1.58 |
| | H101 (CAR 4.6) | — | 5.63 | 2.99 | 2.27 |
| | H101 (CAR 8.3) | — | 5.32 | 4.43 | 3.52 |
| | D102 (CAR 0) | 0.53 | >100 | 1.42 | >10 |
| | D102 (CAR 0.9) | 0.41 | — | 0.48 | — |
| | D102 (CAR 4.7) | 0.38 | — | 0.56 | — |
| Long Linker | H101 (CAR 0) | — | 2.21 | 1.62 | 1.14 |
| DOTA | H101 (CAR 2.0) | — | 4.01 | 3.99 | 3.11 |
| TFP-Ad-PEG5- | H101 (CAR 8.9) | — | 40.11 | 28.37 | 28.89 |
| DOTA | D102 (CAR 0) | 0.53 | >100 | 1.42 | >10 |
| | D102 (CAR 2.7) | 0.50 | — | 0.58 | — |
| | D102 (CAR 9.3) | 0.60 | — | 0.91 | — |

H101 = Her2 antigen binding;
D102 = DLL3 antigen binding;
CAR = Chelator to VHH ratio

As observed in Table 8, binding was observed for both long and short DOTA linkers. As also shown in Table 8, binding was also observed across increasing chelator VHH-Fc ratios (CAR).

### Example 17. Percent Intact Analysis

The percent intact immunoconjugate was established by HPLC-SEC. 12 μL of conjugate was added to a glass vial insert in a standard HPLC vial. 10 μL of sample was injected onto an Agilent HPLC-SEC with a Wyatt Technology WTC-050S5 SN:0429 BN WBD129 column column and eluted with 1×PBS (100%) for 40 min at a flow rate of 0.5 mL/min

### Example 18. Endotoxin Level Determination

Endotoxin test was performed using Wako's Limulus Amebocyte Lysate Pyrostar™ ES-F Single Test (Cat #: WPESK-0015) according to manufactural protocol. The QC cutoff was set based on the maximum injection dose projected for each animal in the study while following appropriate animal care and FDA guidelines.

### Example 19. Radiolabeling with In-111

40 μg of each of the 4 test articles was diluted to 100 μL with 0.1 M ammonium acetate buffer in a 500 μL lo-bind Eppendorf tube and 18-25 μL (20-22 MBq) of [111In]InCl3 was added and mixed with a pipette. The reaction mixtures were incubated at 37° C. in an incubator for 1 hour. The tubes were then transferred to a 4° C. fridge.

Incorporation of radionuclides was determined by spotting 0.5 μL of sample at the origin of a 1.5×10 cm iTLC strip. The strip was then placed in a 50 mL Falcon tube containing 2 mL of mobile phase (25 mM EDTA in pH 5 0.1 M sodium acetate buffer) until the solvent had reached the top of the strip. The strip was removed and exposed to a phosphor imaging plate which was then scanned in a Cyclone phosphor imager. Regions of interest were drawn over spots corresponding to the migration of protein-bound and un-bound In-111 and the proportion in each calculated.

Radioconjugates were also analyzed by SEC-HPLC: A volume corresponding to 0.1-0.2 MBq of the sample was pipetted into a 500 μL lo-bind Eppendorf tube and the radioactivity measured in an ionization chamber. The sample was drawn up into a syringe and injected onto the HPLC system. Samples were eluted with PBS. The eluate from the system was collected and the radioactivity measured in order to determine the recovery from the column (corrected for activity remaining in the sample tube and the injection syringe).

TABLE 9
Indium-111 Radiolabeling Efficiency

| | | Labelling efficiency | |
|---|---|---|---|
| | | post-synthesis | |
| Chelator-Linker | Antibody | Attempt 1 | Attempt 2 |
| P-SCN-Bn-DOTA | H101 | 95.9% | 96.5% |
| TFP-Ad-PEG5-DOTAGA | | 97.5% | 97.7% |
| P-SCN-Bn-DOTA | D102 | 97.5% | 97.0% |
| TFP-Ad-PEG5-DOTAGA | | 98.1% | 97.2% |

### Example 20. Radiolabeling with Ac-225

800 μg of each of the 4 test articles was diluted to 200 μL with 0.2 M ammonium acetate buffer pH 6.5 in a 500 μL lo-bind Eppendorf tube and 2 μL (400 kBq) of 225-Actinium chloride was added and mixed with a pipette. The reaction mixtures were incubated at 37° C. in an incubator for 1 hour in the case of the Py4 Pa conjugates and 2 hours for the DOTA conjugates. The tubes were then transferred to a 4° C. fridge.

Incorporation was measured by spotting 0.5 μL of sample at the origin of a 1.5×10 cm iTLC strip and allowing it to dry for a few minutes. The strip was then placed in a 50 mL Falcon tube containing 2 mL of mobile phase (25 mM EDTA in pH 5 0.1 M sodium acetate buffer) until the solvent had reached the top of the strip. The strip was removed and allowed to equilibrate for at least 2 hours, after which it was exposed to a phosphor imaging plate which was then scanned in a Cyclone phosphor imager. Regions of interest were drawn over spots corresponding to the migration of protein-bound and un-bound Ac-225 and the proportion in each calculated.

Alternately, samples could be assayed by HPLC-SEC: HPLC of DOTA conjugates used a BioSEP SEC 5 μm s3000 3007.88 mm column with 20% acetonitrile in PBS elution. HPLC of Py4 Pa conjugates used a Wyatt 050S5 5 μm 500 Å 7.8×300 mm column with 20% acetonitrile in PBS elution).

50 μL of each sample was drawn up into a Hamilton syringe and injected onto the HPLC system. From 10-30 minutes post injection, 30 second fractions of the eluate (0.25 mL) were collected by hand into counting tubes. The fractions were allowed to reach secular equilibrium for 24 hours and then measured in a gamma counter. A 5 μL sample of each preparation was also counted to enable the recovery from the HPLC system to be calculated. Radiochemical purity was determined by determining the area under the peak for 18.5-22.5 mins and 19.5-23.5 mins for DOTA and Py4 Pa conjugates, respectively, as a percentage of total counts. As shown in Table 10 all chelator-linker combinations showed good labeling efficiency.

TABLE 10
Ac-225 Radiolabeling Efficiency

| | | iTLC Labelling efficiency |
|---|---|---|
| Chelator-Linker | Antibody | immediately after preparation |
| p-SCN-Bn-DOTA | H101 | 92.0% |
| TFP-Ad-PEG5-DOTAGA | | 96.3% |
| TFP-Ad-PEG5-Py4Pa | | 93.1% |
| p-SCN-Ph-Et-Py4Pa | | 96.0% |
| p-SCN-Bn-DOTA | D102 | 98.5% |
| TFP-Ad-PEG5-DOTAGA | | 99.5% |
| TFP-Ad-PEG5-Py4Pa | | 98.0% |
| p-SCN-Ph-Et-Py4Pa | | 100% |

### Example 21. Stability of VHH-Fc Radioconjugates

The stability of the radiolabeled immunoconjugates was tested, both for 225Ac and 111In. VHH-Fc chelator-conjugates were radiolabeled (either In-i 11 or Ac-225) as described above. For stability in PBS, 50 μL of each labelled test article was then added to either 200 μL of PBS (with In-ill) or 200 uL PBS/ascorbate (with Ac-225) and stored at 4° C. For stability in serum, 50 μL of each labelled test article was added to 200 μL of mouse serum and incubated at 37° C. Aliquots of were taken at different time points and analyzed for radiochemical purity using iTLC and/or HPLC-SEC as described above. The results of these stability experiments are shown in Table ii and Table 12 below and indicated that the radio conjugates were stable in both PBS and serum.

TABLE 11
Stability of Her2 and DLL3 conjugates labeled with In-111

| Radiochemical | DLL3 (D102) | | HER2 (H101) | |
|---|---|---|---|---|
| purity by | | TFP-Ad- | | TFP-Ad- |
| HPLC | P-SCN-Bn- | PEG5- | P-SCN-Bn- | PEG5- |
| (iTLC) | DOTA | DOTAGA | DOTA | DOTAGA |
| PBS 1 h | 97.5% | 98.1% | 97.5% | 98.4% |
| PBS 24 h | 89.1% | 95.2% | 96.5% | 98.4% |
| Serum 24 h | 94% (94%) | 98% (94%) | 97% | 94% |
| Serum 72 h | 92% (92%) | 96% (94%) | 100% (87%) | 100% (84%) |
| Serum 168 h | 92% (94%) | 95% | 95% (91%) | 92% |

TLC radiochemical incorporation values presented in parentheses.
iTLC incorporation >95% except where shown

TABLE 12
Stability of Her2 and DLL3 conjugates labeled with Ac-225

| | DLL3 (D102) | | | | HER2 (H101) | | | |
|---|---|---|---|---|---|---|---|---|
| | P- | TFP- | TFP- | P- | P- | TFP- | TFP- | P- |
| Radiochemical | SCN- | Ad- | Ad- | SCN- | SCN- | Ad- | Ad- | SCN- |
| purity by | Bn- | PEG5- | PEG5- | Ph-Et- | Bn- | PEG5- | PEG5- | Ph-Et- |
| HPLC (iTLC) | DOTA | DOTAGA | Py4Pa | Py4Pa | DOTA | DOTAGA | Py4Pa | Py4Pa |
| PBS 1 h | 91% | 92% | 83% | 82% | 93% | 93% | 84% | N/D |
| PBS 24 h | 92% | 92% | 83% | 83% | 93% | 91% | 82% | 82% |
| Serum 24 h | 88% | 91% | 78% | 69% | 91% | 90% | 75% | 68% |
| | (94%) | | | | | | | |
| Serum 72 h | 89% | 90% | 73% | 65% | 89% | 85% | 74% | 61% |
| | (90%) | (94%) | | | (87%) | (94%) | | |
| Serum 168 h | 81% | 86% | 71% | 59% | 85% | 80% | 70% | 56% |
| | (91%) | | | | (89%) | | | |

TLC radiochemical incorporation values presented in parentheses.
iTLC incorporation >95% except where shown

### Example 22. Immunoreactivity of VHH-Fc Radioconjugates

The immunoreactive fraction (IRF) was determined though a method described by SK Sharma et al. in Nucl. Med. Biol. 2019, 71, 32-38. Samples were incubated overnight in PBS at 4° C. for analysis and before in vivo experiments, while some samples were incubated in serum at 37° C. for 3 and 7 days as an alternate measure of stability.

### Bead Coating

Dynabeads and antigen (0.15 nmol per 0.125 ug beads) were incubated in B/W buffer (25 uL/0.125 ug beads) at room temperature on a tube rotator for 30 minutes. The Eppendorfs were spun at 100×g for 15 seconds and placed on a magnetic rack for 3 minutes. The supernatant was removed and the beads washed with PBSF. 1 mg of beads was then resuspended in 200 μL of B/W buffer and 2 mg in 400 μL of B/W buffer. Control beads were prepared the same way, except with no antigen added to the tubes.

### Immunoreactive Fraction (IRF) Assay

The appropriate volume of beads (25 uL/0.125 mg beads) generated above was added to microcentrifuge tubes, prewashed with 1 mL PBSF. Radiolabeled VHH-Fc-conjugate (10 ng), block (10 or 50 ug unconjugated antibody; if required), and PBSF were added to each reaction to achieve a final volume of 350 μL. The samples were incubated at room temperature on a rotor for 30 minutes. After this the tubes were centrifuged at 100×g for 15 seconds and placed on a magnetic rack for 3 minutes. The supernatant was collected in a gamma counter tube. The beads were washed twice with 400 μL PBSF and collected in a separate gamma counter tube. The beads were finally resuspended in 500 μL PBSF and transferred to a gamma counter tube. The reaction tube was washed with 500 μL PBSF and this was added to the gamma counter tube containing the beads.

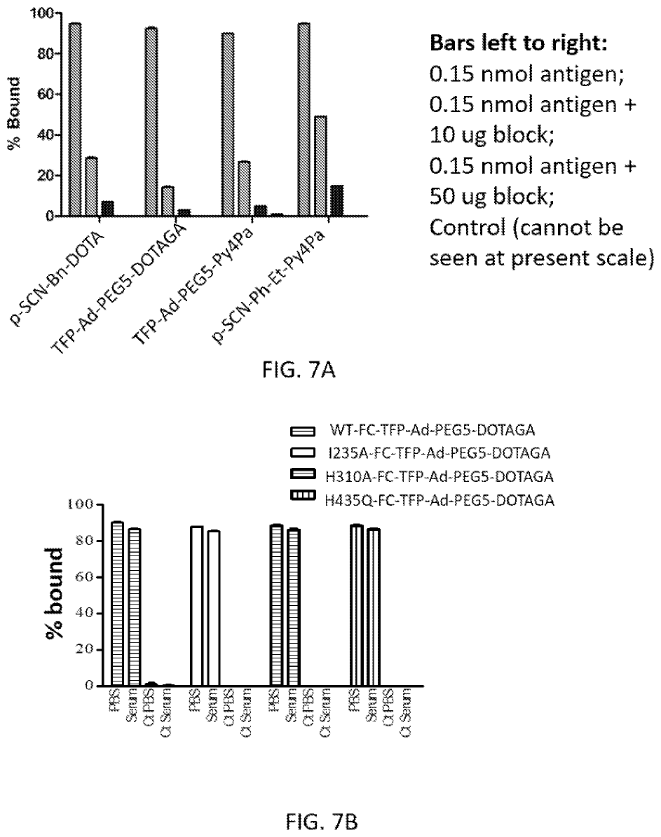

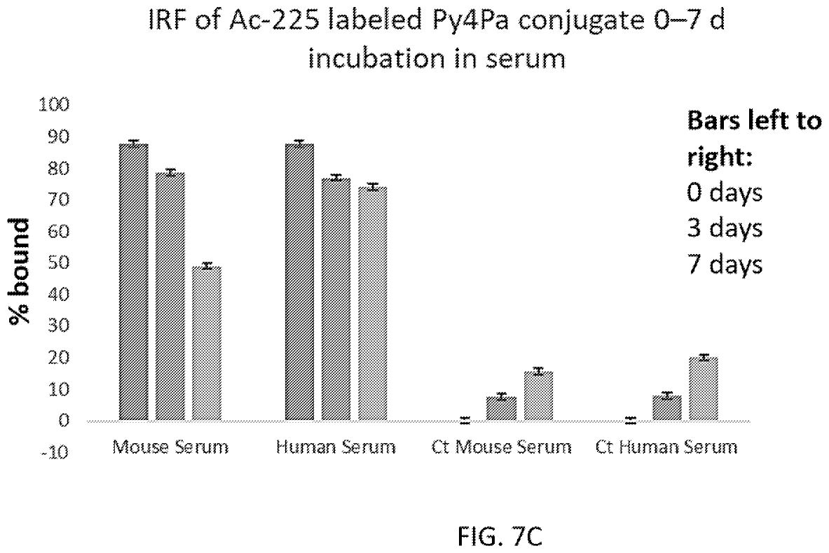

As shown in FIG. 7A for DLL3 all linker chelator combinations showed a similar immunoreactive fraction indicating no bias in labeling based upon the specific linker chelator combination, FIG. 7B shows that there was no effect due to Fc region mutations in immunoreactive fraction after 24 hours in PBS or serum, and FIG. 7C shows the immunoreactive fraction of 225AC labeled anti-DLL3 VHH-Fc (D102) and stability in serum and plasma.

### Example 23. Biodistribution of VHH-Fc Radioimmunoconjugates

Biodistribution and Tissue Accumulation Over Time in HER2+BT474 Tumors

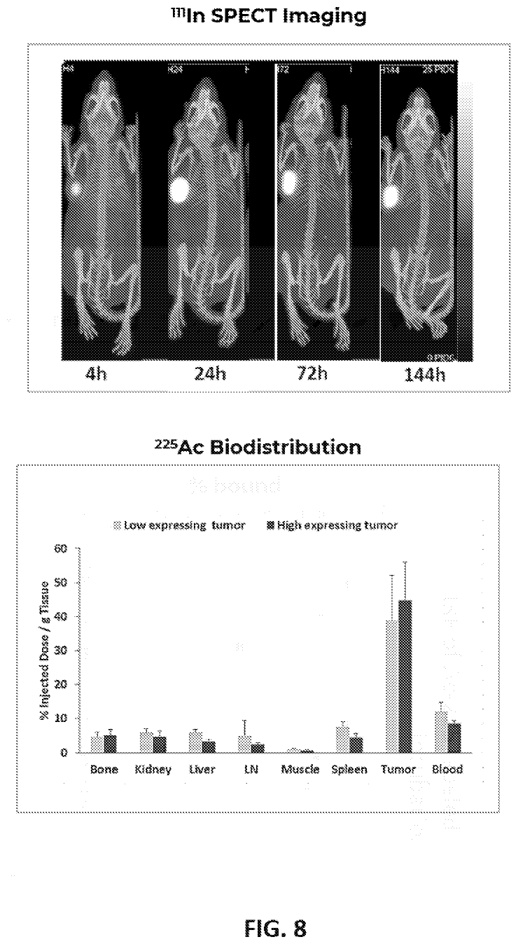

Imaging (e.g., using Indium-111 (111In)) provides for the ability to collect pharmacokinetic and biodistribution data that can be used to perform dosimetry calculations for treatment planning. (See, e.g., Sgouros G, Hobbs R F. “Dosimetry for radiopharmaceutical therapy.” Semin Nucl Med. 2014 May; 44(3):172-8).). Without being bound by theory, a quantitative demonstration of targeting observed with an imaging label is indicative of the ability to target with a radiolabel (e.g., an alpha emitter) capable of causing targeted cell death. Such phenomena is illustrated by FIG. 8, which illustrates that mice labeled with the imaging isotope 111In (top), exhibit accumulation of the therapeutic isotope 225Ac in tumors that express low amounts of antigen and high amounts of antigen, in this example DLL3 expressing SHP77 tumors and HER2 expressing BT474 tumors respectively.

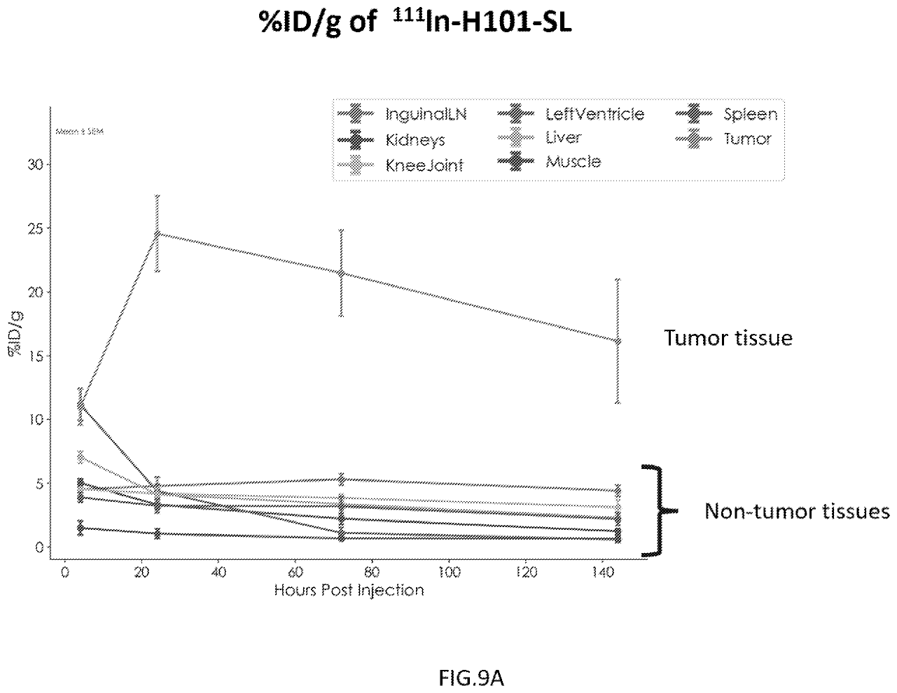

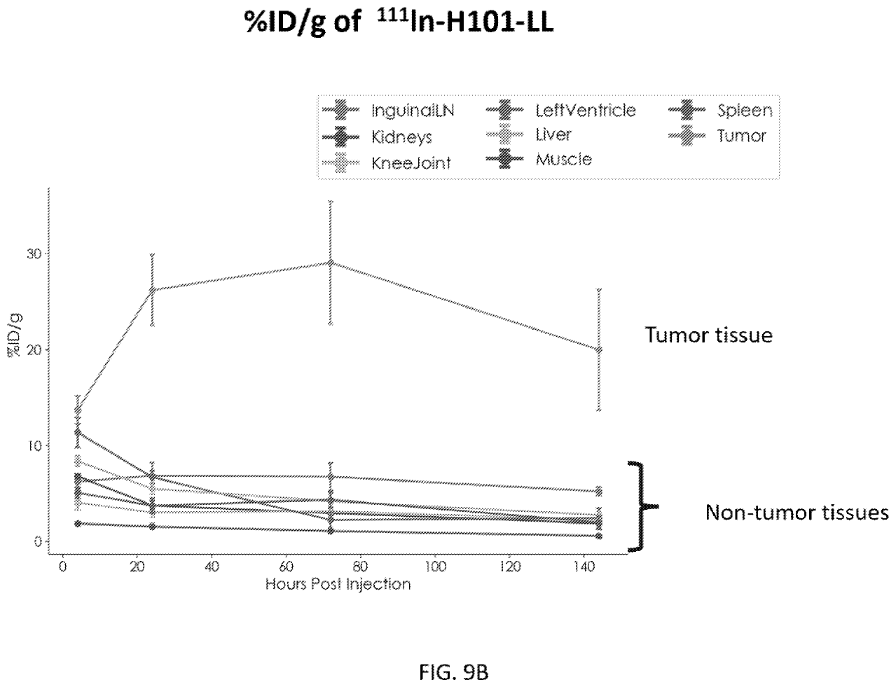

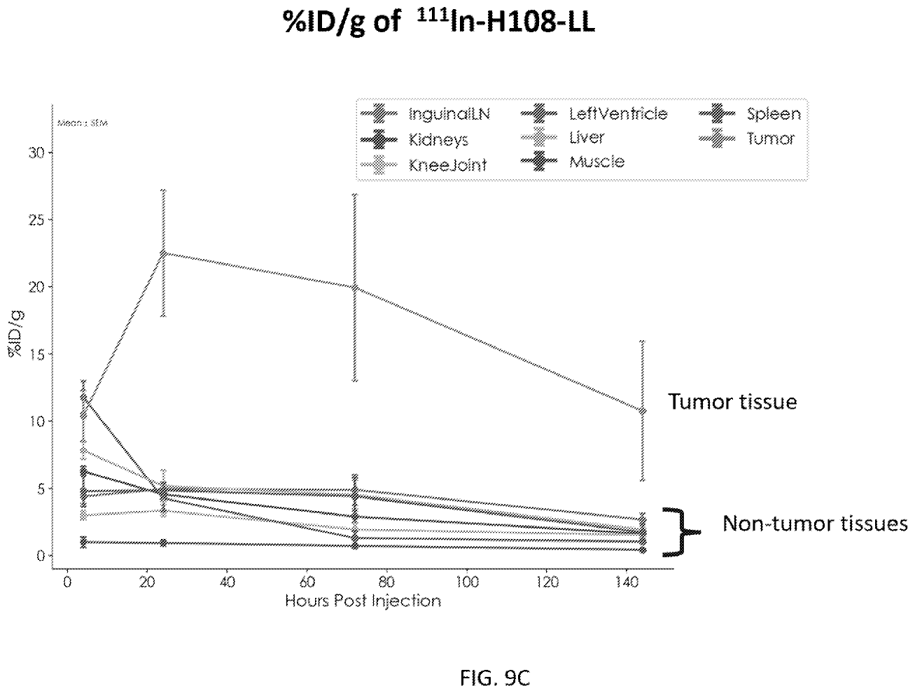

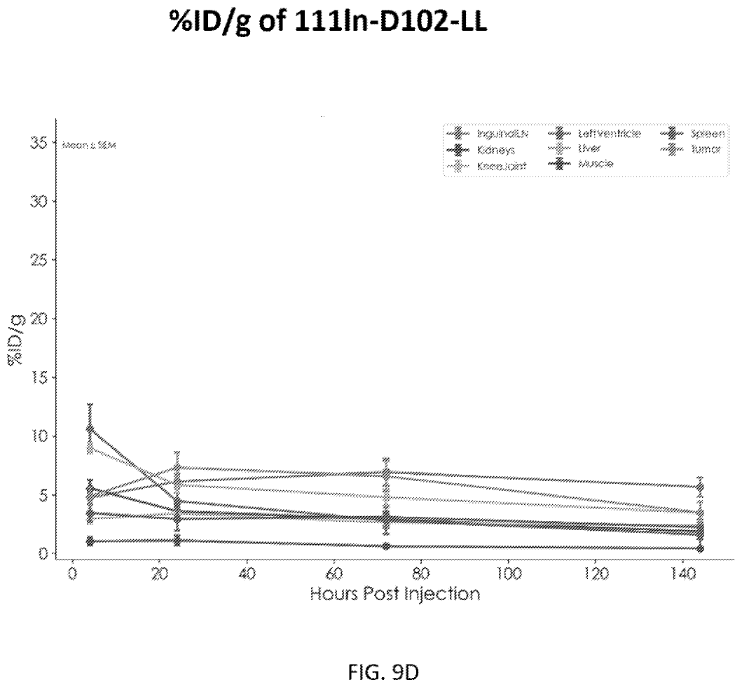

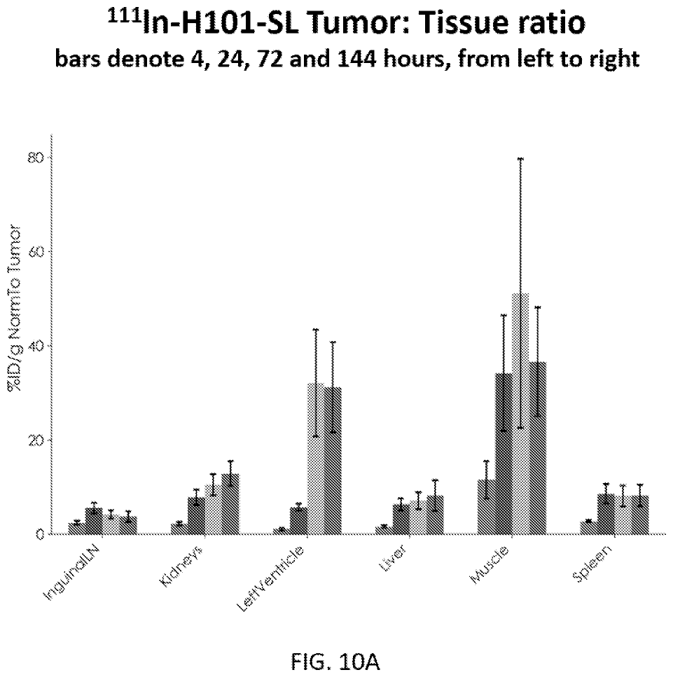

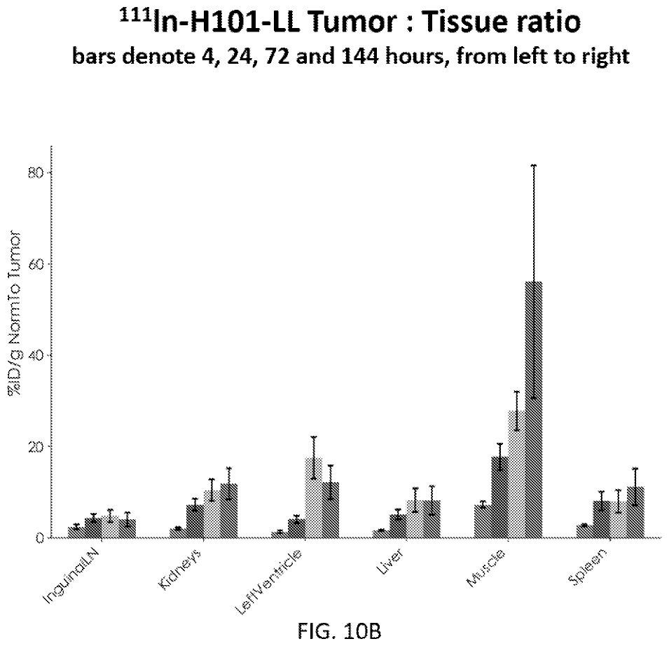

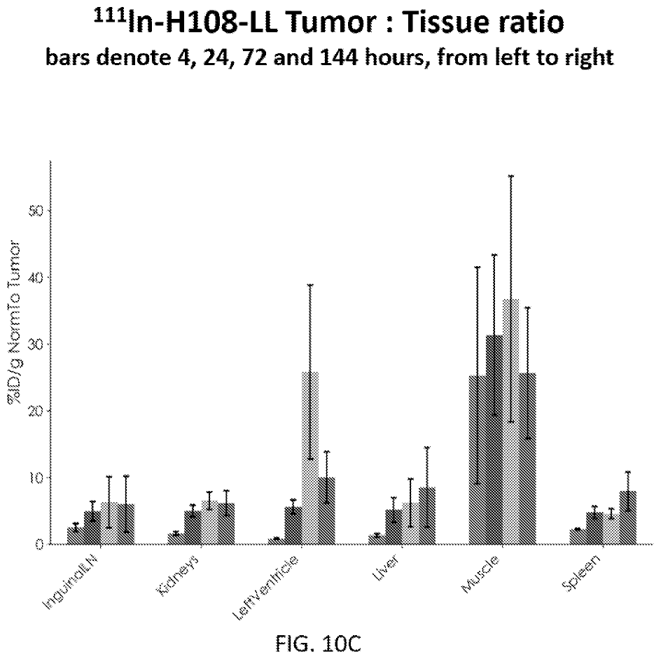

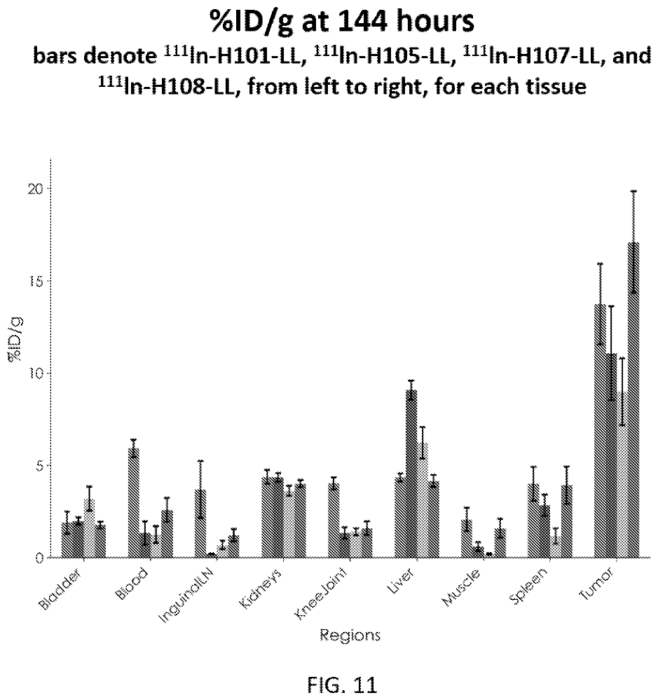

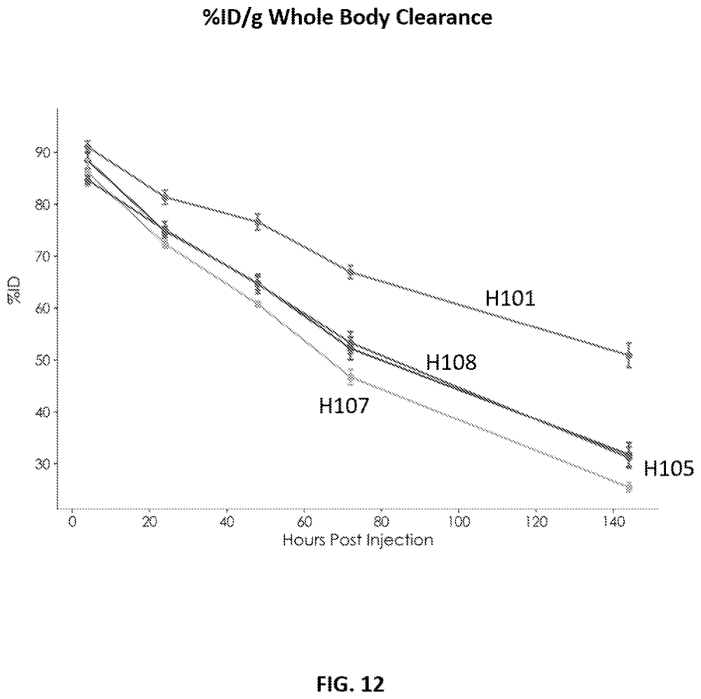

The objective of this study was to observe the biodistribution of 111In radiolabeled SPECT/CT imaging across select test articles in BT-474 tumor (breast cancer cells) bearing nude mice. The following articles were tested at a CAR of about 4: 111In-H101-short DOTA linker (p-SCN-Bn-DOTA, SL), 111In-H101-long DOTA linker (TFP-Ad-PEG5-DOTAGA, LL), 111In-H105-LL, 111In-H107-LL, and 111In-H108-LL. FIGS. 9A, 9B, and 9C show tissue accumulation over time for 111In-H101-SL, 111In-H101-LL, and 111In-H108-LL. FIG. 9D shows minimal tumor accumulation with DLL3 targeting VHH-Fc in HER2+ tumor model, further demonstrating specificity of the HER2 targeting VHH-Fcs. FIGS. 10A, 10B, and 10C show tumor:tissue ratios. In each case, the tumor:tissue ratios were greater than 5, indicating increased tumor accumulation and better profiles used for determining safety (e.g., as compared lower tumor:tissue ratios). FIG. 11 shows % ID/g at 144 hours for 111In-H101-LL, 111In-H105-LL, 111In-H107-LL, and 111In-H108-LL. In each case, the VHH-Fc variants show advantageous targeting of tumor tissue. FIG. 12 shows whole body clearance of VHH-Fc (H101) and VHH-Fc variants (H105, H107, and H108), wherein the VHH-Fc variants show increased clearance which can further be advantageous when considering safety and preventing unwanted tissue toxicity. In all cases, all test articles avoided significant kidney accumulation, further demonstrating favorable profiles for safety and avoiding unwanted tissue toxicity. Table 13 specifically shows the tumor accumulation for 111In-H101-LL, 111In-H105-LL, 111In-H107-LL, and 111In-H108-LL over time.

TABLE 13
Tumor accumulation of anti-HER2 VHH-Fc
variants (mean % ID/g; n = 4)

| | | 4 h | 24 h | 48 h | 72 h | 144 h |
|---|---|---|---|---|---|---|
| ¹¹¹In-H108- | mean | 4.7 | 12.2 | 14.4 | 12.7 | 13.7 |
| LL | SEM | 0.6 | 1.8 | 2.1 | 0.9 | 2.2 |
| ¹¹¹In-H101- | mean | 4.9 | 9.3 | 14.2 | 14.1 | 11.1 |
| LL | SEM | 0.5 | 1.1 | 2.0 | 2.6 | 2.6 |
| ¹¹¹In-H105- | mean | 4.9 | 7.1 | 9.0 | 9.4 | 9.0 |
| LL | SEM | 1.1 | 2.0 | 1.9 | 2.2 | 1.8 |
| ¹¹¹In-H107- | mean | 6.2 | 12.6 | 18.6 | 18.0 | 17.1 |
| LL | SEM | 1.1 | 1.9 | 2.3 | 2.6 | 2.8 |

Biodistribution and Tissue Accumulation Over Time in DLL3+SHP-77 Tumors

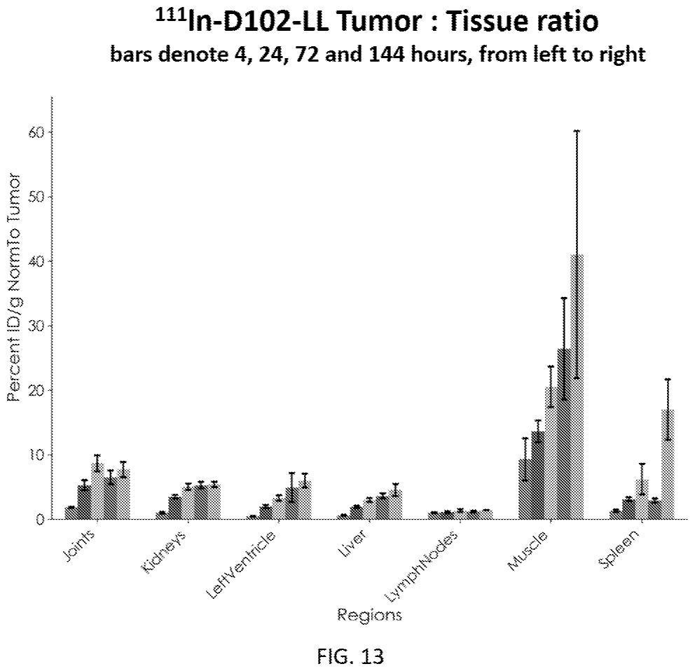

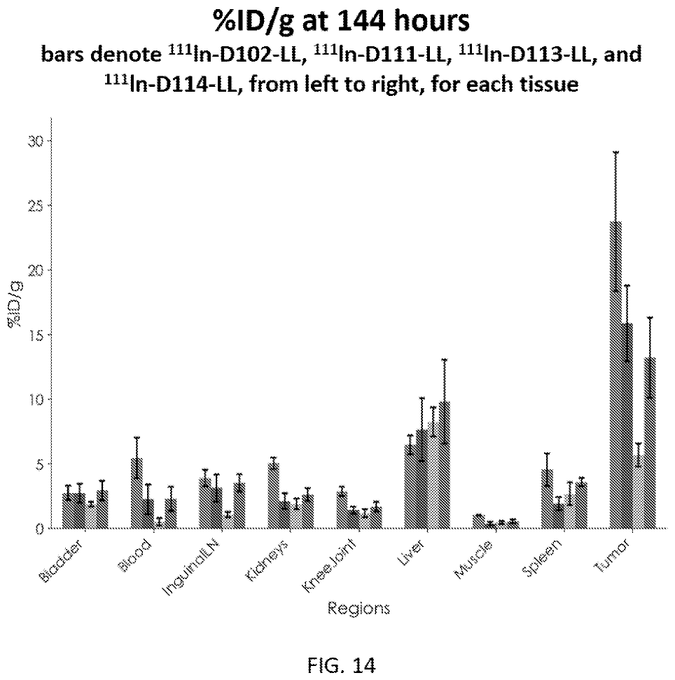

The objective of this study was to observe the biodistribution of 111In SPECT/CT across select test articles in SHP-77 tumor bearing nude mice. In contrast to HER2, DLL3 is generally present at lower copy numbers on the cell surface. Accordingly, the DLL3 represents the ability to target low copy number target proteins, whereas HER2 represents the ability to safely and effectively target high copy number target proteins. The following articles were tested: 111In-D102-long DOTA linker (LL), 111In-D111-LL, 111In-D113-LL, and 111In-D114-LL. Interestingly, similar targeting profiles and observations to the HER2 model were observed for the DLL3 model, demonstrating the ability to target high and low copy number targets. FIG. 13 shows 111In-D102-LL Tumor: Tissue ratios and FIG. 14 shows % ID/g at 144 hours for 111In-D102-LL, 111In-D111-LL, 111In-D113-LL, and 111In-D114-LL. As observed for HER2, anti-DLL3 VHH-Fc variants showed advantageous targeting of tumor tissue. Additionally, liver accumulation is indicative of increased clearance, which can further be advantageous when considering safety and preventing unwanted tissue toxicity. In all cases, all test articles avoided significant kidney accumulation, further demonstrating favorable profiles for safety and avoiding unwanted tissue toxicity. Table 14 specifically shows the tumor accumulation for 111In-D102-LL, 111In-D 111-LL, 111In-Di113-LL, and 111In-D 114-LL over time.

TABLE 14
Tumor accumulation of anti-DLL3 VHH-Fc
variants (mean % ID/g; n = 4)

| | | 4 h | 24 h | 48 h | 72 h | 144 h |
|---|---|---|---|---|---|---|
| ¹¹¹In-D102- | mean | 6.0 | 12.8 | 18.0 | 19.0 | 23.7 |
| LL | SEM | 0.7 | 1.7 | 2.1 | 2.1 | 5.4 |
| ¹¹¹In-D111- | mean | 5.5 | 12.8 | 16.6 | 16.8 | 15.9 |
| LL | SEM | 1.4 | 1.1 | 2.0 | 2.3 | 2.9 |
| ¹¹¹In-D113- | mean | 4.5 | 8.7 | 10.0 | 9.4 | 5.7 |
| LL | SEM | 0.6 | 1.2 | 1.4 | 1.2 | 0.9 |
| ¹¹¹In-D114- | mean | 5.1 | 10.9 | 14.6 | 15.8 | 13.2 |
| LL | SEM | 0.5 | 0.9 | 1.6 | 2.4 | 3.1 |

Taken together, the ¹¹¹In imaging results show that targeting of both high copy number and low copy number targets can be achieved with the radiolabeled VHH-Fcs and VHH-Fc variants. These results further indicate favorable safety and specificity profiles for targeting tumor tissue, avoiding non-tumor tissue, and in certain instances, effectively clearing radiolabeled VHH-Fcs (e.g., VHH-Fcs having mutations that reduced FcRn affinity).

### Biodistribution and Tissue Accumulation of Ac-225 Radiolabeled VHH-Fcs

The objective of this study was to observe biodistribution of (i) Ac-225 radiolabeled HER2 VHH-Fcs in a BT-474 tumor mouse model, as described above, and (ii) Ac-225 radiolabeled DLL3 VHH-Fcs in a SHP-77 tumor mouse model, as described above. Ex vivo radioactive quantitation in tumor and normal tissues was achieved by gamma counting.

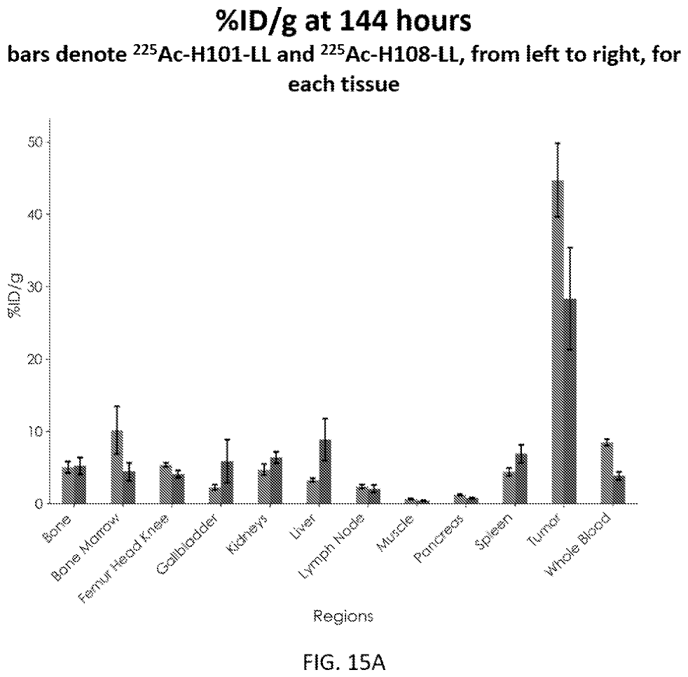

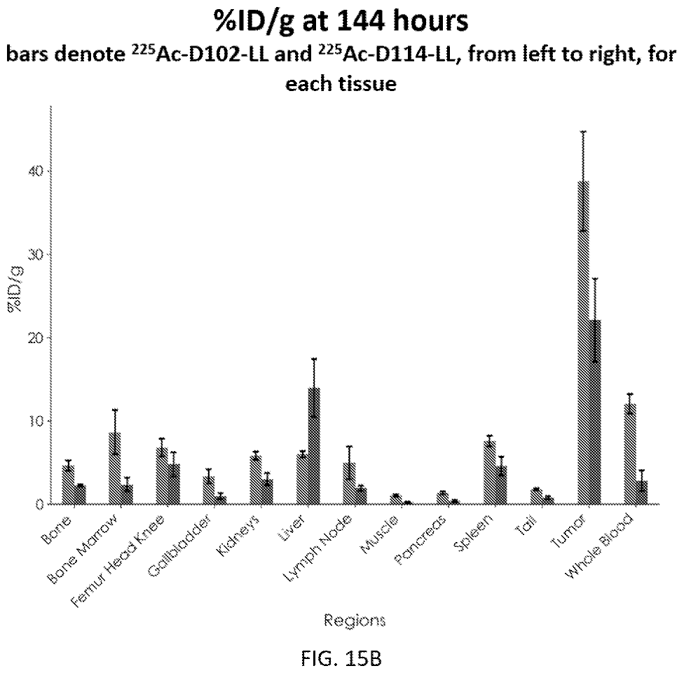

As described herein, the HER2 model represents a target with high receptor density on cancer cells (e.g., ˜300,000 copies/cell). FIG. 15A shows % ID/g at 144 hours for 225Ac-H101-LL and 225Ac-H108-LL. Both test articles showed advantageous targeting profiles, consistent with the 111In imaging data. Notably, specific targeting of tumor tissue was achieved with a favorable tumor:tissue ratio consistent with the imaging data. For the VHH-Fc variant 225Ac-H108-LL, lower radioactivity was detected in blood indicating more rapid clearance of the VHH-Fc variant (consistent with results in Example 10). 225Ac-H108-LL also demonstrated lesser kidney accumulation and greater liver accumulation indicating increased clearance through the hepatic route and avoidance of the kidneys which further supports an increase in the safety profile of VHH-Fcs with FcRn mutations. The lower tumor accumulation for 225Ac-H108-LL can be attributed to the decreased serum half-life (i.e., more rapid clearance). Table 15 further shows tumor volume through Day 6 post injection, wherein tumor volumes decreased after administration of 225Ac-H101-LL and 225Ac-H108-LL. Table 15 indicates that mice injected with VHH immunoconjugates with wild-type Fc or with FcRn mutations both saw tumor shrinkage by 6 days post injection.

TABLE 15
Tumor volumes before and after anti-HER2
VHH-Fc treatment (mean mm³; n = 5)

| | Day | −15 | −11 | −8 | −6 | −4 | −1 | 0 (dose) | 3 | 6 |
|---|---|---|---|---|---|---|---|---|---|---|
| ²²⁵AC- | mean | 57.4 | 66.5 | 51.9 | 54.7 | 65.6 | 73.9 | 74.4 | 31.3 | 47.0 |
| H101-LL | SD | 19 | 10 | 10 | 10 | 20 | 36 | 22 | 11 | 12 |
| ²²⁵AC- | mean | 46.4 | 56.5 | 67.9 | 63.2 | 67.3 | 62.6 | 78.1 | 46.2 | 51.2 |
| H108-LL | SD | 9 | 11 | 16 | 12 | 14 | 14 | 27 | 19 | 23 |

As also described herein, DLL3 represents a target with low target density on cancer cells (e.g., ˜3,000 copies/cell). FIG. 15B shows % ID/g at 144 hours for 225Ac-D102-LL and 225Ac-D114-LL. Both test articles showed advantageous targeting profiles, consistent with the 111Ln imaging data. Additionally, specific targeting of tumor tissue was achieved with a favorable tumor:tissue ratio consistent with the imaging data. As observed with the anti-HER2 VHH-Fc variants, for the VHH-Fc variant 225Ac-D114-LL, the VHH-Fc variants show increased clearance and decreased kidney exposure which can further be advantageous when considering safety and preventing unwanted tissue toxicity. The lower tumor accumulation for 225Ac-D114-LL can be attributed to the decreased serum half-life (i.e., more rapid clearance).

### Example 24. Low Toxicity Associated with VHH-Fc Radioimmunoconjugates

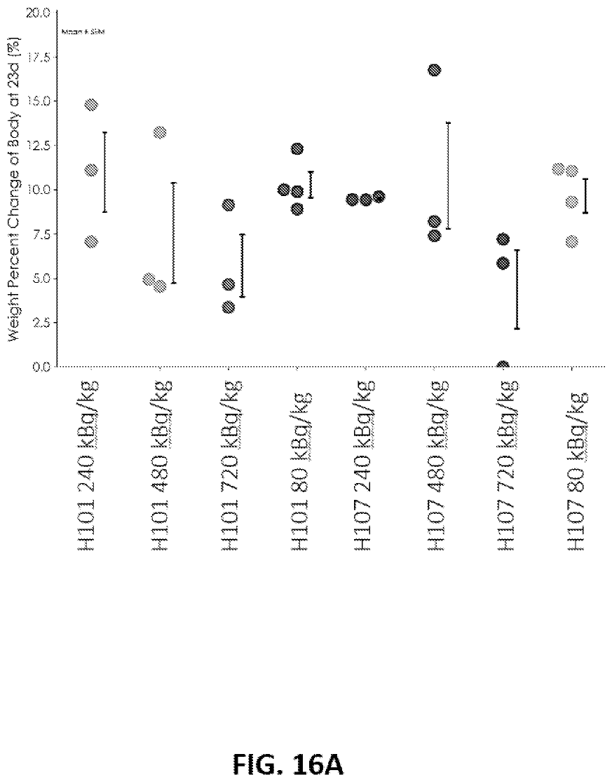

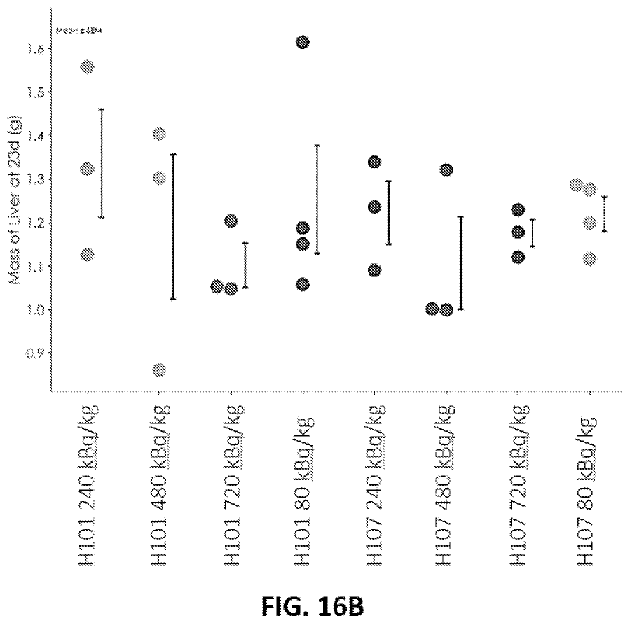

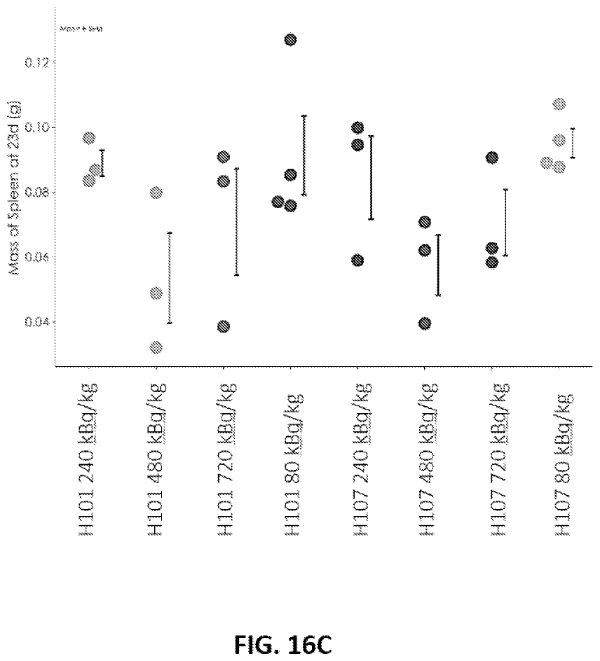

A study was undertaken to determine the tolerability of VHH-Fc loaded with 225AC. Naïve female athymic nude mice were injected intravenously (IV) into the tail vein with 225Ac-H101-447804 (anti-HER2 with wildtype Fc, TFP-Ad-PEG5-DOTAGA) or 225Ac-H107-447804 (anti-HER2 with H310A Fc, TFP-Ad-PEG5-DOTAGA) at four different activity dose levels (18.5 kBq, 12 kBq, 6 kBq, 2 kBq). Activity dose volume was adjusted for body weights measured on the injection day. All animals were monitored for adverse effects daily. Body weights were recorded three (occasionally two or four) times a week for all animals until end of study at 23 days post-injection. 23 Days post-injection all animals were sacrificed. Carcasses underwent necropsy. Whole body, spleen, and liver weights were recorded. FIGS. 16A, 16B, and 16C show that, as measured by percent weight change (16A), liver mass (16B), and spleen mass (16C) All doses of 225Ac-labeled antibodies of up to 740 kBq/kg were well tolerated and no indications of radiation sickness were observed.

### Example 25. Radiolabeling with Lu-177

50 μg of test article (D102) was diluted to 100 μL with 0.1 M ammonium acetate buffer pH 5.5 in a 500 μL lo-bind Eppendorf tube and 51 MBq in 3.2 μL-3.5 μL of 177-Lutetium chloride was added and mixed with a pipette. The reaction mixtures were incubated at 37° C. in an incubator for 3 hours and samples taken at 30 min, and 1, 2, and 3 h for iTLC analysis. Results of the labeling are shown in Table 16 below, and indicate efficient labeling with 177-Lutetium.

TABLE 16

| | |
|---|---|
| Test article | Incubation time at 37 deg C. |

| D102 | 30 min | 60 min | 2 hr | 3 hr |
|---|---|---|---|---|
| TFP-Ad- | 99.0% | 99.2% | 99.2% | 99.3% |
| PEG5- | | | | |
| DOTAGA | | | | |

After dilution in PBS/ascorbate and storage at 4° C. the purity as assessed by iTLC analysis as in Example 22.

To analyze stability, 50 μL of test article was added to 200 μL of PBS/ascorbate and stored at 4° C. The samples were analyzed by iTLC and SEC-HPLC after 1-4 h and 18-24 h. Results are shown in Table 17 below, and indicate stability of the construct.

TABLE 17

| | |
|---|---|
| Test article | Incubation time |

| D102 | 1 hr | 1 d |
|---|---|---|
| TFP-Ad-PEG5-DOTAGA | 98.8% | 98.6% |

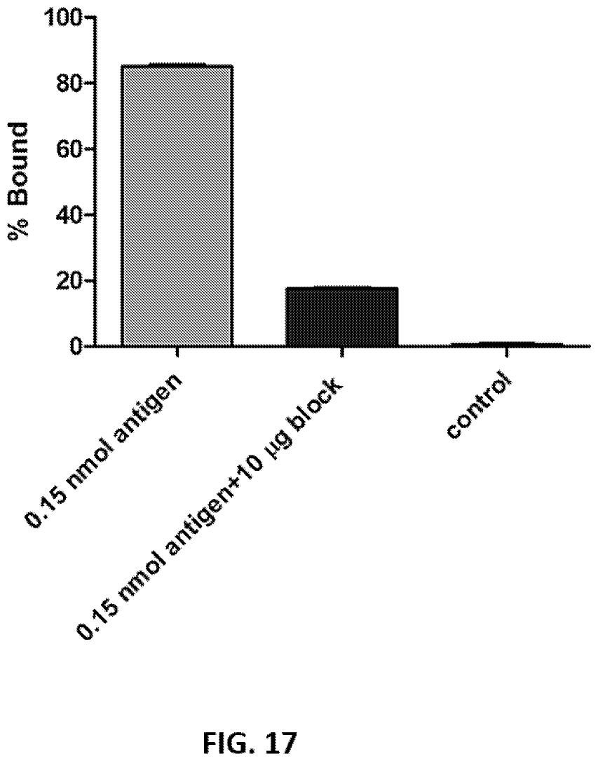

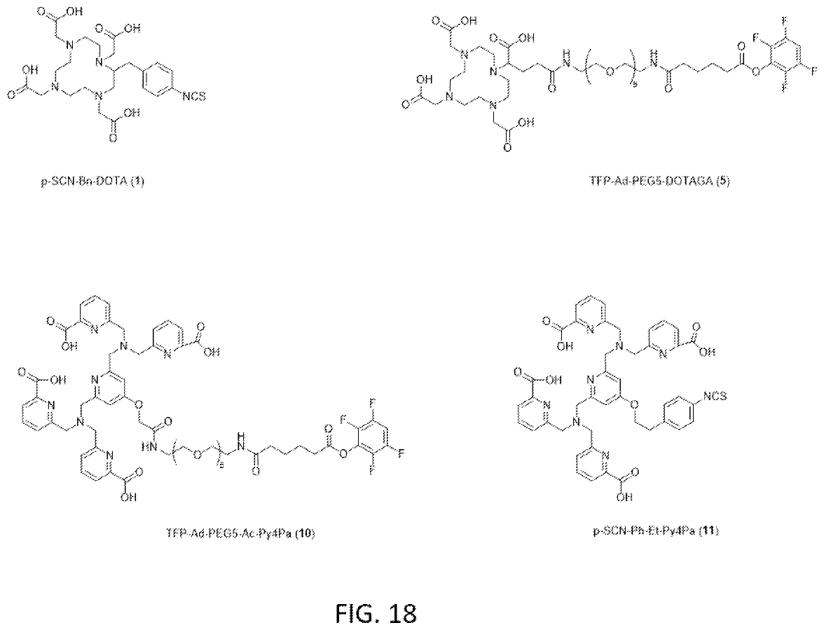

The Lu-177 conjugate was analyzed by the IRF assay described above in Example 23 and the results are shown in FIG. 17. In this example, the control is beads with no antigen loaded.

### Example 26. Multivalent Constructs

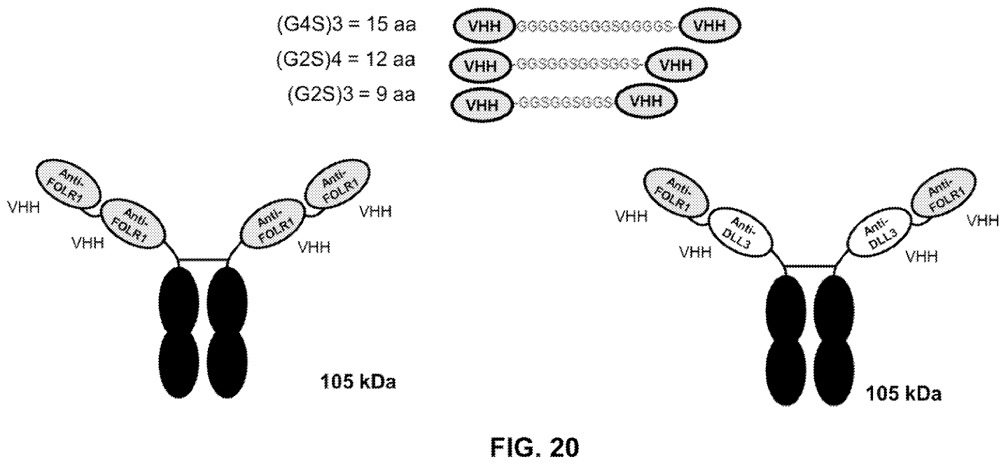

Monospecific and bispecific multivalent constructs exemplified by FIG. 19A were designed targeting FOLR1 (monospecific) or targeting both DLL3 and FOLR1 (bispecific). VHHs were coupled by a linker comprising glycine and serine amino acid residues (e.g., (GGGGS)3, (GGS)4, (GGS)3). FIG. 20 depicts the multivalent constructs described and exemplified herein. The multivalent constructs were made as described herein. Both monospecific and bispecific formats were produced with varying linker lengths. All constructs showed were able to be produced and showed 100% monomeric purification, as shown in Table 25. FOLR1 binder (SEQ ID: 4); DLL3 binder (SEQ ID NO: 8).

TABLE 18

| | | | | Mol. Weight | Yield | % |
|---|---|---|---|---|---|---|
| VHH | Fc | Target | Linker | (Da) | Mg/100 mL | Monomer |
| Anti-FOLR1x4 | Wt | FOLR1 | GGGGSx3 | 103,688 | 23 | 100 |
| Anti-FOLR1x 4 | Wt | FOLR1 | GGSx4 | 103,406 | 32 | 100 |
| Anti-FOLR1x4 | Wt | FOLR1 | GGSx3 | 103,004 | 17 | 100 |
| Anti-FOLR1x2 | Wt | FOLR1 | GGGGSx3 | 104,948 | 31 | 100 |
| Anti-DLL3x2 | | DLL3 | | | | |
| Anti-FOLR1x2 | Wt | FOLR1 | GGSx4 | 104,665 | 48 | 100 |
| Anti-DLL3x2 | | DLL3 | | | | |
| Anti-FOLR1x 2 | Wt | FOLR1 | GGSx3 | 104,263 | 31 | 100 |
| Anti-DLL3x2 | | DLL3 | | | | |

Multivalent constructs were tested for binding to antigen by a standard ELISA to FOLR1 assay results are shown in Table 19.

TABLE 19

| | hFOLR1 | mFOLR1 |
|---|---|---|
| Sample | EC50 nM | EC50 nM |
| anti-hFOLR1 mIgG MAb (Sinobiologics | 0.15 | |
| 11241-MM06) | | |
| anti-mFOLR1 RbIgG PAb (Sinobiologics | | 0.06 |
| 50573-RP02) | | |
| Farletuzumab | 0.12 | nb |
| FolR1 VHH | 0.12 | 0.08 |
| DLL3 VHH | nb | nb |
| Anti-FOLR1x4 GGGGSx3 | 0.12 | 0.08 |
| Anti-FOLR1x4 GGSx4 | 0.14 | 0.11 |
| Anti-FOLR1x4 GGSx3 | 0.11 | 0.09 |
| Anti-FOLR1x2; Anti-DLL3x2; GGGGSx3 | 0.13 | 0.08 |
| Anti-FOLR1x2; Anti-DLL3x2; GGSx4 | 0.11 | 0.07 |
| Anti-FOLR1x 2; Anti-DLL3x2; GGSx3 | 0.14 | 0.09 |

### Example 27. Antibodies and Immunoconjugates Targeting Kidney

An anti-FOLR1 VHH-Fc based on either a human specific (VHH17) or a mouse cross-reactive anti-FOLR1 VHH-Fc (VHH19) binding domain tested an in vivo study using the OV90 xenograft tumor model (ovarian tumor model). VHH17 or VHH19 was administered to mice harboring OV90 xenografts. Table 20 shows binding data for VHH17 and VHH 19.

TABLE 20

| | | | | | Human | Mouse |
|---|---|---|---|---|---|---|
| | | | | | FOLR1 | FOLR1 |
| | Human | Mouse | OVCAR3 + | BT474 − | KD | KD |
| VHH | FOLR1 | FOLR1 | FOLR1 | FOLR1 | (nM) | (nM) |
| 17 | ++ | − | ++ | − | 10.8 | − |
| 19 | ++ | ++ | ++ | − | 3.1 | 8.1 |

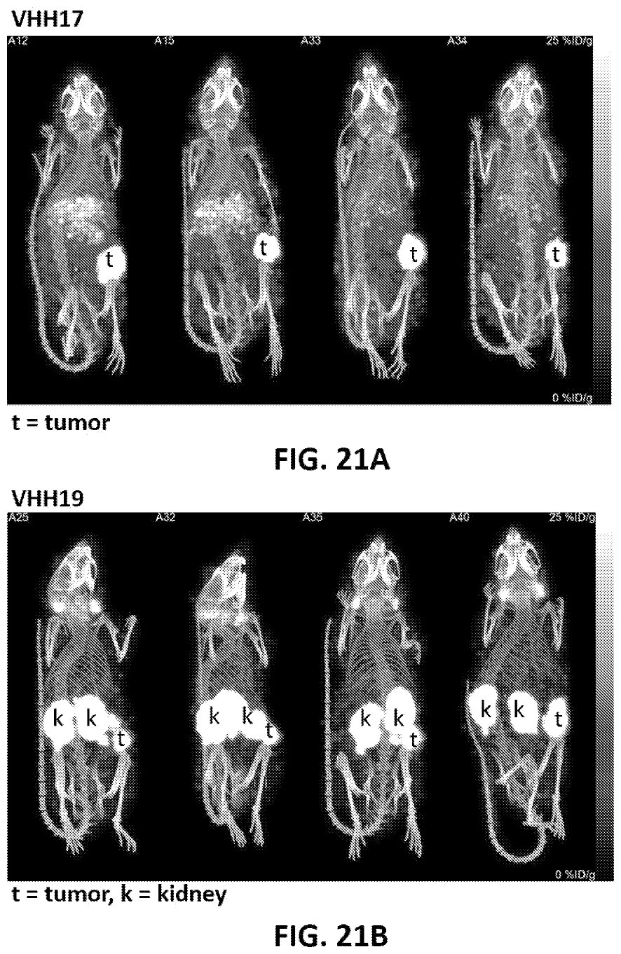

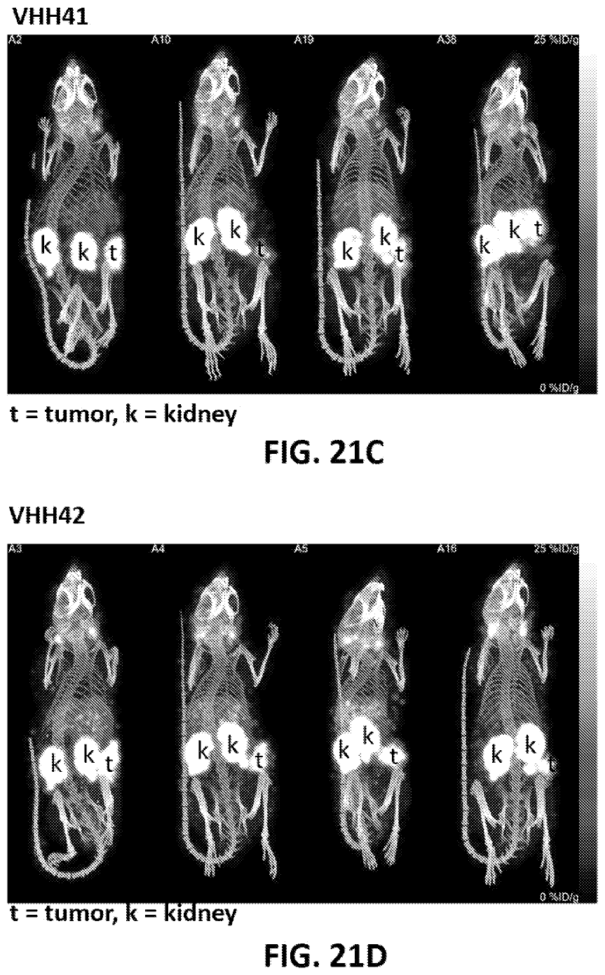

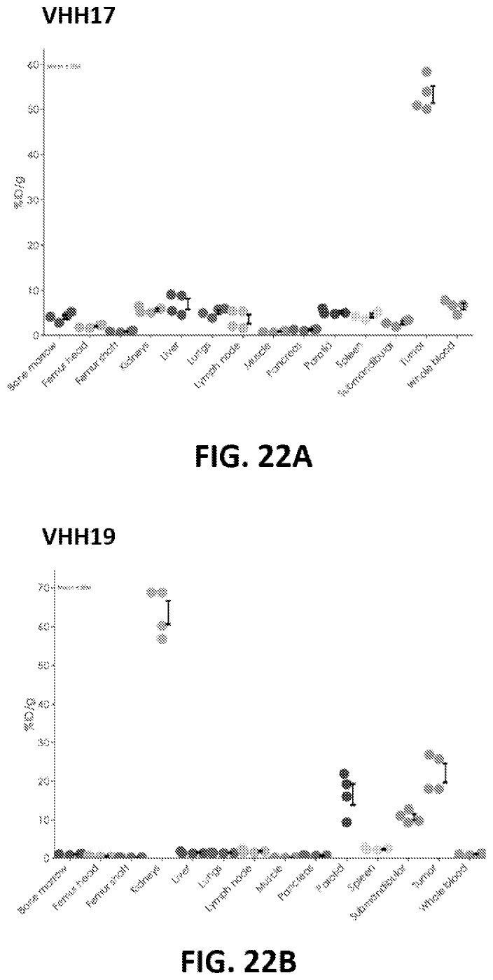

VHH-WT-Fc immunoconjugates were labeled with ¹¹¹In. Both human-specific (VHH17) and mouse cross-reactive VHH-Fc (VHH19) showed high tumor accumulation. FIGS. 21A to 21D show the tumor localization in mice 72-hours post administration. FIGS. 22A and 22B show biodistribution of human (VHH17) and mouse cross-reactive VHH-Fc (VHH19). Human-specific VHH17 showed high tumor localization/uptake (FIG. 22A). Mouse cross-reactive VHH19 (WT-Fc), VHH41 (H310A-Fc), and VHH42 (H435Q-Fc) showed effective tumor distribution but also high kidney localization/uptake, demonstrating that the VHH-Fc format can be used to target an antigen present within the kidney (FIG. 22B). Detailed image analysis of kidney distribution further indicated that localization of the kidney primarily occurred within the kidney cortex, consistent with uptake in proximal tubules. Compared to VHH19, VHH41 and VHH42 showed increased uptake in liver and faster clearance resulting from the Fc mutation.

VHH-Fc VHH19 comprising Fc mutation H310A (VHH41) or H435Q (VHH42) was also tested. FIG. 22A shows tumor and kidney localization/uptake of VHH41 and FIG. 22A shows tumor and kidney localization/uptake of VHH42 72-hours post administration.

### Example 28. Binding to FOLR1 or DLL3 Target Antigen

Tetramers and VHH-Fcs were assessed for affinity to target antigens. AHC Octet biosensors were used to capture tetramer/VHH-Fc and then dipped into antigen. All tetramers bound to human and murine FOLR1 as expected (See Table 21 and 22). The 3 bispecific tetramers were tested for binding to human and murine DLL3 and showed target binding, despite their potentially more restrictive location within the construct design, adjacent to the hinge/Fc.

TABLE 21

| | KD (nM) | Ka (1/Ms) | Kd (1/s) | X\hat{ }2 | R\hat{ }2 |
|---|---|---|---|---|---|
| | hFOLR1 | | | | |
| Anti-FOLR1x4 (GGGGS)₃ | 2.859 | 3.332E+05 | 9.526E−04 | 2.0924 | 0.9976 |
| Anti-FOLR1x4 (GGS)₄ | 2.849 | 2.993E+05 | 8.526E−04 | 2.3047 | 0.9977 |
| Anti-FOLR1x4 (GGS)₃ | 2.519 | 3.452E+05 | 8.695E−04 | 1.8678 | 0.9973 |
| Anti-FOLR1x2; Anti- | 2.747 | 4.052E+05 | 1.113E−03 | 2.6274 | 0.9961 |
| DLL3x2; (GGGGS)3 | | | | | |
| Anti-FOLR1x2; Anti- | 2.324 | 4.009E+05 | 9.318E−04 | 1.8698 | 0.993 |
| DLL3x2; (GGS)₄ | | | | | |
| Anti-FOLR1x2; Anti- | 2.678 | 3.809E+05 | 1.02E−03 | 2.7207 | 0.9947 |
| DLL3x2; (GGS)₃ | | | | | |
| FOLR1 VHH | 2.185 | 3.788E+05 | 8.278E−04 | 2.377 | 0.9958 |
| DLL3 VHH | No binding | | | | |
| | mFOLR1 | | | | |
| Anti-FOLR1x4 (GGGGS)₃ | 6.595 | 4.709E+05 | 3.105E−03 | 2.2162 | 0.9919 |
| Anti-FOLR1x4 (GGS)₄ | 5.999 | 4.351E+05 | 2.610E−03 | 2.775 | 0.99 |
| Anti-FOLR1x4 (GGS)₃ | 5.278 | 5.076E+05 | 2.679E−03 | 2.3496 | 0.9876 |
| Anti-FOLR1x2; Anti- | 8.320 | 4.895E+05 | 4.072E−03 | 1.2964 | 0.9941 |
| DLL3x2; (GGGGS)₃ | | | | | |
| Anti-FOLR1x2; Anti- | 7.988 | 4.831E+05 | 3.859E−03 | 1.7276 | 0.9911 |
| DLL3x2; (GGS)₄ | | | | | |
| Anti-FOLR1x2; Anti- | 7.000 | 5.527E+05 | 3.986E−03 | 1.9229 | 0.9889 |
| DLL3x2; (GGS)₃ | | | | | |
| FOLR1 VHH | 4.672 | 6.680E+05 | 3.121E−03 | 3.3926 | 0.9833 |

| | |
|---|---|
| DLL3 VHH | No binding |

TABLE 22

| | KD (nM) | Ka (1/Ms) | Kd (1/s) | X\hat{ }2 | R\hat{ }2 |
|---|---|---|---|---|---|
| | hDLL3 | | | | |
| Anti-FOLR1x2; Anti- | 1.53 | 2.00E+05 | 3.06E−04 | 4.0815 | 0.9992 |
| DLL3x2; (GGGGS)₃ | | | | | |
| Anti-FOLR1x2; Anti- | 1.24 | 2.15E+05 | 2.67E−04 | 4.9413 | 0.999 |
| DLL3x2; (GGS)₄ | | | | | |
| Anti-FOLR1x2; Anti- | 1.43 | 2.19E+05 | 3.12E−04 | 3.0834 | 0.9994 |
| DLL3x2; (GGS)₃ | | | | | |
| DLL3 VHH | 2.29 | 1.66E+05 | 3.79E−04 | 5.497 | 0.9992 |
| FOLR1 VHH | No binding | | | | |
| | mDLL3 | | | | |
| Anti-FOLR1x4 (GGGGS)₃ | 3.09 | 2.22E+05 | 6.84E−04 | 3.0714 | 0.999 |
| Anti-FOLR1x4 (GGS)₄ | 2.88 | 2.23E+05 | 6.42E−04 | 3.0221 | 0.999 |
| Anti-FOLR1x4 (GGS)₃ | 2.92 | 2.26E+05 | 6.59E−04 | 3.0606 | 0.999 |
| DLL3 VHH | 1.24 | 2.15E+05 | 2.67E−04 | 4.9413 | 0.999 |

| | |
|---|---|
| FOLR1 VHH | No binding |

### Example 29. Antibodies and Immunoconjugates Targeting Kidney

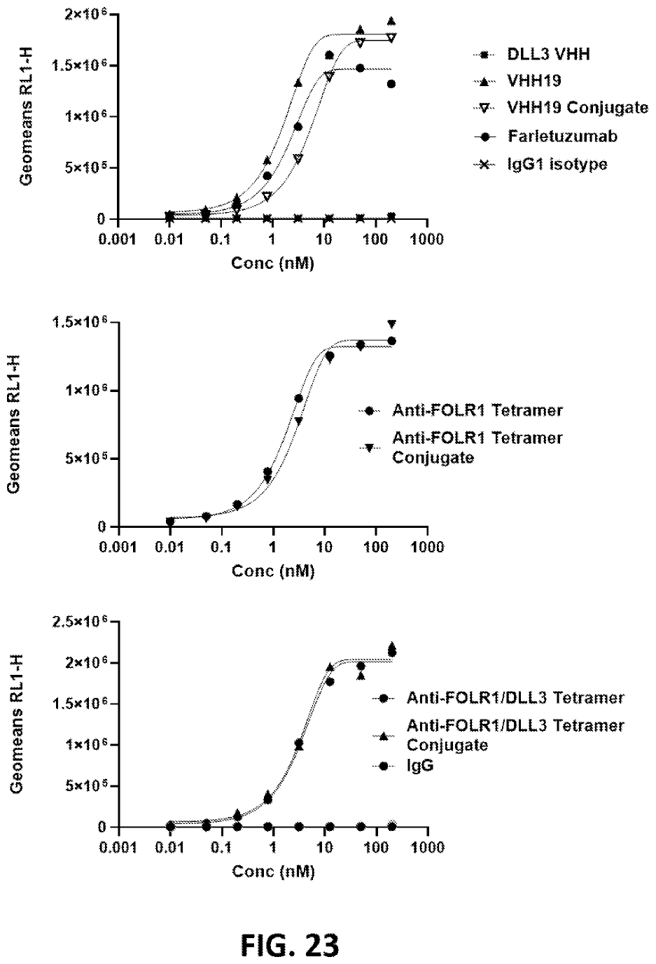

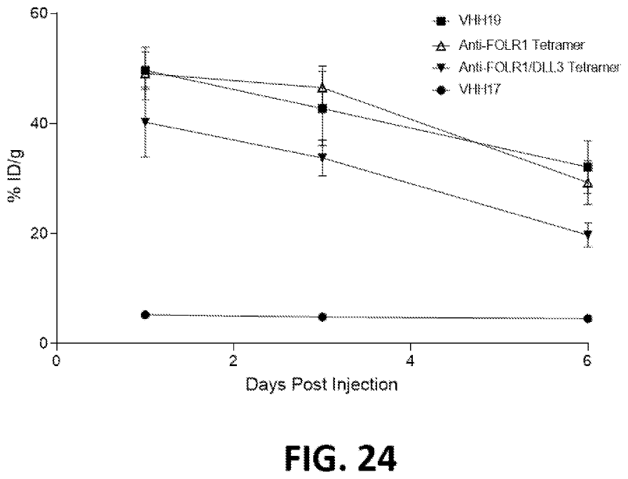

Human/mouse cross-reactive anti-FOLR1 VHHFc, VHH19, was conjugated with p-SCN-Bn-DOTA, as was the monospecific anti-h/mFOLR1 tetramer with (GGS)3 linker and the bispecific anti-h/mFOLR1/h/mDLL3 (GGS)3 tetramer. The human FOLR1 specific VHHFc, VHH17 was conjugated with TFP-Ad-PEG5-DOTA, as described above. Binding to FOLR1 expressing OV-90 cells was confirmed (see FIG. 23). In vivo kidney biodistribution of In-111 FOLR1-targeting conjugates in CD1 Mice: In-111-labeled conjugate biodistribution was evaluated at 24-, 72-, and 144-hours post-treatment in naive CD1 mice. Mice (n=3-4/group) were imaged using SPECT/CT and an imaging dose of In-111 tracer (10 MBq, 1 mg/kg, 0.5 MBq/g specific activity). Kidney uptake and retention are seen with all mouse-cross reactive conjugates, as shown in FIG. 24.

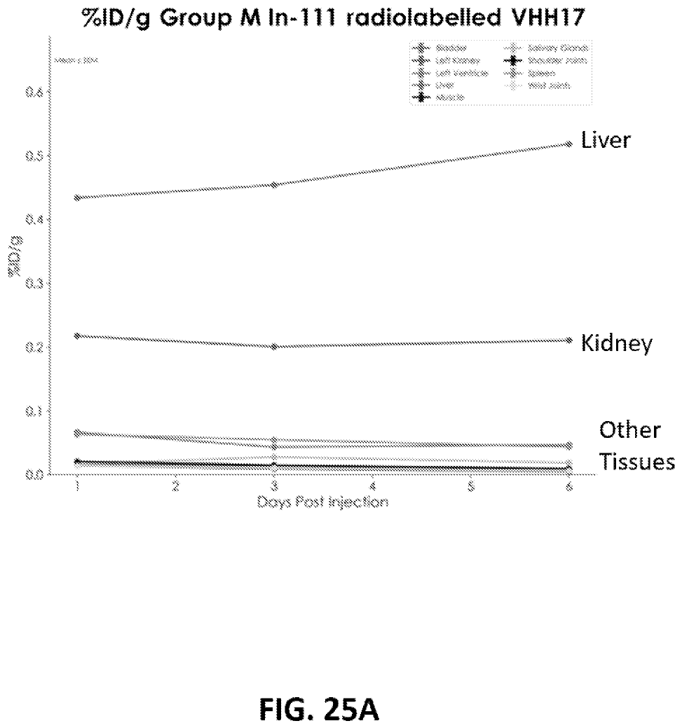

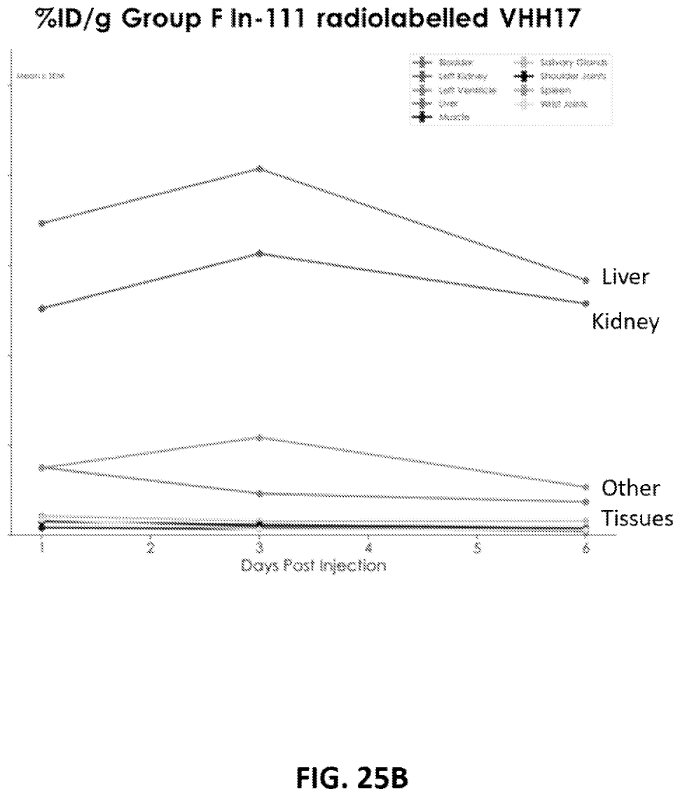

Biodistribution of In-111 radiolabeled VHH17 in cynomolgus monkeys: The human FOLR1 specific VHH-Fc VHH17 was conjugated with p-SCN-Bn-DOTA and In-111 radiolabeled. Naïve cynomolgous monkeys (n=1/sex) were dosed with test article (130 MBq, 0.3 mg/kg, 0.1 MBq/ug specific activity) and imaged using SPECT/CT at 24 h, 72 h and 144 h post-injection. In-111 radioactivity within each region of interest was quantified across all time points as percent of injected dose per gram (% ID/g). VHH17 shows high kidney biodistribution (FIGS. 25A-B). Terminal half-life was 2.3 days(M) and 3.2 days(F) using left ventricle % ID/g as a surrogate for blood pK.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

SEQUENCES

| SEQ_ID | SEQUENCE | ANNOTATION |
|---|---|---|
| 1 | GFTVSNSVMA | FOLR1_CDR-1 |
| 2 | IINSIGITNYADSVKG | FOLR1_CDR-2 |
| 3 | NFDRIY | FOLR1_CDR-3 |
| 4 | QVQLVESGGGLVQPGGSLRLSCAASGFTVSNSVMAWYRQTPGNE | FOLR1_VAR |
| | REFVAIINSIGITNYADSVKGRFTISRDNSKNTLYLQMNSLRAE | |
| | DTAVYVCNRNFDRIYWGQGTLVTVSS | |
| 5 | GSIFSINA | DLL3_CDR-1 |
| 6 | FTGDTNT | DLL3_CDR-2 |
| 7 | AADVQLFSRDYEFY | DLL3_CDR-3 |
| 8 | EVQLVESGGGEVQPGGSLRLSCAASGSIFSINAMGWYRQAPGKQ | DLL3_VAR |
| | RELVAGFTGDTNTIYAESVKGRFTISRDNAKNTVYLQMSSLRAE | |
| | DTAVYYCAADVQLFSRDYEFYWGQGTLVTVKP | |
| 9 | QVQLVESGGGLVQPGGSLRLSCEASGTTFSRDVMGWYRQAPGKQ | VHH17 |
| | RELVAIISRGGSTNYADSVKGRFTISRDNSKNTLYLQMNSLRAE | |
| | DTAVYYCNANTATWGRVFWGQGTLVTVSS | |
| 10 | QVQLVESGGGLVQPGGSLRLSCAASGFTVSNSVMAWYRQTPGNE | VHH19 |
| | REFVAIINSIGITNYADSVKGRFTISRDNSKNTLYLQMNSLRAE | |
| | DTAVYVCNRNFDRIYWGQGTLVTVSS | |
| 11 | APELLGGPSVFLFPPKPKDTLMASRTPEVTCVVVDVSHEDPEVK | FC1_I253A |
| | FNWYVDGVEVHNAKTKPREEQYNSTYRVVSVLTVLHQDWLNGKE | |
| | YKCKVSNKALPAPIEKTISKAKGQPREPQVYTLPPSREEMTKNQ | |
| | VSLTCLVKGFYPSDIAVEWESNGQPENNYKTTPPVLDSDGSFFL | |
| | YSKLTVDKSRWQQGNVFSCSVMHEALHNHYTQKSLSLSPG | |
| 12 | APELLGGPSVFLFPPKPKDTLMIARTPEVTCVVVDVSHEDPEVK | FC2_S254A |
| | FNWYVDGVEVHNAKTKPREEQYNSTYRVVSVLTVLHQDWLNGKE | |
| | YKCKVSNKALPAPIEKTISKAKGQPREPQVYTLPPSREEMTKNQ | |
| | VSLTCLVKGFYPSDIAVEWESNGQPENNYKTTPPVLDSDGSFFL | |
| | YSKLTVDKSRWQQGNVFSCSVMHEALHNHYTQKSLSLSPG | |
| 13 | APELLGGPSVFLFPPKPKDTLMISRTPEVTCVVVDVSHEDPEVK | FC3_H310A |
| | FNWYVDGVEVHNAKTKPREEQYNSTYRVVSVLTVLAQDWLNGKE | |
| | YKCKVSNKALPAPIEKTISKAKGQPREPQVYTLPPSREEMTKNQ | |
| | VSLTCLVKGFYPSDIAVEWESNGQPENNYKTTPPVLDSDGSFFL | |
| | YSKLTVDKSRWQQGNVFSCSVMHEALHNHYTQKSLSLSPG | |
| 14 | APELLGGPSVFLFPPKPKDTLMISRTPEVTCVVVDVSHEDPEVK | FC4_H435Q |
| | FNWYVDGVEVHNAKTKPREEQYNSTYRVVSVLTVLHQDWLNGKE | |
| | YKCKVSNKALPAPIEKTISKAKGQPREPQVYTLPPSREEMTKNQ | |
| | VSLTCLVKGFYPSDIAVEWESNGQPENNYKTTPPVLDSDGSFFL | |
| | YSKLTVDKSRWQQGNVFSCSVMHEALHNQYTQKSLSLSPG | |
| 15 | APELLGGPSVFLFPPKPKDTLMISRTPEVTCVVVDVSHEDPEVK | FC5_Y436A |
| | FNWYVDGVEVHNAKTKPREEQYNSTYRVVSVLTVLHQDWLNGKE | |
| | YKCKVSNKALPAPIEKTISKAKGQPREPQVYTLPPSREEMTKNQ | |
| | VSLTCLVKGFYPSDIAVEWESNGQPENNYKTTPPVLDSDGSFFL | |
| | YSKLTVDKSRWQQGNVFSCSVMHEALHNHATQKSLSLSPG | |
| 16 | APELLGGPSVFLFPPKPKDTLMISRTPEVTCVVVDVSHEDPEVK | FC6_HAHQ |
| | FNWYVDGVEVHNAKTKPREEQYNSTYRVVSVLTVLAQDWLNGKE | |
| | YKCKVSNKALPAPIEKTISKAKGQPREPQVYTLPPSREEMTKNQ | |
| | VSLTCLVKGFYPSDIAVEWESNGQPENNYKTTPPVLDSDGSFFL | |
| | YSKLTVDKSRWQQGNVFSCSVMHEALHNQYTQKSLSLSPG | |
| 17 | APEAEGAPSVFLFPPKPKDTLMISRTPEVTCVVVDVSHEDPEVK | FC7_AEASS |
| | FNWYVDGVEVHNAKTKPREEQYNSTYRVVSVLTVLHQDWLNGKE | |
| | YKCKVSNKALPSSIEKTISKAKGQPREPQVYTLPPSREEMTKNQ | |
| | VSLTCLVKGFYPSDIAVEWESNGQPENNYKTTPPVLDSDGSFFL | |
| | YSKLTVDKSRWQQGNVFSCSVMHEALHNHYTQKSLSLSPG | |
| 18 | APEAEGAPSVFLFPPKPKDTLMISRTPEVTCVVVDVSHEDPEVK | FC8_AEASS- |
| | FNWYVDGVEVHNAKTKPREEQYNSTYRVVSVLTVLAQDWLNGKE | H310A |
| | YKCKVSNKALPSSIEKTISKAKGQPREPQVYTLPPSREEMTKNQ | |
| | VSLTCLVKGFYPSDIAVEWESNGQPENNYKTTPPVLDSDGSFFL | |
| | YSKLTVDKSRWQQGNVFSCSVMHEALHNHYTQKSLSLSPG | |
| 19 | APEAEGAPSVFLFPPKPKDTLMISRTPEVTCVVVDVSHEDPEVK | FC9_AEASS- |
| | FNWYVDGVEVHNAKTKPREEQYNSTYRVVSVLTVLHQDWLNGKE | H435Q |
| | YKCKVSNKALPSSIEKTISKAKGQPREPQVYTLPPSREEMTKNQ | |
| | VSLTCLVKGFYPSDIAVEWESNGQPENNYKTTPPVLDSDGSFFL | |
| | YSKLTVDKSRWQQGNVFSCSVMHEALHNQYTQKSLSLSPG | |
| 20 | APELLGGPSVFLFPPKPKDTLMISRTPEVTCVVVDVSHEDPEVK | FC10_WT |
| | FNWYVDGVEVHNAKTKPREEQYNSTYRVVSVLTVLHQDWLNGKE | |
| | YKCKVSNKALPAPIEKTISKAKGQPREPQVYTLPPSREEMTKNQ | |
| | VSLTCLVKGFYPSDIAVEWESNGQPENNYKTTPPVLDSDGSFFL | |
| | YSKLTVDKSRWQQGNVFSCSVMHEALHNHYTQKSLSLSPG | |
| 21 | GYIFNSCG | HER-2_CDR-1 |
| 22 | ISGDGDT | HER-2_CDR-2 |
| 23 | AVCYNLETY | HER-2_CDR-3 |
| 24 | QVQLQESGGGSVQAGGSLKLTCAASGYIFNSCGMGWYRQSPGRE | HER-2_VAR |
| | RELVSRISGDGDTWHKESVKGRFTISQDNVKKTLYLQMNSLKPE | |
| | DTAVYFCAVCYNLETYWGQGTQVTVSS | |

1. An antibody comprising:
an antigen-binding domain and a Fc domain, wherein the antigen-binding domain binds an antigen present within the kidney, wherein the antibody possesses a molecular weight of less than 110,000 Daltons.

2. The antibody of claim 1, wherein the antigen-binding domain comprises an immunoglobulin single-chain variable domain polypeptide.

3. The antibody of claim 2, wherein the immunoglobulin single-chain variable domain polypeptide comprises a VHH.

4. The antibody of any one of claims 1 to 3, wherein the Fc domain comprises a CH2 domain and CH3 domain.

5. The antibody of any one of claims 1 to 4, wherein the Fc domain comprises a CH3 domain.

6. The antibody of any one of claims 1 to 4, wherein the Fc domain comprises a CH2 domain.

7. The antibody of any one of claims 1 to 6, wherein the Fc domain comprises an alteration to one or more amino acid residues that reduces an effector function of the Fc domain.

8. The antibody of claim 7, wherein the alteration to one or more amino acid residues that reduces the effector function of the immunoglobulin heavy chain constant region is an alteration that reduces complement dependent cytotoxicity (CDC), antibody-dependent cell-cytotoxicity (ADCC), antibody-dependent cell-phagocytosis ADCP, or a combination thereof.

9. The antibody of any one of claims 1 to 8, wherein the alteration to one or more amino acid residues that reduces the effector function of the immunoglobulin heavy chain constant region is selected from the list consisting of: (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) K322A, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) E233P, (ooo) L234A, L235E, G237A, A330S, and P331S or (ppp) any combination of (a)-(ppp), per EU numbering.

10. The antibody of any one of claims 1 to 9, wherein the alteration to one or more amino acid residues that reduces the effector function of the immunoglobulin heavy chain constant region comprises L234A, L235E, G237A, A330S, and P331S per EU numbering.

11. The antibody of any one of claims 1 to 10, wherein the Fc domain comprises an alteration to one or more amino acid residues that alters binding of the antibody to the neonatal Fc receptor (FcRn).

12. The antibody of any one of claims 1 to 11, wherein the alteration to one or more amino acid residues that alters binding of the antibody to the neonatal Fc receptor (FcRn) is to an amino acid residue selected from the list consisting of: I253A, I253D, I253P, S254A, H310A, H310D, H310E, H310Q, H435A, H435Q, Y436A, and combinations thereof per EU numbering.

13. The antibody of any one of claims 1 to 11, wherein the alteration to one or more amino acid residues that alters binding of the antibody to the neonatal Fc receptor (FcRn) is to an amino acid residue selected from the list consisting of: I253A, S254A, H310A, H435Q, Y436A and combinations thereof per EU numbering.

14. The antibody of any one of claims 1 to 11, wherein the alteration to one or more amino acid residues that alters binding of the antibody to the neonatal Fc receptor (FcRn) is to an amino acid residue selected from the list consisting of: I253A, H310A, H435Q, and combinations thereof per EU numbering.

15. The antibody of any one of claims 1 to 11, wherein the alteration to one or more amino acid residues that alters binding of the antibody to the neonatal Fc receptor (FcRn) comprises I253A per EU numbering.

16. The antibody of any one of claims 1 to 11, wherein the alteration to one or more amino acid residues that alters binding of the antibody to the neonatal Fc receptor (FcRn) comprises H310A per EU numbering.

17. The antibody of any one of claims 1 to 11, wherein the alteration to one or more amino acid residues that alters binding of the antibody to the neonatal Fc receptor (FcRn) comprises H435Q per EU numbering.

18. The antibody of any one of claims 1 to 17, wherein the antibody comprises a second antigen-binding domain and the antibody is a multivalent antibody

19. The antibody of any one of claims 1 to 18, wherein the antibody comprises a molecular weight less than about 100,000 Daltons.

20. The antibody of any one of claims 1 to 18, wherein the antibody comprises a molecular weight less than about 90,000 Daltons.

21. The antibody of any one of claims 1 to 18, wherein the antibody comprises a molecular weight less than about 80,000 Daltons.

22. The antibody of any one of claims 1 to 18, wherein the antibody comprises a molecular weight between about 60,000 Daltons and 110,000 Daltons.

23. The antibody of any one of claims 1 to 22, wherein the antigen comprises FOLR1.

24. The antibody of any one of claims 1 to 23, wherein the antigen-binding domain binds FOLR1.

25. The antibody of any one of claims 1 to 24, wherein the antigen-binding domain comprises:
a complementarity determining region (CDR) 1 comprising the amino acid sequence as set forth in SEQ ID NO: 1; a complementarity determining region (CDR) 2 comprising the amino acid sequence as set forth in SEQ ID NO: 2; and a complementarity determining region (CDR) 3 comprising the amino acid sequence as set forth in SEQ ID NO: 3.

26. The antibody of any one of claims 1 to 25, wherein the first antigen-binding domain or the second antigen-binding domain comprise an amino acid sequence at least about 85% identical to that set forth in SEQ ID NO: 4.

27. The antibody of any one of claims 1 to 26, wherein the antibody is a multivalent antibody comprising the formula: 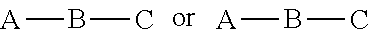
wherein:
A comprises a first antigen-binding domain;
B comprises a second antigen-binding domain; and
C comprises an Fc domain.

28. The antibody of claim 27, wherein the first antigen-binding domain binds FOLR1, second antigen-binding domain bind FOLR1, or both the first antigen-binding domain and the second antigen-binding domain each bind FOLR1.

29. A method of delivering an antibody to a kidney of an individual, the method comprising administering the antibody of any one of claims 1 to 28 to the individual.

30. A method of treating a kidney disease or disorder, the method comprising administering the antibody of any one of claims 1 to 28, to the individual.

31. The method of any one of claims 29 or 30, wherein cells within the kidney express FOLR1.

32. The method of any one of claims 29 or 30, wherein the individual is a human individual.

33. The method of claim 51, wherein the kidney disease or disorder is BK virus nephritis or nephropathy.
